(19) United States
(12) Patent Application Publication
(10) Pub. No.: US 2026/0043054 A1
(43) Pub. Date: Feb. 12, 2026

(54) GENETICALLY MODIFIED CELLS OF METHYLOBACTERIACEAE FOR FERMENTATIVE PRODUCTION OF GLYCOLIC ACID AND LACTIC ACID FROM CX COMPOUNDS

(71) Applicant: Fraunhofer-Gesellschaft zur Förderung der angewandten Forschung e.V., München (DE)

(72) Inventors: Jonathan Thomas Fabarius, Straubing (DE); Carina Sagstetter, Straubing (DE); Melanie Speck, Straubing (DE); Arne Roth, Straubing (DE)

(21) Appl. No.: 19/099,698

(22) PCT Filed: Aug. 2, 2023
(86) PCT No.: PCT/EP2023/071399

§ 371 Date: Jan. 29, 2025

(30) Foreign Application Priority Data
Aug. 3, 2022 (DE) 10 2022 119 514.7

(51) Int. Cl. C12P 7/42; C12N 9/04; C12N 9/90; C12N 15/74; C12P 7/56

(52) U.S. Cl. CPC C12P 7/42; C12N 9/0006; C12N 9/90; C12N 15/74; C12P 7/56; C12Y 101/01026; C12N 2800/101

(57) ABSTRACT

The present invention relates to a genetically modified Methylobacteriaceae cell comprising at least one exogenous nucleic acid sequence encoding a glyoxylate reductase from the bacterium Escherichia, to a process for producing the genetically modified Methylobacteriaceae cell, to a biocatalyst comprising the genetically modified Methylobacteriaceae cell, to a bioreactor comprising the genetically modified Methylobacteriaceae cell, to a process for producing a product containing glycolic acid and lactic acid, and to a process for producing polyglycolic acid, polylactic acid or polylactide-co-glycolide.

The present invention relates to a genetically modified cell from the family of Methylobacteriaceae comprising at least one exogenous nucleic acid sequence encoding a glyoxylate reductase from the bacterium Escherichia, to a process for producing the genetically modified Methylobacteriaceae cell, to a biocatalyst comprising the genetically modified Methylobacteriaceae cell, to a bioreactor comprising the biocatalyst comprising the genetically modified Methylobacteriaceae cell, to a process for producing a product containing glycolic acid and to a process for producing polyglycolic acid, polylactic acid or polylactide-co-glycolide.

Glycolic acid, also known as hydroxyacetic acid or hydroxyethanoic acid, is an organic carboxylic acid with two carbon atoms that contains a carboxyl group as a functional group and a hydroxyl group on the C₂ atom. Glycolic acid is used in a variety of ways in the textile industry, for example as a dyeing and tanning agent, in the food industry, for example as a flavouring agent and preservative agent or packaging material, and in the pharmaceutical industry, for example as a skin care product (Salusjärvi, L. et al., Applied Microbiology and Biotechnology, 2019, 103(6): p. 2525-2535; hereafter Salusjäryi et al.). In the polymer industry, glycolic acid can be processed together with lactic acid to form a co-polymer (polylactide-co-glycolide) or, in medical technology, as polyglycolic acid to form a suture material that is absorbable by the body (Salusjäryi et al.; Jem, K. J. and B. Tan, Advanced Industrial and Engineering Polymer Research, 2020. 3(2): p. 60-70; hereinafter Jem et al.). Currently, glycolic acid is industrially almost exclusively produced petrochemically from fossil raw materials via formaldehyde, carbon monoxide and water. Intensive research is being conducted into sustainable ways of producing the economically relevant compound glycolic acid from renewable raw materials, independently of fossil resources.

The production of glycolic acid from renewable substrates such as D-glucose, D-xylose, D-arabinose, L-lyxose, L-arabinose, acetates or ethanol via microbial fermentation is known, but has not yet been established industrially (Salusjäryi et al., Jem et al., Gädda, T. M. et al., Appita Journal, 2014. 67(1): p. 12). These substrates are recovered from biogenic raw materials. Therefore, there is a sustainability risk associated with the use of such substrates, since raw materials are used for chemical production that could also be used for the production of food and feed, such as bioethanol. The synthesis of glycolic acid is readily biotechnologically accessible from substrates such as hexoses, pentoses or for example glycol nitrile, as disclosed in U.S. Pat. No. 7,198,927 B2, formaldehyde and hydrogen cyanide, as disclosed in EP 1 828 393 B1, or ethylene glycol, as disclosed in EP 2 025 760 B1. In contrast thereto, the direct biotechnological synthesis of glycolic acid from CO₂ is biotechnologically difficult to access. This is inter alia due to the inherent limitations of the efficiency of photosynthetic metabolism or rather the gas-liquid mass transfer of gas fermentation. The latter two approaches are further limited by low yields and conversion rates and the number of available and genetically accessible microorganisms (Frazão, C. J. R. and T. Walther, Chemie Ingenieur Technik, 2020. 92(11): p. 1680-1699, and Kang, N. K., M. Kim, K. Baek, Y. K. Chang, D. R. Ort, and Y.-S. Jin, Chemical Engineering Journal, 2022. 433: p. 133636).

Biotechnologically useful intermediates, such as methanol or formic acid, can be produced from CO₂ in a variety of ways (Bohlen, et al., Electrochemistry Communications, 2020. 110: p. 106597; Bowker, M., ChemCatChem, 2019, 11(17): p. 4238-4246; Lénárd-Istvan Csepei, F. S. et al., F.-G.z.F.d.a.F.e.V., Editor, 2016: Germany) and will be referred to as Cx compounds in the following.

Cx compounds, for example methanol or formic acid or mixtures of these two substrates, can be used by methylotrophic microorganisms as an energy source to build biomass or valuable products, in particular chemical products. In the central carbon metabolism of methylotrophic microorganisms, Cx compounds such as methanol or formic acid are received as a substrate. In first reaction steps, methanol, for example, is oxidised to formic acid. This produces the redox equivalents required in the metabolism cytochrome C in its reduced form and NAD(P)H. Formic acid can afterwards either be oxidised to form CO₂ or (also like formaldehyde) channeled into the serine cycle. The serine cycle serves the methylotrophic microorganism as a distribution circuit of carbon and provides the main precursors required for biomass synthesis. In addition, the serine cycle is a point of attachment for further metabolic pathways that are essentially required for growth on Cx compounds. For example, in the serine cycle the intermediate glyoxylate is regenerated by the linked ethylmalonyl-CoA metabolic pathway. The serine cycle intermediate glyoxylate can be converted into glycolic acid by reduction with NADH or NADPH coupled to a glyoxylate reductase (ghrA). Glyoxylate reductases (ghrA) and hydroxypyruvate reductases (ghrB) belong to the family of glyoxylate/hydroxypyruvate reductases (ghr).

It is known that a DNA sequence is present in the M. extorquens TK 0001 genome, which encodes an endogenous glyoxylate reductase (EC: 1.1.1.26, www.ncbi.nlm.nih.gov/nuccore/LT962688). However, the wild-type strain of M. extorquens TK 0001 does not produce via HPLC or GC-MS measurable amounts of glycolic acid, despite the presence of the endogenous glyoxylate reductase DNA sequence and glyoxylate as the starting compound in the metabolism.

It is desirable to provide a fermentative production of glycolic acid from Cx compounds such as methanol or formic acid and the means thereto, in particular methylotrophic microorganisms that are able to convert such Cx compounds, for example methanol, formic acid or a mixture thereof, into glycolic acid. It is also desirable to provide a process by which glycolic acid can be recovered in a completely renewable way via an integrated process cascade from CO₂ as the only raw material, thus without the consumption of fossil or biogenic resources.

The technical problem underlying the present invention is therefore to overcome the disadvantages mentioned above. In particular, the technical problem underlying the present invention is to provide a biological cell that makes it possible to convert a starting material, hereinafter also referred to as a reactant, containing at least one Cx compound, in particular methanol, formic acid or a mixture thereof, into a product containing glycolic acid. In particular, the technical problem underlying the present invention is to provide means and processes that enable such a cell to be obtained, in particular means and processes that are cost-effective and easy to handle. In particular, the technical problem underlying the present invention is to provide means and processes, in particular a cost-effective and easy-to-handle process, for obtaining a product containing glycolic acid. In particular, the technical problem underlying the present invention is to provide means and processes that enable a sustainable synthesis of glycolic acid, which is almost completely without, in particular without, the use of fossil resources and/or almost completely without, in particular without, biogenic raw materials, and preferably starting from CO₂ as the only raw material. In particular, the technical problem of the present invention is to provide such means and processes that are cost-effective, environmentally friendly and easy to handle. Furthermore, in particular the technical problem underlying the present invention is to provide means and processes that enable the obtaining of polyglycolic acid, polylactic acid or polylactide-co-glycolide.

The technical problem is solved by the teachings of the independent claims, the dependent claims and the teaching of the description, in particular by a genetically modified Methylobacteriaceae cell comprising at least one exogenous nucleic acid sequence encoding a glyoxylate reductase from the bacterium Escherichia.

According to the invention, it is thus envisaged to provide a genetically modified Methylobacteriaceae cell, thus a cell that differs from the Methylobacteriaceae wild-type strain by at least one genetic modification. Further, according to the invention it is provided that the genetically modified Methylobacteriaceae cell comprises at least one exogenous nucleic acid sequence, wherein the nucleic acid sequence encodes a glyoxylate reductase from the bacterium Escherichia. The genetic modification of the wild-type strain of the Methylobacteriaceae cell is accordingly at least the genetic integration of at least one exogenous nucleic acid sequence into the Methylobacteriaceae cell, wherein the exogenous nucleic acid sequence encodes a glyoxylate reductase from the bacterium Escherichia. The exogenous nucleic acid sequence may be of synthetic origin or naturally occurring, in particular in Escherichia. In a preferred embodiment of the present invention, the genetically modified Methylobacteriaceae cell according to the invention comprises at least one exogenous nucleic acid sequence encoding a glyoxylate reductase, which is naturally occurring or is a codon-optimised, in particular Methylobacteriaceae-codon-optimised, in particular Methylorubrum-codon-optimised or Methylobacterium-codon-optimised, in particular Methylorubrum extorquens-codon-optimised, in particular Methylorubrum extorquens TK 0001-, Methylorubrum extorquens PA1- or Methylorubrum AM1-codon-optimised nucleic acid sequence.

Surprisingly, such a genetically modified Methylobacteriaceae cell according to the invention enables a Cx compound to be converted into glycolic acid, in particular to convert a starting material, namely a reactant containing at least one Cx compound, in particular methanol, formic acid or a mixture thereof, into a product containing glycolic acid, in particular to via HPLC or GC-MS measurable amounts. In contrast, the wild-type strain of the Methylobacteriaceae cell, which has only one endogenous nucleic acid sequence encoding a glyoxylate reductase, is not able to convert a Cx compound into glycolic acid, in particular to convert the starting material, namely a reactant containing at least one Cx compound, into a product containing glycolic acid, in particular to via HPLC or GC-MS measurable amounts.

The present invention thus provides a genetically modified Methylobacteriaceae cell that is able to convert a reactant containing at least one Cx compound, in particular methanol, formic acid or a mixture thereof, into a product containing glycolic acid. Cx compounds advantageously represent renewable but non-biogenic substrates for biotechnological processes, which, in addition, are easy to handle due to their liquid state and, unlike gases, are not limited in mass transfer in liquid reaction mixtures and are particularly easily made accessible by the invention for glycolic acid production. Glycolic acid can thus be produced from CO₂ in a completely renewable way, as long as the CO₂ conversion to a Cx compound, in particular methanol, is carried out with renewable energy. In this way, PtX (Power-to-X) processes can be advantageously connected with biotechnology to form an exemplary PtXtY (Power-to-X-to-Y) process.

Accordingly, the genetically modified Methylobacteriaceae cell according to the invention can advantageously be used in a process for producing glycolic acid from at least one Cx compound, in particular for producing a product containing glycolic acid, by converting a reactant containing at least one Cx compound, in particular methanol, formic acid or a mixture thereof. Preferably, the product containing glycolic acid obtained by the genetically modified Methylobacteriaceae cell according to the invention additionally has lactic acid besides glycolic acid. According to the invention, a particularly simple, easy-to-handle and cost-effective producing process for a product containing glycolic acid, in particular glycolic acid and lactic acid, is provided, so that a high level of equipment and cost-related effort is avoided. The present invention is also advantageous in that it enables the polymerisation of glycolic acid, in particular glycolic acid and lactic acid, for the production of polyglycolic acid, in particular polyglycolic acid, polylactic acid or polylactide-co-glycolide, which is often subsequently carried out after the provision of glycolic acid, in particular glycolic acid and lactic acid and accordingly enables the preparation of polyglycolic acid, in particular polyglycolic acid, polylactic acid or polylactide-co-glycolide, without the need to carry out cost-intensive and extensive process steps.

Without wanting to be bound by the theory, due to the exogenous glyoxylate reductase present in the genetically modified Methylobacteriaceae cell according to the invention, encoded by the at least one exogenous nucleic acid sequence encoding a glyoxylate reductase from the bacterium Escherichia, in the serine cycle of the genetically modified Methylobacteriaceae cell according to the invention it is possible to converted a Cx compound into glycolic acid, in particular to convert a reactant containing at least one Cx compound, in particular methanol, formic acid or a mixture thereof, into a product containing glycolic acid, in particular via HPLC or GC-MS measurable amounts. Preferably, the glyoxylate reductase encoded endogenously in the wild-type strain of the genetically modified Methylobacteriaceae cell according to the invention does not convert in the metabolism of the wild-type strain any via HPLC or GC-MS measurable amounts, in particular any, of a reactant containing at least one Cx compound, in particular methanol, formic acid or a mixture thereof, into a product containing glycolic acid, so that the glyoxylate reductase activity is solely controllable by the integration provided according to the invention of the exogenous glyoxylate reductase encoding nucleic acid sequence in genomic or episomal form and its expression.

The genetically modified Methylobacteriaceae cell according to the invention is thus characterised by the enzymatic activity of the exogenous glyoxylate reductase, in particular its ability, due to the presence of the exogenous glyoxylate reductase, to convert a Cx compound into glycolic acid, in particular a reactant containing at least one Cx compound, in particular methanol, formic acid or a mixture thereof, in particular in a reaction medium, into a product containing glycolic acid, in particular in a liquid reaction medium, in particular to catalysing this reaction enzymatically.

The genetically modified Methylobacteriaceae cell is preferably characterised in that it, in particular its genome, resembles the wild-type strain of the Methylobacteriaceae cell, in particular is identical to it, except for the presence of at least one exogenous nucleic acid sequence encoding a glyoxylate reductase from the bacterium Escherichia, which gives the Methylobacteriaceae cell according to the invention the enzymatic activity advantageous according to the invention, and optionally exogenous nucleic acid sequences of an expression vector or an expression cassette connected thereto. In a preferred embodiment of the present invention, in a Methylobacteriaceae according to the invention, in particular its genome, in addition to the at least one nucleic acid sequence encoding the glyoxylate reductase, further, in particular genetically engineered, genetic modifications may be present in comparison to the wild-type strain.

In a preferred embodiment of the present invention, the bacterium is Escherichia coli, in particular E. coli K-12 MG1655.

In a preferred embodiment of the present invention, the Methylobacteriaceae cell is a Methylorubrum cell, in particular a cell of Methylorubrum extorquens, in particular Methylorubrum extorquens TK 0001, in particular Methylorubrum extorquens PA1, Methylorubrum extorquens AM1, Methylorubrum rhodesianum or Methylorubrum zatmanii.

In a preferred embodiment of the present invention, the genetically modified Methylobacteriaceae cell according to the invention is a genetically modified Methylorubrum extorquens AM1 cell, a genetically modified Methylorubrum extorquens TK 0001 cell, or a genetically modified Methylorubrum extorquens PA1 cell comprising at least one exogenous nucleic acid sequence encoding a glyoxylate reductase from a bacterium Escherichia coli, in particular E. coli K-12 MG1655.

In a preferred embodiment of the present invention, the Methylobacteriaceae cell is a Methylobacterium cell, in particular a cell of Methylobacterium organophilum or Methylobacterium radiotolerans.

In a preferred embodiment of the present invention, the Methylobacteriaceae cell is a Methylorubrum cell, in particular a cell of Methylorubrum extorquens, in particular Methylorubrum extorquens AM1, Methylorubrum extorquens TK 0001, Methylorubrum extorquens PA1, Methylorubrum rhodesianum or Methylorubrum zatmanii, or a Methylobacterium cell, in particular a cell of Methylobacterium organophilum or Methylobacterium radiotolerans.

In a preferred embodiment of the present invention, the exogenous glyoxylate reductase is encoded by a nucleic acid sequence according to SEQ ID NO: 3 or a functional nucleic acid sequence equivalent thereof, wherein the functional nucleic acid sequence equivalent has a nucleic acid sequence identity of at least 30.0%, preferably 30.0 to 99.9%, preferably 40.0 to 99.9%, preferably 50.0 to 99.9%, preferably 60.0 to 99.9%, preferably 70.0 to 99.9%, preferably from 76.0 to 99.9%, preferably from 80.0 to 99.9%, preferably 90.0 to 99.9%, preferably 95.0 to 99.9%, preferably 98.0 to 99.9%, preferably 90.0 to 99.0% to the nucleic acid sequence according to SEQ ID NO: 3 and wherein the glyoxylate reductase encoded thereby is able to convert a reactant containing at least one Cx compound, in particular methanol, formic acid or a mixture thereof, into a product containing glycolic acid. Preferably, the nucleic acid sequence identity is at least 76.0 to the nucleic acid sequence according to SEQ ID NO: 3.

In a preferred embodiment of the present invention, the present invention thus relates to a genetically modified Methylobacteriaceae cell, in particular a Methylorubrum cell or Methylobacterium cell, comprising a nucleic acid sequence encoding an exogenous glyoxylate reductase, in particular a nucleic acid sequence according to SEQ ID NO: 3.

In a preferred embodiment of the present invention, the present invention also relates to a genetically modified Methylobacteriaceae cell, in particular a Methylorubrum cell or Methylobacterium cell, comprising a functional nucleic acid sequence equivalent of at least one exogenous nucleic acid sequence encoding a glyoxylate reductase according to SEQ ID NO: 3, wherein the functional nucleic acid sequence equivalent has a nucleic acid sequence identity of at least 30.0%, preferably 30.0 to 99.9%, preferably 40.0 to 99.9%, preferably 50.0 to 99.9%, preferably 60.0 to 99.9%, preferably 70.0 to 99.9%, preferably 76.0 to 99.9%, preferably 80.0 to 99.9%, preferably 90.0 to 99.9%, preferably 95.0 to 99.9%, preferably 98.0 to 99.9%, preferably 90.0 to 99.0%, to the nucleic acid sequence according to SEQ ID NO: 3 and wherein the glyoxylate reductase encoded thereby is able to convert a reactant containing at least one Cx compound, in particular methanol, formic acid or a mixture thereof, into a product containing glycolic acid.

In a particularly preferred embodiment of the present invention, the functional nucleic acid sequence equivalent of the nucleic acid sequence according to SEQ ID NO: 3 has a nucleic acid sequence with a length of at least 800, preferably at least 850, preferably at least 900, preferably at least 950, preferably at least 970, nucleic acids. Preferably according to the invention, the sequence identity of the nucleic acid sequence of the nucleic acid sequence equivalent of the nucleic acid sequence according to SEQ ID NO: 3 to the nucleic acid sequence according to SEQ ID NO: 3 is given over the entire length of the nucleic acid sequence of the nucleic acid sequence equivalent of the nucleic acid sequence according to SEQ ID NO: 3.

In a particularly preferred embodiment of the present invention, the nucleic acid sequence according to SEQ ID NO: 3 is a codon-optimised, in particular a Methylorubrum-, in particular Methylorubrum extorquens-, in particular Methylorubrum extorquens AM1, Methylorubrum extorquens TK 0001-, in particular a Methylorubrum extorquens PA1 codon-optimised nucleic acid sequence of the native, thus naturally occurring, nucleic acid sequence from Escherichia, in particular E. coli, which encodes the glyoxylate reductase from Escherichia, in particular E. coli. The native nucleic acid sequence from Escherichia encoding the glyoxylate reductase from Escherichia has the nucleic acid sequence according to SEQ ID NO: 1 and is a functional nucleic acid sequence equivalent of the nucleic acid sequence according to SEQ ID NO: 3.

In a particularly preferred embodiment of the present invention, the functional nucleic acid sequence equivalent of the nucleic acid sequence according to SEQ ID NO: 3 has the nucleic acid sequence according to SEQ ID NO: 1.

In a preferred embodiment of the present invention, the exogenous nucleic acid sequence encoding a glyoxylate reductase from the bacterium Escherichia, in particular according to SEQ ID NO: 3 or a functional nucleic acid sequence equivalent thereof, for example according to SEQ ID NO: 1, encodes a glyoxylate reductase comprising, in particular consisting of, an amino acid sequence according to SEQ ID NO: 2 or a functional amino acid sequence equivalent thereof.

In a preferred embodiment of the present invention, the exogenous glyoxylate reductase has an amino acid sequence according to SEQ ID NO: 2 or a functional amino acid sequence equivalent thereof, wherein the functional amino acid sequence equivalent has an amino acid sequence identity of at least 30.0%, in particular 30.0 to 99.9%, preferably 40.0 to 99.9%, preferably 50.0 to 99.9%, preferably 60.0 to 99.9%, preferably 70.0 to 99.9%, preferably from 76.0 to 99.9%, preferably from 80.0 to 99.9%, preferably 85.0 to 99.9%, preferably 90.0 to 99.9%, preferably 95.0 to 99.9%, preferably 98.0 to 99.9%, to the amino acid sequence according to SEQ ID NO: 2. Preferably, the amino acid sequence identity is at least 90.0% to the amino acid sequence according to SEQ ID NO: 2.

In a preferred embodiment of the present invention, the present invention relates to a genetically modified Methylobacteriaceae cell, in particular a Methylorubrum cell or a Methylobacterium cell, comprising a functional amino acid sequence equivalent of the amino acid sequence of SEQ ID NO: 2, wherein the functional amino acid sequence equivalent has an amino acid sequence identity of at least 30.0%, in particular 30.0 to 99.9%, preferably 40.0 to 99.9%, preferably 50.0 to 99.9%, preferably 60.0 to 99.9%, preferably 70.0 to 99.9%, preferably 76.0 to 99.9%, preferably 80.0 to 99.9%, preferably 85.0 to 99.9%, preferably 90.0 to 99.9%, preferably 95.0 to 99.9%, preferably 98.0 to 99.9%, to the amino acid sequence according to SEQ ID NO: 2 and which is able to convert a reactant containing at least one Cx compound, in particular methanol, formic acid or a mixture thereof, into a product containing glycolic acid.

In a particularly preferred embodiment of the present invention, the functional amino acid sequence equivalent of the amino acid sequence according to SEQ ID NO: 2 has an amino acid sequence with a length of at least 300, preferably at least 310, preferably at least 320, preferably at least 325 amino acids. Preferably according to the invention, the sequence identity of the amino acid sequence of the amino acid sequence equivalent of the amino acid sequence according to SEQ ID NO: 2 to the amino acid sequence according to SEQ ID NO: 2 is given over the entire length of the amino acid sequence of the amino acid sequence equivalent of the amino acid sequence according to SEQ ID NO: 2.

In a preferred embodiment of the present invention, the Cx compound is a Cx compound with x=1, 2 or 4, in particular x=1.

In a preferred embodiment of the present invention, the Cx compound is formic acid, methanol, methane, methylamine, acetic acid or succinic acid or a mixture thereof.

In a preferred embodiment of the present invention, the Cx compound is methanol.

In a preferred embodiment of the present invention, the Cx compound is formic acid.

In a preferred embodiment of the present invention, the reactant contains at least one Cx compound, in particular formic acid, methanol, methane, methylamine, acetic acid or succinic acid or a mixture thereof, in particular the reactant consists of at least one compound thereof. In a preferred embodiment of the present invention, the product obtained by converting a reactant containing at least one Cx compound contains glycolic acid, in particular consists of it.

In a preferred embodiment of the present invention, the product obtained by converting a reactant containing at least one Cx compound contains glycolic acid and lactic acid, in particular consists of these.

In a preferred embodiment of the present invention, the product containing glycolic acid contains glycolic acid and lactic acid, in particular comprises 1 to 99 wt. %, in particular 2 to 98 wt. %, in particular 10 to 90 wt. %, in particular 30 to 80 wt. %, in particular 40 to 70 wt. %, in particular 50 wt. %, in particular 60 wt. %, glycolic acid and in particular 1 to 99 wt. %, in particular 2 to 98 wt. %, in particular 10 to 90 wt. %, in particular 20 to 70 wt. %, in particular 30 to 60 wt. %, in particular 50 wt. %, in particular 40 wt. %, lactic acid (each based on total dry weight of the obtained product) or consists of these proportions.

In a preferred embodiment of the present invention, the growth rate μₘₐₓ of a genetically modified Methylobacteriaceae cell according to the invention, in particular in a reaction medium having an initial concentration of up to 10 g L⁻¹ of a reactant containing at least one Cx compound, in particular consisting of methanol, is at least 0.05 h⁻¹, at least 0.10 h⁻¹, in particular at least 0.15 h⁻¹, in particular at least 0.18 h⁻¹, in particular at least 0.20 h⁻¹, in particular at least 0.21 h⁻¹, in particular 0.10 to 0.30 h⁻¹, in particular 0.15 to 0.25 h⁻¹, in particular 0.20 to 0.22 h⁻¹, in particular 0.21 h⁻¹.

In a preferred embodiment of the present invention, the titer of a reaction medium containing the product containing glycolic acid, in particular glycolic acid and lactic acid, which is obtained after converting a reactant containing at least one Cx compound, in particular consisting of methanol, by a genetically modified Methylobacteriaceae cell according to the invention, in a reaction medium having an initial concentration of up to 10 g L⁻¹ reactant, in particular after a reaction time of 40 h, is at least 0.01 g L⁻¹, at least 0.10 g L⁻¹, in particular at least 0.15 g L⁻¹, in particular at least 0.20 g L⁻¹, in particular at least 0.25 g L⁻¹, in particular at least 0.50 g L⁻¹, in particular at least 0.75 g L⁻¹, in particular at least 1.00 g L⁻¹ and in particular 1.50 g L⁻¹ (each based on weight of the product per litre of reaction medium).

In a preferred embodiment of the present invention, a genetically modified Methylobacteriaceae cell according to the invention converts a reactant containing at least one Cx compound, in particular consisting of methanol, into a product containing glycolic acid, in particular glycolic acid and lactic acid, in particular in a reaction medium having an initial concentration of up to 10 g L⁻¹ of the reactant with a dry-biomass-substrate-yield (Y_{X/S}) of at least 10 mg g⁻¹, in particular at least 50 mg g⁻¹, in particular at least 100 mg g⁻¹, in particular at least 150 mg g⁻¹, in particular at least 200 mg g⁻¹, in particular 10 to 350 mg g⁻¹, in particular 50 to 320 mg g⁻¹, in particular 100 to 300 mg g⁻¹, in particular 200 to 300 mg g⁻¹, in particular 280 mg g⁻¹ (each based on dry biomass of the genetically modified Methylobacteriaceae cell according to the invention per gram of the reactant).

In a preferred embodiment of the present invention, the dry-biomass-substrate-yield (Y_{X/S}) of a genetically modified Methylobacteriaceae cell according to the invention decreases in relation to the dry-biomass-substrate-yield (Y_{X/S}) of the wild-type strain when converting a reactant containing at least one Cx compound, in particular consisting of methanol, into a product containing glycolic acid, in particular glycolic acid and lactic acid, in particular in a reaction medium having an initial concentration of up to 10 g L⁻¹ of the reactant to less than 95%, in particular less than 90%, in particular less than 80%, in particular less than 70%, in particular 68%.

In a preferred embodiment of the present invention, a genetically modified Methylobacteriaceae cell according to the invention converts a reactant containing at least one Cx compound, in particular consisting of methanol, into a product containing glycolic acid, in particular glycolic acid and lactic acid, in particular in a reaction medium having an initial concentration of up to 10 g L⁻¹ of the reactant, with a product-substrate-yield (Y_{P/S}) of at least 10 mg g⁻¹, in particular at least 50 mg g⁻¹, in particular at least 80 mg g⁻¹, in particular at least 100 mg g⁻¹, in particular at least 110 mg g⁻¹, in particular 10 to 200 mg g⁻¹, in particular 50 to 180 mg g⁻¹, in particular 80 to 150 mg g⁻¹, in particular 100 to 130 mg g⁻¹, in particular 120 mg g⁻¹ (each based on weight of the product per gram of reactant).

In a preferred embodiment of the present invention, a genetically modified Methylobacteriaceae cell according to the invention converts a reactant containing at least one Cx compound, in particular consisting of methanol, into a product containing glycolic acid, in particular glycolic acid and lactic acid, in particular in a reaction medium having an initial concentration of up to 10 g L⁻¹ of the reactant, with a product-dry-biomass-yield (Y_{P/X}) of at least 0.10 g g⁻¹, in particular at least 0.20 g g⁻¹, in particular at least 0.30 g g⁻¹, in particular at least 0.40 g g⁻¹, in particular at least 0.50 g g⁻¹, in particular 0.10 to 0.80 g g⁻¹, in particular 0.20 to 0.70 g g⁻¹, in particular 0.30 to 0.60 g g⁻¹, in particular 0.40 to 0.50 g g⁻¹, in particular 0.50 g g⁻¹ (each based on weight of the product per gram of dry biomass of the genetically modified Methylobacteriaceae cell according to the invention).

In a preferred embodiment of the present invention, the genetically modified Methylobacteriaceae cell according to the invention comprises at least one exogenous nucleic acid sequence encoding an ethylmalonyl-CoA mutase.

In a preferred embodiment of the present invention, the genetically modified Methylobacteriaceae cell according to the invention comprises at least one exogenous nucleic acid sequence encoding an ethylmalonyl-CoA mutase which is naturally occurring or a codon-optimised, in particular Methylobacteriaceae-codon-optimised, nucleic acid sequence, in particular a Methylobacterium-, in particular Methylorubrum-, in particular Methylorubrum extorquens-, in particular Methylorubrum extorquens AM1, Methylorubrum extorquens TK 0001- or Methylorubrum extorquens PA1-codon-optimised nucleic acid sequence.

In a preferred embodiment of the present invention, the exogenous nucleic acid sequence encoding an ethylmalonyl-CoA mutase is derived in particular from at least one bacterium selected from the group consisting of Methylorubrum extorquens, in particular Methylorubrum extorquens TK 0001 DSM 1337, and Rhodobacter sphaeroides, in particular Rhodobacter sphaeroides ATCC 17029.

In a preferred embodiment of the present invention, the genetically modified Methylobacteriaceae cell according to the invention is a genetically modified Methylobacteriaceae cell, in particular a Methylorubrum extorquens AM1, Methylorubrum extorquens PA1, Methylorubrum extorquens TK 0001 cell, comprising at least one exogenous nucleic acid sequence encoding a glyoxylate reductase from a bacterium Escherichia coli, in particular E. coli K-12 MG1655, and at least one exogenous nucleic acid sequence encoding an ethylmalonyl-CoA.

In a preferred embodiment of the present invention, the genetically modified Methylobacteriaceae cell according to the invention is a genetically modified Methylobacteriaceae cell, in particular a Methylorubrum extorquens AM1, Methylorubrum extorquens PA1, Methylorubrum extorquens TK 0001 cell, comprising at least one exogenous nucleic acid sequence encoding a glyoxylate reductase from a bacterium Escherichia coli K-12 MG1655 and at least one exogenous nucleic acid sequence encoding an ethylmalonyl-CoA mutase from a bacterium Methylorubrum extorquens TK 0001 DSM 1337.

In a preferred embodiment of the present invention, a genetically modified Methylobacteriaceae cell according to the present invention is a genetically modified Methylobacteriaceae cell, in particular a Methylorubrum extorquens AM1, Methylorubrum extorquens PA1, Methylorubrum extorquens TK 0001 cell comprising at least one exogenous nucleic acid sequence encoding a glyoxylate reductase from a bacterium Escherichia coli K-12 MG1655 and at least one exogenous nucleic acid sequence encoding an ethylmalonyl-CoA mutase from a bacterium Rhodobacter sphaeroides ATCC 17029.

In a preferred embodiment of the present invention, the present invention also relates to a genetically modified Methylobacteriaceae cell comprising at least two different exogenous nucleic acid sequences, that is, a genetically modified Methylobacteriaceae cell which comprises besides the at least one exogenous nucleic acid sequence encoding a glyoxylate reductase from the bacterium Escherichia at least one additionally exogenous nucleic acid sequence, that encodes an ethylmalonyl-CoA mutase.

Surprisingly, such a genetically modified Methylobacteriaceae cell according to the invention enables an increased glycolic acid yield, in particular a glycolic acid and lactic acid yield, compared to the glycolic acid yield, in particular glycolic acid and lactic acid yield, obtained by converting a reactant containing at least one Cx compound, in particular methanol, formic acid or a mixture thereof, by a Methylobacteriaceae cell according to the invention, comprising at least one exogenous nucleic acid sequence encoding a glyoxylate reductase from the bacterium Escherichia, which has no exogenous nucleic acid sequence encoding an ethylmalonyl-CoA mutase, in particular no encoding nucleic acid from at least one bacterium selected from the group consisting of Methylorubrum extorquens, in particular Methylorubrum extorquens TK 0001 DSM 1337, and Rhodobacter sphaeroides, in particular Rhodobacter sphaeroides ATCC 17029. The invention therefore surprisingly increases not only the glycolic acid yield, but also the yield of lactic acid.

Preferably, the conversion of Cx compounds into glycolic acid, in particular glycolic acid and lactic acid, by a Methylobacteriaceae cell according to the invention, additionally comprising the at least one exogenous nucleic acid sequence encoding an ethylmalonyl-CoA mutase, in particular from at least one bacterium selected from the group consisting of Methylorubrum extorquens, in particular Methylorubrum extorquens TK 0001 DSM 1337, and Rhodobacter sphaeroides, in particular Rhodobacter sphaeroides ATCC 17029, is higher than the conversion by a Methylorubrum cell according to the invention without this at least one additionally exogenous nucleic acid sequence.

Without wanting to be bound by theory, due to the exogenous ethylmalonyl-CoA mutase present in the genetically modified Methylobacteriaceae cell according to the invention the amount of glyoxylate in the serine cycle of the genetically modified Methylobacteriaceae cell according to the invention is increased, which is converted by the exogenous glyoxylate reductase present in the genetically modified Methylobacteriaceae cell according to the invention. This leads preferably to an improved glycolic acid yield compared to the glycolic acid yield obtained by a Methylobacteriaceae cell according to the invention comprising at least one exogenous glyoxylate reductase from the bacterium Escherichia, which has no exogenous nucleic acid sequence encoding an ethylmalonyl-CoA mutase. The increased lactic acid yield also observed is possibly due, without being bound to the theory, to a complex interaction with the metabolism of a reduction equivalent provision and an increased availability of the metabolite pyruvate, the precursor molecule of lactic acid.

In a particularly preferred embodiment, the present invention relates to a genetically modified Methylobacteriaceae cell according to the invention, comprising a codon-optimised nucleic acid sequence of a nucleic acid sequence from Rhodobacter sphaeroides encoding an ethylmalonyl-CoA mutase, in particular a Methylobacteriaceae, in particular Methylobacterium, in particular a Methylorubrum-, in particular Methylorubrum extorquens-, in particular Methylorubrum extorquens TK 0001, in particular Methylorubrum extorquens AM1, in particular Methylorubrum extorquens PA1, codon-optimised nucleic acid sequence, in particular it has the SEQ ID NO: 8.

In a particularly preferred embodiment, the present invention relates to a genetically modified Methylobacteriaceae cell according to the invention comprising a codon-optimised nucleic acid sequence of a nucleic acid sequence from Methylorubrum extorquens encoding an ethylmalonyl-CoA mutase, in particular a Methylobacteriaceae, in particular Methylobacterium-, in particular Methylorubrum-, in particular Methylorubrum extorquens-, in particular Methylorubrum extorquens TK 0001, in particular Methylorubrum extorquens AM1, in particular Methylorubrum extorquens PA1-, codon-optimised nucleic acid sequence, in particular it has SEQ ID NO: 13.

In a preferred embodiment, the present invention relates to a genetically modified Methylobacteriaceae cell according to the invention comprising a functional nucleic acid sequence equivalent of a nucleic acid sequence encoding an ethylmalonyl-CoA mutase according to SEQ ID NO: 8 or 13.

The native nucleic acid sequences of the ethylmalonyl-CoA mutase from Methylorubrum or Rhodobacter according to SEQ ID NO: 4 and 6 are also understood in the context of the present invention as functional equivalents of the codon-optimised nucleic acid sequences derived therefrom, in particular the native nucleic acid sequence according to SEQ ID NO: 6 is a functional nucleic acid sequence equivalent of the codon-optimised nucleic acid sequence according to SEQ ID NO: 8 and the native nucleic acid sequence according to SEQ ID NO: 4 is a functional nucleic acid sequence equivalent of the codon-optimised nucleic acid sequence according to SEQ ID NO: 13.

In a preferred embodiment of the present invention, the ethylmalonyl-CoA mutase is encoded by a codon-optimised nucleic acid sequence (SEQ ID NO: 13 or 8) of a native nucleic acid sequence according to SEQ ID NO: 4 or 6 or a functional equivalent thereof, in particular the native nucleic acid sequence itself, thus a nucleic acid sequence according to SEQ ID NO: 4 or 6, wherein the functional nucleic acid sequence equivalent has a nucleic acid sequence identity of at least 30.0%, preferably 30.0 to 99.9%, preferably 40.0 to 99.9%, preferably 50.0 to 99.9%, preferably 60.0 to 99.9%, preferably 70.0 to 99.9%, preferably 76.0 to 99.9%, preferably 80.0 to 99.9%, preferably 90.0 to 99.9%, preferably 95.0 to 99.9%, preferably 98.0 to 99.9%, preferably 90.0 to 99.0%, to the codon-optimised nucleic acid sequence according to SEQ ID NO: 13 or 8, wherein the functional equivalent has the enzymatic activity of an ethylmalonyl-CoA mutase.

In a preferred embodiment of the present invention, the present invention also relates to a genetically modified Methylobacteriaceae cell according to the invention comprising a functional nucleic acid sequence equivalent of the at least one exogenous, codon-optimised nucleic acid sequence encoding an ethylmalonyl-CoA mutase according to SEQ ID NO: 13 or 8, thus, for example a native nucleic acid sequence according to SEQ ID NO: 4 or 6, wherein the functional nucleic acid sequence equivalent has a nucleic acid sequence identity of at least 30.0%, preferably 30.0 to 99.9%, preferably 40.0 to 99.9%, preferably 50.0 to 99.9%, preferably 60.0 to 99.9%, preferably 70.0 to 99.9%, preferably 76.0 to 99.9%, preferably 80.0 to 99.9%, preferably 90.0 to 99.9%, preferably 95.0 to 99.9%, preferably 98.0 to 99.9%, preferably 90.0 to 99.0%, to the codon-optimised nucleic acid sequence according to SEQ ID NO: 13 or 8, and wherein the modified Methylobacteriaceae cell is able to convert a reactant containing at least one Cx compound, in particular methanol, formic acid or a mixture thereof, into a product containing glycolic acid.

In a particularly preferred embodiment of the present invention, the functional nucleic acid sequence equivalent of the codon-optimised nucleic acid sequence according to SEQ ID NO: 8 has the native nucleic acid sequence according to SEQ ID NO: 6.

In a particularly preferred embodiment of the present invention, the functional nucleic acid sequence equivalent of the codon-optimised nucleic acid sequence according to SEQ ID NO: 13 has the native nucleic acid sequence according to SEQ ID NO: 4.

In a preferred embodiment of the present invention, the ethylmalonyl-CoA mutase has an amino acid sequence according to SEQ ID NO: 5 or 7 or a functional equivalent thereof, wherein the functional amino acid sequence equivalent has an amino acid sequence identity of at least 30.0%, in particular 30.0 to 99.9%, preferably 40.0 to 99.9%, preferably 50.0 to 99.9%, preferably 60.0 to 99.9%, preferably 70.0 to 99.9%, preferably 76.0 to 99.9%, preferably 80.0 to 99.9%, preferably 85.0 to 99.9%, preferably 90.0 to 99.9%, preferably 95.0 to 99.9%, preferably 98.0 to 99.9%, to the amino acid sequence according to SEQ ID NO: 5 or 7 and has the enzymatic activity of an ethylmalonyl-CoA mutase.

In a preferred embodiment of the present invention, the present invention relates to a genetically modified Methylobacteriaceae cell according to the invention comprising a functional amino acid sequence equivalent of the amino acid sequence of SEQ ID NO: 5 or 7, wherein the functional amino acid sequence equivalent has an sequence identity of at least 30.0%, in particular 30.0 to 99.9%, preferably 40.0 to 99.9%, preferably 50.0 to 99.9%, preferably 60.0 to 99.9%, preferably 70.0 to 99.9%, preferably 76.0 to 99.9%, preferably 80.0 to 99.9%, preferably 85.0 to 99.9%, preferably 90.0 to 99.9%, preferably 95.0 to 99.9%, preferably 98.0 to 99.9%, to the amino acid sequence according to SEQ ID NO: 5 or 7 and wherein the modified Methylobacteriaceae cell is able to convert a reactant containing at least one Cx compound, in particular methanol, formic acid or a mixture thereof, into a product containing glycolic acid, in particular glycolic acid and lactic acid.

In a preferred embodiment of the present invention, the growth rate μₘₐₓ of a genetically modified Methylobacteriaceae cell according to the invention, additionally comprising at least one exogenous nucleic acid sequence encoding an ethylmalonyl-CoA mutase, in a reaction medium having an initial concentration of up to 10 g L⁻¹, in particular 10 g L⁻¹, of a reactant containing at least one Cx compound, in particular consisting of methanol, is at least 0.05 h⁻¹, at least 0.10 h⁻¹, in particular at least 0.12 h⁻¹, in particular at least 0.14 h⁻¹, in particular at least 0.16 h⁻¹, in particular 0.10 to 0.25 h⁻¹, in particular 0.12 to 0.22 h⁻¹, in particular 0.15 to 0.20 h⁻¹, in particular 0.16 h⁻¹, in particular 0.19 h⁻¹.

In a preferred embodiment of the present invention, the titer of a reaction medium containing the product containing glycolic acid, in particular glycolic acid and lactic acid, which is obtained after converting a reactant containing at least one Cx compound, in particular consisting of methanol, by a genetically modified Methylobacteriaceae cell according to the invention, additionally comprising at least one exogenous nucleic acid sequence encoding an ethylmalonyl-CoA mutase, in particular in a reaction medium having an initial concentration of up to 10 g L⁻¹, in particular 10 g L⁻¹, of the reactant, in particular after a reaction time of 40 h, is at least 0.10 g L⁻¹, in particular at least 0.20 g L⁻¹, in particular at least 0.30 g L⁻¹, in particular at least 0.40 g L⁻¹, in particular 0.10 to 80 g L⁻¹, in particular 0.20 to 70 g L⁻¹, in particular 0.30 to 60 g L⁻¹, in particular 0.40 to 55 g L⁻¹, in particular 0.49 g L⁻¹, in particular 0.52 g L⁻¹ (each based on weight of the product per litre of reaction medium).

In a preferred embodiment of the present invention, the titer, which is obtained by converting a reactant containing at least one Cx compound, in particular consisting of methanol, by the genetically modified Methylobacteriaceae cell according to the invention, additionally comprising at least one exogenous nucleic acid sequence encoding an ethylmalonyl-CoA mutase encoding nucleic acid sequence, in a reaction medium having an initial concentration of up to 10 g L⁻¹ of the reactant, in particular after a reaction time of 40 h, compared to the titer obtained by the conversion by means of the genetically modified Methylobacteriaceae cell according to the invention without the at least one exogenous nucleic acid sequence encoding an ethylmalonyl-CoA mutase is increased by at least 10%, in particular at least 30%, in particular at least 50%, in particular at least 60%, in particular 69%, in particular 79%.

In a preferred embodiment of the present invention, a genetically modified Methylobacteriaceae cell according to the invention additionally comprising at least one exogenous nucleic acid sequence encoding an ethylmalonyl-CoA mutase converts a reactant containing at least one Cx compound, in particular consisting of methanol, into a product containing glycolic acid, in particular glycolic acid and lactic acid, in particular in a reaction medium having an initial concentration of up to 10 g L⁻¹, in particular 10 g L⁻¹, of the reactant with a dry-biomass-substrate-yield (Y_{X/S}) of at least 10 mg g⁻¹, in particular at least 50 mg g⁻¹, in particular at least 100 mg g⁻¹, in particular at least 150 mg g⁻¹, in particular at least 200 mg g⁻¹, in particular 10 to 350 mg g⁻¹, in particular 50 to 320 mg g⁻¹, in particular 100 to 300 mg g⁻¹, in particular 200 to 300 mg g⁻¹, in particular 210 mg g⁻¹, in particular 270 mg g⁻¹ (each based on dry biomass of the genetically modified Methylobacteriaceae cell according to the invention per gram of reactant).

In a preferred embodiment of the present invention, the dry-biomass-substrate-yield (Y_{X/S}) of a genetically modified Methylobacteriaceae cell according to the invention, additionally comprising at least one exogenous nucleic acid sequence encoding an ethylmalonyl-CoA decreases in relation to the biomass-substrate-yield (Y_{X/S}) of the wild-type strain when converting a reactant containing at least one Cx compound, in particular consisting of methanol, into a product containing glycolic acid, in particular glycolic acid and lactic acid, in particular in a reaction medium having an initial concentration of up to 10 g L⁻¹, in particular 10 g L⁻¹, of the reactant to less than 95%, in particular less than 90%, in particular less than 80%, in particular less than 70%, in particular 68%, in particular 51%.

In a preferred embodiment of the present invention, the biomass-substrate-yield (Y_{X/S}) of a genetically modified Methylobacteriaceae cell according to the invention additionally comprising at least one exogenous nucleic acid sequence encoding an ethylmalonyl-CoA mutase decreases in relation to the biomass substrate (Y_{X/S}) of the genetically modified Methylobacteriaceae cell according to the invention without the at least one exogenous nucleic acid sequence encoding an ethylmalonyl-CoA mutase when converting a reactant containing at least one Cx compound, in particular consisting of methanol, into a product containing glycolic acid, in particular glycolic acid and lactic acid, in particular in a reaction medium having an initial concentration of up to 10 g L⁻¹, in particular 10 g L⁻¹, of the reactant to less than 99%, in particular less than 97%, in particular to 96%, in particular to 75%.

In a preferred embodiment of the present invention, the biomass-substrate-yield (Y_{X/S}) of a genetically modified Methylobacteriaceae cell according to the invention additionally comprising at least one exogenous nucleic acid sequence encoding an ethylmalonyl-CoA mutase from the bacterium Rhodobacter sphaeroides, in particular Rhodobacter sphaeroides ATCC 17029, is increased in relation to the biomass-substrate yield (Y_{X/S}) of a genetically modified Methylobacteriaceae cell according to the invention comprising at least one exogenous nucleic acid sequence encoding an ethylmalonyl-CoA mutase from the bacterium Methylorubrum extorquens, in particular Methylorubrum extorquens TK 0001 DSM 1337 in the conversion of a reactant containing at least one Cx compound, in particular consisting of methanol, into a product containing glycolic acid, in particular glycolic acid and lactic acid, in particular in a reaction medium having an initial concentration of up to 10 g L⁻¹, in particular 10 g L⁻¹, of the reactant by at least 5%, in particular at least 10%, in particular at least 20%, in particular at least 25%, in particular 28%.

In a preferred embodiment of the present invention, the genetically modified Methylobacteriaceae cell according to the invention additionally comprising at least one exogenous nucleic acid sequence encoding an ethylmalonyl-CoA mutase, converts a reactant containing at least one Cx compound, in particular consisting of methanol, into a product containing glycolic acid, in particular glycolic acid and lactic acid, in particular in a reaction medium having an initial concentration of up to 10 g L⁻¹, in particular 10 g L⁻¹, of the reactant, with a product-substrate yield (Y_{P/S}) of at least 10 mg g⁻¹, in particular at least 50 mg g⁻¹, in particular at least 80 mg g⁻¹, in particular at least 100 mg g⁻¹, in particular at least 140 mg g⁻¹, in particular 10 to 250 mg g⁻¹, in particular 50 to 200 mg g⁻¹, in particular 80 to 180 mg g⁻¹, in particular 100 to 160 mg g⁻¹, in particular 150 mg g⁻¹ (based on weight of the product per gram of reactant).

In a preferred embodiment of the present invention, the product-substrate yield (Y_{P/S}) of a genetically modified Methylobacteriaceae cell according to the invention additionally comprising at least one exogenous nucleic acid sequence encoding an ethylmalonyl-CoA mutase increases in relation to the product-substrate yield (Y_{P/S}) of the genetically modified Methylobacteriaceae cell according to the invention without the at least one exogenous nucleic acid sequence encoding an ethylmalonyl-CoA mutase when converting a reactant containing at least one Cx compound, in particular consisting of methanol, into a product containing glycolic acid, in particular glycolic acid and lactic acid, in particular in a reaction medium having up to 10 g L⁻¹, in particular 10 g L⁻¹, of the reactant by at least 10%, in particular at least 15%, in particular at least 20%, in particular 25%.

In a preferred embodiment of the present invention, the genetically modified Methylobacteriaceae cell according to the invention additionally comprising at least one exogenous nucleic acid sequence encoding an ethylmalonyl-CoA mutase converts a reactant containing at least one Cx compound, in particular consisting of methanol, into a product containing glycolic acid, in particular glycolic acid and lactic acid, in particular in a reaction medium having an initial concentration of up to 10 g L⁻¹, in particular 10 g L⁻¹, of the reactant with a product-dry-biomass-yield (Y_{P/X}) of at least 0.10 g g⁻¹, in particular at least 0.30 g g⁻¹, in particular at least 0.40 g g⁻¹, in particular at least 0.50 g g⁻¹, in particular at least 0.60 g g⁻¹, in particular 0.10 to 0.99 g g⁻¹, in particular 0.30 to 0.90 g g⁻¹, in particular 0.40 to 0.80 g g⁻¹, in particular 0.50 to 0.75 g g⁻¹, in particular 0.70 g g⁻¹, in particular 0.71 g g⁻¹ (each based on weight of the product per gram of dry biomass of the genetically modified Methylobacteriaceae cell according to the invention).

In a preferred embodiment of the present invention, the product-biomass-yield (Y_{P/X}) of a genetically modified Methylobacteriaceae cell according to the invention additionally comprising at least one exogenous nucleic acid sequence encoding an ethylmalonyl-CoA mutase increases in relation to the product-biomass yield (Y_{P/X}) of the genetically modified Methylobacteriaceae cell according to the invention without the at least one exogenous nucleic acid sequence encoding an ethylmalonyl-CoA mutase when converting a reactant containing at least one Cx compound, in particular consisting of methanol, into a product containing glycolic acid, in particular glycolic acid and lactic acid, in particular in a reaction medium having an initial concentration of up to 10 g L⁻¹, in particular 10 g L⁻¹, of the reactant by at least 10%, in particular at least 20%, in particular at least 30%, in particular at least 35%, in particular 40%, in particular 42%.

In a preferred embodiment of the present invention, the Methylobacteriaceae cell is a cell of Methylorubrum extorquens, in particular Methylorubrum extorquens TK 0001 and in particular 20 Methylorubrum extorquens PA1.

In a preferred embodiment of the present invention, the at least one exogenous nucleic acid sequence encoding a glyoxylate reductase is integrated into the chromosome of the Methylobacteriaceae cell or is present extrachromosomally, in particular is present in the cell integrated in an episomal expression vector or minichromosome.

In a preferred embodiment of the present invention, the at least one exogenous nucleic acid sequence encoding a glyoxylate reductase is stably integrated into the chromosome of the Methylobacteriaceae cell or is stably present extrachromosomally.

In a preferred embodiment of the present invention, more than one copy, in particular 2, 3, 4, 5, 6 or more copies of the exogenous nucleic acid sequence encoding a glyoxylate reductase are present in the genome of the Methylobacteriaceae cell, preferably stably integrated into the chromosome, or, preferably stable, present extrachromosomally.

In a preferred embodiment of the present invention, the at least one exogenous nucleic acid sequence encoding an ethylmalonyl-CoA mutase is integrated into the chromosome of the Methylobacteriaceae cell or is present extrachromosomally, in particular is present in the cell integrated in an episomal expression vector or minichromosome.

In a preferred embodiment of the present invention, the at least one exogenous nucleic acid sequence encoding an ethylmalonyl-CoA mutase is stably integrated into the chromosome of the Methylobacteriaceae cell or is stably present extrachromosomally.

In a preferred embodiment of the present invention, more than one copy, in particular 2, 3, 4, 5, 6 or more copies of the exogenous nucleic acid sequence encoding an ethylmalonyl-CoA mutase are present in the genome of the Methylobacteriaceae cell, preferably stably integrated into the chromosome, or, preferably stably, present extrachromosomally.

In a preferred embodiment of the present invention, the genetically modified Methylobacteriaceae cell is the Methylorubrum cell Methylorubrum extorquens Mea-GA1, Methylorubrum extorquens Mea-GA2 or Methylorubrum extorquens Mea-GA3, each deposited on 10 Jun. 2022 at the DSMZ, German Collection of Microorganisms and Cell Cultures, Braunschweig, Germany under the deposit numbers DSM 34286, DSM 34287 and DSM 34288. All deposits were made in accordance with the Budapest Treaty on the International Recognition of the Deposit of Microorganisms for the Purposes of Patent Procedure.

In a preferred embodiment of the present invention, the present invention relates to a genetically modified Methylorubrum extorquens TK 0001-Zelle comprising at least one exogenous, codon-optimised nucleic acid sequence encoding a glyoxylate reductase from the bacterium Escherichia coli K-12 MG1655, in particular cells of the strain Methylorubrum extorquens Mea-GA1 deposited on 10 Jun. 2022 at the DSMZ, German Collection of Microorganisms and Cell Cultures, Braunschweig, Germany under the deposit number DSM 34286.

In a preferred embodiment of the present invention, the present invention relates to a genetically modified Methylorubrum extorquens TK 0001 cell comprising an exogenous, codon-optimised nucleic acid sequence encoding a glyoxylate reductase from the bacterium Escherichia coli K-12 MG1655 and an exogenous nucleic acid sequence encoding an ethylmalonyl-CoA mutase from the bacterium Methylorubrum extorquens TK 0001 DSM 1337 sequence, in particular cells of the strain Methylorubrum extorquens Mea-GA2 deposited on 10 Jun. 2022 at the DSMZ, German Collection of Microorganisms and Cell Cultures, Braunschweig, Germany under the deposit number DSM 34287.

In a preferred embodiment of the present invention, the present invention relates to a genetically modified Methylorubrum extorquens TK 0001 cell comprising an exogenous, codon-optimised nucleic acid sequence encoding a glyoxylate reductase from the bacterium Escherichia coli K-12 MG1655 and an exogenous nucleic acid sequence encoding an ethylmalonyl-CoA mutase from the bacterium Rhodobacter sphaeroides ATCC 17029, in particular cells of the strain Mea-GA3, deposited on 10 Jun. 2022 at the DSMZ, German Collection of Microorganisms and Cell Cultures, Braunschweig, Germany under the deposit number DSM 34288.

In a preferred embodiment of the present invention, the genetically modified Methylobacteriaceae cell is a cell of the strain Methylorubrum rhodesianum Mrh-GA4 (DSM 34697), Methylorubrum rhodesianum Mrh-GA5 (DSM 34698), Methylorubrum zatmanii Mza-GA14 (DSM 34701), Methylorubrum extorquens Mea-GA17 (DSM 34702), Methylobacterium radiotolerans Mra-GA12 (DSM 34700) or Methylobacterium organophilum Mor-GA8 (DSM 34699), each deposited on 19 Jul. 2023 at the DSMZ, German Collection of Microorganisms and Cell Cultures, Braunschweig, Germany. All deposits were made in accordance with the Budapest Treaty on the International Recognition of the Deposit of Microorganisms for the Purposes of Patent Procedure.

In a preferred embodiment of the present invention, the present invention relates to a genetically modified Methylobacteriaceae cell comprising an exogenous, codon-optimised nucleic acid sequence (SEQ ID NO: 3) encoding a glyoxylate reductase from the bacterium Escherichia coli K-12 MG1655, in particular cells of the strain Methylorubrum zatmanii Mza-GA14 (M. zatmanii DSM 5688+pTE1887-ghrA_{eco}) deposited on 19 Jul. 2023 at the DSMZ, German Collection of Microorganisms and Cell Cultures, Braunschweig, Germany under the deposit number DSM 34701.

In a preferred embodiment of the present invention, the present invention relates to a genetically modified Methylobacteriaceae cell comprising an exogenous, codon-optimised nucleic acid sequence (SEQ ID NO: 3) encoding a glyoxylate reductase from the bacterium Escherichia coli K-12 MG1655 of the strain Methylorubrum extorquens Mea-GA17 (M. extorquens PA1 DSM 23939+pTE1887-ghrA_{eco}) deposited on 19 Jul. 2023 at the DSMZ, German Collection of Microorganisms and Cell Cultures, Braunschweig, Germany under the deposit number DSM 34702.

In a preferred embodiment of the present invention, the present invention relates to a genetically modified Methylobacteriaceae cell comprising an exogenous, codon-optimised nucleic acid sequence (SEQ ID NO: 3) encoding a glyoxylate reductase from the bacterium Escherichia coli K-12 MG1655 of the strain Methylorubrum rhodesianum Mrh-GA4 (M. rhodesianum DSM 5687+pTE1887-ghrA_{eco}) deposited on 19 Jul. 2023 at the DSMZ, German Collection of Microorganisms and Cell Cultures, Braunschweig, Germany under the deposit number DSM 34697.

In a preferred embodiment of the present invention, the present invention relates to a genetically modified Methylobacteriaceae cell comprising an exogenous, codon-optimised nucleic acid sequence encoding a glyoxylate reductase from the bacterium Escherichia coli K-12 MG1655 (SEQ ID NO: 3) and an exogenous native nucleic acid sequence (SEQ ID NO: 4) encoding an ethylmalonyl-CoA mutase from the bacterium Methylorubrum extorquens TK 0001 DSM 1337, of the strain Methylorubrum rhodesianum Mrh-GA5 (M. rhodesianum DSM 5687+pTE1887-ghrA_{eco}-ecmₘₑₐ) deposited on 19 Jul. 2023 at the DSMZ, German Collection of Microorganisms and Cell Cultures, Braunschweig, Germany under the deposit number DSM 34698.

In a preferred embodiment of the present invention, the present invention relates to a genetically modified Methylobacteriaceae cell comprising an exogenous, codon-optimised nucleic acid sequence (SEQ ID NO: 3) encoding a glyoxylate reductase from the bacterium Escherichia coli K-12 MG1655 of the strain Methylobacterium organophilum Mor-GA8 (M. organophilum DSM 18172+pTE1887-ghrA_{eco}-ecmₘₑₐ) deposited on 19 Jul. 2023 at the DSMZ, German Collection of Microorganisms and Cell Cultures, Braunschweig, Germany under the deposit number DSM 34699.

In a preferred embodiment of the present invention, the present invention relates to a genetically modified Methylobacteriaceae cell comprising an exogenous, codon-optimised nucleic acid sequence (SEQ ID NO: 3) encoding a glyoxylate reductase from the bacterium Escherichia coli K-12 MG1655 and an exogenous native nucleic acid sequence (SEQ ID NO: 4) encoding an ethylmalonyl-CoA mutase from the bacterium Methylorubrum extorquens TK 0001 DSM 1337, of the strain Methylobacterium radiotolerans Mra-GA12 (M. radiotolerans DSM 760+pTE1887-ghrA_{eco}-ecmₘₑₐ) deposited on 19 Jul. 2023 at the DSMZ, German Collection of Microorganisms and Cell Cultures, Braunschweig, Germany under the deposit number DSM 34700.

In a particularly preferred embodiment of the present invention, it relates to the specifically deposited Methylobacteriaceae cells, in particular the specifically deposited Methylorubrum strains, as well as each derivative thereof.

In a preferred embodiment of the present invention, the at least one exogenous nucleic acid sequence encoding a glyoxylate reductase from the bacterium Escherichia is functionally connected to additionally at least one regulatory unit by forming an expression cassette, in particular a promoter, in particular an inducible, derepressible or constitutive promoter, an enhancer, a ribosomal binding site and/or a terminator.

In a preferred embodiment of the present invention, the at least one exogenous nucleic acid sequence encoding an ethylmalonyl-CoA mutase, in particular an ethylmalonyl-CoA mutase from at least one bacterium selected from the group consisting of Methylorubrum extorquens, in particular Methylorubrum extorquens TK 0001 DSM 1337, and Rhodobacter sphaeroides, in particular Rhodobacter sphaeroides ATCC 17029, is functionally connected to additionally at least one regulatory unit by forming an expression cassette, in particular a promoter, in particular an inducible, derepressible or constitutive promoter, an enhancer, a ribosomal binding site and/or a terminator.

In a preferred embodiment, the expression cassette is present in a vector, in particular an expression vector, in particular an episomal expression vector, in particular pTE1887.

In a particularly preferred embodiment, the at least one exogenous nucleic acid sequence encoding a glyoxylate reductase and the at least one exogenous nucleic acid sequence encoding an ethylmalonyl-CoA mutase can be present on the same expression vector or on different expression vectors.

In a preferred embodiment of the present invention, the promoter is an inducible promoter, in particular an IPTG-inducible promoter, in particular the P_{L/O4/A1} promoter.

A further aspect of the present invention is a process for producing a genetically modified Methylobacteriaceae cell according to the invention, comprising the process steps:
a) providing a Methylobacteriaceae cell, in particular a wild-type cell, and an expression vector or a genome editing system comprising at least one exogenous nucleic acid sequence encoding a glyoxylate reductase from the bacterium Escherichia, in particular an expression cassette comprising this nucleic acid sequence,
b) transforming the Methylobacteriaceae cell with the expression vector or the genome editing system under conditions that enable the uptake and, preferably stable, subsequent integration of the at least one exogenous nucleic acid sequence into the Methylobacteriaceae cell, and
c) obtaining the genetically modified Methylobacteriaceae cell having at least one exogenous nucleic acid sequence encoding a glyoxylate reductase from the bacterium Escherichia.

In a particularly preferred embodiment, the present invention relates to a abovementioned process, wherein in process step a) additionally at least one exogenous nucleic acid sequence encoding an ethylmalonyl-CoA mutase, in particular from at least one bacterium selected from the group consisting of Methylorubrum extorquens, in particular Methylorubrum extorquens TK 0001 DSM 1337, and Rhodobacter sphaeroides, in particular Rhodobacter sphaeroides ATCC 17029, in particular an expression cassette or genome editing system comprising this nucleic acid sequence, is provided, in process step b) the Methylobacteriaceae cell is transformed with the exogenous nucleic acid sequence encoding an ethylmalonyl-CoA mutase, in particular the expression cassette comprising them, and in process step c) a genetically modified Methylobacteriaceae cell having at least one exogenous glyoxylate reductase, which additionally has at least one exogenous nucleic acid sequence encoding an ethylmalonyl-CoA mutase is obtained.

In a preferred embodiment of the present invention, the transforming according to process step b) is carried out by means of chemical, physical and/or electrical transformation processes, in particular electroporation.

The present invention also relates to a genetically modified Methylobacteriaceae cell that is producible by means of a process according to the invention, in particular has been produced.

A further aspect of the present invention is a genetically modified Methylobacteriaceae cell according to the invention, wherein the cell is present alive, dead, lyophilised, in the form of a cell lysate or a cell extract, and wherein the cell lysate or the cell extract, in particular protein extract, has been recovered from a genetically modified Methylobacteriaceae cell according to the invention. According to the invention, it is provided that the genetically modified Methylobacteriaceae cell according to the invention, which may be present dead, lyophilised or in the form of a cell lysate or cell extract, has the property provided according to the invention of converting at least one Cx compound in a reaction medium into glycolic acid and optionally lactic acid.

In a preferred embodiment of the present invention, the cell, which is present alive or dead, lyophilised or in the form of a cell lysate or a cell extract, catalyses at least the conversion of at least one Cx compound into glycolic acid, in particular the conversion of a reactant containing at least one Cx compound into a product containing glycolic acid, in particular glycolic acid and lactic acid.

A further aspect of the present invention is a biocatalyst comprising a genetically modified Methylobacteriaceae cell according to the invention or a genetically modified Methylobacteriaceae cell according to the invention present dead, lyophilised or in the form of a cell lysate or a cell extract, wherein it is arranged on a carrier, in particular immobilised.

In a preferred embodiment of the present invention, the carrier is an organic carrier or an inorganic carrier. In a preferred embodiment of the present invention, the carrier comprises a naturally occurring organic carrier, in particular consists of it, in particular wherein the carrier is selected from the group consisting of chitin, agar, agarose, alginate, carrageenan and a combination thereof.

In a preferred embodiment of the present invention, the carrier comprises a synthetic organic carrier, more particularly consists of it, in particular wherein the carrier is selected from the group consisting of polyvinyl alcohol (PVA), polyurethane, acrylamide, polypropylene ammonium and a combination thereof.

In a preferred embodiment of the present invention, the carrier comprises an inorganic carrier, in particular consisting of it, in particular wherein the carrier is selected from the group consisting of activated carbon, zeolite, ceramic, clay, anthracite, porous glass and a combination thereof.

In a preferred embodiment of the present invention, the carrier is a composite mixture of an organic carrier and an inorganic carrier, in particular comprising or consisting of polyvinyl alcohol-sodium alginate (PVA-NA), polyvinyl alcohol guar gum (PVA-GG) or both.

In a preferred embodiment of the present invention, the biocatalyst according to the invention catalyses at least the conversion of at least one Cx compound into glycolic acid, in particular converting a reactant containing at least one Cx compound, in particular consisting thereof, into a product containing glycolic acid, in particular glycolic acid and lactic acid, in particular consisting thereof.

A further aspect of the present invention is a bioreactor comprising a genetically modified Methylobacteriaceae cell according to the invention or a biocatalyst according to the invention, wherein the genetically modified Methylobacteriaceae cell or the biocatalyst according to the invention is present in particular in a reaction medium in the bioreactor.

A further aspect of the present invention is a process for producing glycolic acid from at least one Cx compound, in particular a product containing glycolic acid from a reactant containing at least one Cx compound, wherein x is preferably =1, 2 or 4, comprising the process steps:
x) providing a genetically modified Methylobacteriaceae cell according to the invention or a biocatalyst according to the invention, a reaction medium and at least one Cx compound, in particular a reactant containing at least one Cx compound,
y) converting the at least one Cx compound, in particular the reactant, in the reaction medium under conditions that enable the formation of glycolic acid from the Cx compound, and
z) obtaining glycolic acid, in particular the product containing glycolic acid, from the reaction medium.

In a preferred embodiment of the present invention, the Methylobacteriaceae cell according to the invention provided in process step x) or the biocatalyst according to the invention provided in process step x) is present in the reaction medium in suspended form or immobilised form.

In a preferred embodiment of the present invention, the reaction medium provided in process step x) and/or used in process step y) is an aqueous salt-containing solution, in particular a culture medium, in particular a minimal medium, in particular a minimal medium consisting of, per litre of reaction medium, up to 10 g of a Cx compound, in particular methanol, methane, formic acid, methylamine, acetic acid or succinic acid or a mixture thereof, 1 g ammonium sulphate, 450 mg magnesium sulphate heptahydrate, 3.2 mg calcium chloride dihydrate, 7.4 mg trisodium citrate hydrate, 190 μg zinc sulphate heptahydrate, 110 μg manganese chloride tetrahydrate, 2.75 mg iron sulphate heptahydrate, 1.36 mg ammonium heptamolybdate tetrahydrate, 140 μg copper sulphate pentahydrate, 260 μg cobalt chloride hexahydrate, 390 μg sodium tungstate, 30 μg boric acid, 2.02 g potassium dihydrogen phosphate and 4.14 g disodium hydrogen phosphate dihydrate.

In a preferred embodiment of the present invention, the reaction medium provided in process step x) has the reactant containing at least one Cx compound.

In a preferred embodiment of the present invention, the reaction medium provided in process step x) has at the beginning of process step y) the reactant containing at least one Cx compound in a concentration of 1 to 100 g, in particular 5 to 90 g, in particular 6 to 80 g, in particular 7 to 70 g, in particular 8 to 40 g, in particular 9 to 30 g, in particular 10 to 20 g of Cx compound per litre of reaction medium.

In a preferred embodiment of the present invention, the reaction medium provided in process step x) has coenzyme B12.

In a preferred embodiment of the present invention, the reactant used according to the invention, containing at least one Cx compound, is the only carbon source in the reaction medium. Accordingly, in a preferred embodiment, a reaction medium is used which has, as the only carbon source for the Methylobacteriaceae cells, the reactant used, containing at least one Cx compound.

Preferably, according to the invention, it is provided that the converting in process step y) is carried out with continuous or batchwise addition of glyoxylate.

In a preferred embodiment of the present invention, the Cx compound of the reactant provided in process step x) and converted in process step y) is formic acid, methanol, methane, methylamine, acetic acid, succinic acid or a mixture thereof.

In a preferred embodiment of the present invention, the reactant provided in process step x) and converted in process step y) consists of formic acid, methanol, methane, methylamine, acetic acid, succinic acid or a mixture thereof.

In a preferred embodiment of the present invention, is x=1 in the Cx compound of the reactant containing at least one Cx compound provided in process step x) and converted in process step y).

In a preferred embodiment of the present invention, the Cx compound of the reactant provided in process step x) and converted in process step y) is methanol, formic acid or a mixture thereof.

In a preferred embodiment of the present invention, the reactant provided in process step x) and converted in process step y) consists of methanol, formic acid or a mixture thereof.

In a preferred embodiment of the present invention, the reactant provided in process step x) and converted in process step y) consists of methanol.

In a preferred embodiment of the present invention, the reactant provided in process step x) and converted in process step y) consists of formic acid.

In a preferred embodiment of the present invention, the reactant provided in process step x) and converted in process step y) contains methanol and formic acid, in particular 1 to 99 wt. %, in particular 2 to 98 wt. %, in particular 10 to 90 wt. %, in particular 30 to 70 wt. %, in particular 40 to 60 wt. %, in particular 50 wt. %, methanol and in particular 1 to 99 wt. %, in particular 2 to 98 wt. %, in particular 10 to 90 wt. %, in particular 30 to 70 wt. %, in particular 40 to 60 wt. %, in particular 50 wt. %, formic acid (each based on total weight of the reactant provided in process step x)) or consists of these proportions.

In a preferred embodiment of the present invention, the reactant provided in process step x) containing at least one Cx compound, in particular methanol, formic acid or a mixture thereof, in particular methanol, is present at the beginning of process step y) in an initial concentration of 1 to 20 g L⁻¹, in particular 3 to 17 g L⁻¹, in particular 5 to 15 g L⁻¹, in particular 7 to 13 g L⁻¹, in particular 9 to 11 g L⁻¹, in particular 10 g L⁻¹, in the reaction medium.

In a preferred embodiment of the present invention, the reactant provided in process step x) is methanol and is present at the beginning of process step y) in an initial concentration of 1 to 20 g L⁻¹, in particular 3 to 17 g L⁻¹, in particular 5 to 15 g L⁻¹, in particular 7 to 13 g L⁻¹, in particular 9 to 11 g L⁻¹, in particular 10 g L⁻¹, in the reaction medium.

In a preferred embodiment of the present invention, the Cx compound, in particular C1 compound, in particular methanol, formic acid or a mixture thereof, provided in process step x) and converted in process step y) is produced from CO₂, CO or a mixture in a process step w), in particular a process step w) which is operated with renewable energy, in particular electricity from solar, wind, geothermal or hydroelectric energy.

In a preferred embodiment of the present invention, the Cx compound, in particular methanol, formic acid or mixtures thereof, provided in process step x) and converted in process step y), is produced from CO₂, in particular synthesis gas comprising a mixture of CO₂, CO and H₂, in a process step w), in particular by means of a heterogeneous catalytic chemical process, in particular an electrochemical process.

In a preferred embodiment of the present invention, the Cx compound, in particular acetic acid, provided in process step x) and converted in process step y), is produced from CO₂, in particular synthesis gas comprising a mixture of CO₂, CO and H₂, in a process step w) by means of gas fermentation.

In a preferred embodiment of the present invention, the Cx compound, in particular methanol, provided in process step x) and converted in process step y), is produced from CO₂, in particular synthesis gas comprising a mixture of CO₂, CO and H₂, or CO₂, H₂O and electric current, or CO₂ and H₂, in a process step w) by means of an electrochemical process, biochemical process, bioelectrochemical process or gas fermentation.

In a preferred embodiment of the present invention, the CO₂, in particular synthesis gas, used in process step w) is produced by chemical conversion, in particular thermocatalytic conversion, of organic compounds or materials, in particular of sewage sludge and other biogenic residual and waste materials.

In a preferred embodiment of the present invention, the synthesis gas used in process step w) is produced from sewage sludge.

In a preferred embodiment of the present invention, the CO₂ used in process step w) is recovered from the atmosphere or from industrial waste gases.

In a preferred embodiment, the present invention thus makes it possible to enable a sustainable synthesis of glycolic acid and lactic acid that is cost-effective, environmentally friendly and easy to handle and which is almost completely without, in particular without, the use of fossil resources and/or almost completely without, in particular without, biogenic raw materials. According to the invention, glycolic acid and lactic acid are advantageously recovered in a completely renewable way via an integrated process cascade from CO₂ as the only raw material, thus without the consumption of fossil or biogenic resources. Preferably according to the invention, glycolic acid is advantageously produced in a completely renewable way from CO₂ by the present invention.

In a preferred embodiment of the present invention, the reaction medium in process step y) has a temperature of 20 to 40° C., in particular 22 to 38° C., in particular 24 to 36° C., in particular 28 to 32° C., in particular 30° C.

In a preferred embodiment of the present invention, process step y) is carried out in a water vapour-saturated atmosphere.

In a preferred embodiment of the present invention, the reaction medium has a pH value at the beginning of process step y) of pH 4 to 8, in particular 5 to 7, in particular 6, in particular 6.8.

In a preferred embodiment of the present invention, the reaction medium has a pH value after 40 h of reaction time of process step y) of pH 0 to 6, in particular 0 to 4, in particular 0 to 3, in particular 1 to 2.

In a preferred embodiment of the present invention, converting according to process step y) is carried out under mechanical agitation, in particular shaking or stirring.

In a preferred embodiment of the present invention, in process step y), stirring is carried out at 50 to 1000 rpm, in particular 50 to 500 rpm, in particular 50 to 250 rpm, in particular 100 to 200 rpm, in particular 150 rpm (rpm: revolutions per minute).

In a preferred embodiment of the present invention, the reaction medium obtained in process step z) has the product containing at least glycolic acid.

In a preferred embodiment of the present invention, the product obtained in the reaction medium in process step z) containing glycolic acid, glycolic acid or a product containing glycolic acid and lactic acid.

In a preferred embodiment of the present invention, the product obtained in process step z) contains glycolic acid and lactic acid, in particular 1 to 99 wt. %, in particular 2 to 98 wt. %, in particular 10 to 90 wt. %, in particular 30 to 80 wt. %, in particular 40 to 70 wt. %, in particular 50%, in particular 60 wt. %, glycolic acid and in particular 1 to 99 wt. %, in particular 2 to 98 wt. %, in particular 10 to 90 wt. %, in particular 20 to 70 wt. %, in particular 30 to 60 wt. %, in particular 50%, in particular 40 wt. %, lactic acid (each based on total weight of the product obtained in process step z)) or consists of these proportions.

In a preferred embodiment of the present invention, the product obtained in process step z) consists of glycolic acid.

In a preferred embodiment of the present invention, the product obtained in process step z) consists of glycolic acid and lactic acid.

In a preferred embodiment of the present invention, after process step z), in a process step z1), the product, containing glycolic acid and optionally lactic acid, is isolated from the reaction medium, in particular separated from the reaction medium and the genetically modified Methylobacteriaceae cell according to the invention or the biocatalyst according to the invention, in particular by decantation, salting out with a base, in particular NaOH or KOH, filtration, in particular membrane filtration or column filtration, or ion exchange chromatography in combination with HPLC, extraction and/or distillation.

In a preferred embodiment of the present invention, the process for producing a product containing glycolic acid is a continuous process.

A further aspect of the present invention is a process for producing polyglycolic acid, polylactic acid or polylactide-co-glycolide, comprising carrying out a process according to the invention for producing glycolic acid, in particular a product containing glycolic acid and optionally lactic acid, and subsequently polymerising the glycolic acid, lactic acid or glycolic acid and lactic acid obtained from these processes.

In the context of the present invention, a ‘genetically modified Methylobacteriaceae cell according to the invention’ is understood to mean a genetically modified Methylobacteriaceae cell which preferably resembles, in particular is identical to, the wild-type strain of the Methylobacteriaceae cell, with the exception of the presence of at least one exogenous nucleic acid sequence encoding a glyoxylate reductase from the bacterium Escherichia, which gives the Methylobacteriaceae cell according to the invention the advantageous enzymatic glyoxylate reductase activity according to the invention, and optionally associated therewith exogenous nucleic acid sequences of an expression vector or an expression cassette and optionally at least one exogenous nucleic acid sequence encoding an ethylmalonyl-CoA mutase and, where appropriate, exogenous nucleic acid sequences of an expression vector or an expression cassette associated therewith.

In the context of the present invention, ‘a’ genetically modified Methylobacteriaceae cell is understood to mean one, two, several, many or an uncountable number of Methylobacteriaceae cells. In a preferred embodiment of the present invention, a Methylobacteriaceae cell is also understood to mean a Methylobacteriaceae strain, in particular Methylorubrum extorquens-, in particular Methylorubrum rhodesianum-, in particular Methylorubrum zatmanii-, in particular Methylorubrum extorquens TK 0001-, in particular Methylorubrum extorquens AM1-, in particular Methylorubrum extorquens PA1-, in particular Methylobacterium organophilum-, in particular Methylobacterium radiotolerans strain.

In the context of the present invention, a ‘derivative’ of a deposited Methylobacteriaceae cell or a deposited Methylobacteriaceae strain, in particular a deposited Methylorubrum strain or cell or a deposited Methylobacterium strain or cell, is understood to mean a Methylobacteriaceae cell, in particular a Methylobacteriaceae strain, in particular a Methylorubrum cell, in particular a Methylorubrum strain, or a Methylobacterium cell, in particular a Methylobacterium strain, or a Methylobacterium cell, in particular a Methylobacterium strain, which is distinguished by the presence of the features provided according to the invention, in particular the integration of the exogenous nucleic acid sequence encoding a glyoxylate reductase, and has been recovered from a deposited Methylobacteriaceae cell and whose genome has been altered while retaining the features of the invention.

In the context of the present invention, an ‘exogenous nucleic acid sequence’ of an organism, in particular a microorganism, in particular a bacterium, is understood to mean a nucleic acid sequence introduced into a recipient organism by means of recombinant, thus genetic engineering means, process steps.

In the context of the present invention, in a preferred embodiment, an ‘exogenous nucleic acid sequence’ of an organism, in particular a microorganism, in particular a bacterium, is understood to mean a nucleic acid sequence that originates from a different microorganism strain, in particular of another type of organism, in particular of another type of bacterium, and thus non-endogenous, and thus non-native or not occurring in the wild-type strain or wild-type species, is understood.

In the context of the present invention, a ‘glyoxylate reductase’ is understood to be an enzyme that is able to catalyse the conversion of glyoxylate, in particular into glycolic acid, in particular by using a cofactor, in particular NADH or NADPH.

In the context of the present invention, a ‘glyoxylate reductase’ (ghrA) of the present invention has, in a preferred embodiment, a K_{M} value of at most 2.0, in particular at most 1.5, in particular at most 1.0, in particular at most 0.6 mM, in particular 0.6 mM, for glyoxylate.

In the context of the present invention, a ‘glyoxylate reductase’ (ghrA) of the present invention has, in a preferred embodiment, a K_{M} value of at least 0.9, in particular at least 1.0 mM, in particular 1.0 mM, for hydroxypyruvate.

In the context of the present invention, a ‘glyoxylate reductase’ (ghrA) of the present invention has, in a preferred embodiment, a K_{M} value of at most 2.0, in particular at most 1.5, in particular at most 1.0, in particular at most 0.6 mM, in particular 0.6 mM, for glyoxylate and a K_{M} value of at least 0.9, in particular at least 1.0 mM, in particular 1.0 mM, for hydroxypyruvate.

In the context of the present invention, a ‘glyoxylate reductase’ (ghrA) of the present invention has, in a preferred embodiment, a K_{M} value of at most 2.0 for glyoxylate and a K_{M} value of at least 0.9 for hydroxypyruvate.

Preferably the glyoxylate reductase is NADPH-dependent.

In contrast, a ‘hydroxypyruvate reductase’ (ghrB) has a K_{M} value of at least 3.0, in particular at least 4.0, in particular at least 5.0, in particular at least 6.0 mM, in particular at least 6.6 mM, in particular 6.6 mM for glyoxylate. In particular, a hydroxypyruvate reductase (ghrB) has a K_{M} value of at most 0.6, in particular at most 0.7 mM, in particular 0.7 mM, for hydroxypyruvate.

In particular, a ‘hydroxypyruvate reductase’ (ghrB) has a K_{M} value of at least 3.0, in particular at least 4.0, in particular at least 5.0, in particular at least 6.0 mM, in particular at least 6.6 mM, in particular 6.6 mM for glyoxylate and a K_{M} value of at most 0.6, in particular at most 0.7 mM, in particular 0.7 mM, for hydroxypyruvate.

Preferably the hydroxypyruvate reductase is NADH-dependent.

For the definition of the Michaelis-Menten constant, K_{M}, which is defined as the substrate concentration at which the half-maximum conversion rate of a specific enzyme is achieved under fixed reaction conditions, the Lineweaver-Burk evaluation method is preferred as the calculation method, described in Lineweaver, H. and Burk, D. (1934) Determination of the enzyme dissociation constants. J. Am. Chem. Soc. 56, 658-666.

In the context of the present invention, a ‘glyoxylate reductase’ of the present invention has, in a preferred embodiment, a higher enzyme activity in an NADPH-dependent conversion of glyoxylate into glycolate than in an NADH-dependent conversion of glyoxylate into glycolate, in particular an enzyme activity that is at least three times higher, in particular under conditions as indicated in the enzyme assay according to example 5.

In the context of the present invention, a ‘cell of a methylotrophic bacterium’ is understood to mean, in particular a cell that belongs to the family Methylobacteriaceae. In particular, these cells are able to carry out the serine cycle (https://doi.org/10.1002/9781118960608.gbm02024, https://doi.org/10.1111/1462-2920.12736, https://doi.org/10.3389/fmicb.2021.740610).

The serine cycle is a methylotrophic metabolic pathway that enables the assimilation of C1 substrates such as methanol, formate/formic acid, methylamines in microbial metabolism for the formation of biomass or chemical products/intermediates of this metabolism. It is a defined sequence of enzymatically catalysed reactions.

The cycle starts with glycine. In a first step, the assimilated carbon C1 (thus methanol, formic acid, etc.) is converted into the name giving amino acid L-serine in the form of 5,10-methylenetetrahydrofolate, a molecule of water and glycine by a glycine hydroxymethyltransferase (EC 2.1.2.1). Thereby, tetrahydrofolate is split off and prepared for a new carbon assimilation. The L-serine is deaminated by a transaminase in the serine cycle in subsequent steps to form hydroxypyruvate. The split-off NH3 equivalent is used for a transamination of glyoxylate to glycine to keep the cycle running. The aforementioned hydroxypyruvate is reduced by a hydroxypyruvate reductase with NAD(P)H to glycerate, which is phosphorylated by a kinase to 3-phosphoglycerate. In two successive reaction steps, a conversion of the 3-phosphoglycerate into phosphoenolpyruvate is carried out by a phosphoglyceromutase (EC 5.4.2.11) and a water-splitting enolase (EC 4.2.1.11). The phosphoenolpyruvate is carboxylated to oxaloacetate by phosphoenolpyruvate carboxylase (EC 4.1.1.31) using hydrogen carbonate/dissolved CO2. The phosphoenolpyruvate is finally converted via L-malate to L-malyl-CoA with the expenditure of NADH and ATP as well as a cofactor A (CoA) molecule. Subsequently, acetyl-CoA is split off and glyoxylate is formed. This reaction, which is carried out by a malyl-CoA lyase (EC 4.1.3.24), closes the cycle and a further assimilation of a single carbon can begin. (Anthony, C. W. (2011). ‘How half a century of research was required to understand bacterial growth on C1 and C2 compounds; the story of the serine cycle and the ethylmalonyl-CoA pathway.’ Science progress 94 Pt 2: 109-137).

The serine cycle can be detected by the presence of the metabolite hydroxypyruvate. Besides, the characteristic labelling of glycine, serine and glyoxylate can be measured with 13C-labelled C1 substrate and unlabelled CO₂ in labelling studies (https://doi.org/10.1186/1752-0509-5-189).

In the context of the present invention, ‘M. extorquens’ is understood to mean Methylorubrum extorquens, ‘M. rhodesianum’ is understood to mean Methylorubrum rhodesianum, ‘M. zatmanii’ is understood to mean Methylorubrum zatmanii, ‘M. organophilum’ is understood to mean Methylobacterium organophilum, and ‘M. radiotolerans’ is understood to mean Methylobacterium radiotolerans.

In the context of the present invention, ‘pTE1887’ is understood to mean a specific expression vector.

In the context of the present invention, ‘ghrA_{eco}’ is understood to mean a nucleic acid sequence encoding the glyoxylate reductase from Escherichia coli K-12 MG1655. This nucleic acid sequence may be the native (‘ghrA_{eco}-native’) or a codon-optimised (‘ghrA_{eco}-c-optimised’) nucleic acid sequence.

In the context of the present invention, ‘pTE1887-ghrA_{eco}’ is understood to mean an expression vector, which contains the nucleic acid sequence encoding the glyoxylate reductase from Escherichia coli K-12 MG1655.

In the context of the present invention, ‘ecmₘₑₐ’ is understood to mean the nucleic acid sequence encoding the ethylmalonyl-CoA mutase from M. extorquens TK 0001 DSM 1337. This nucleic acid sequence may be the native or a codon-optimised nucleic acid sequence.

In the context of the present invention, ‘pTE1887-ghrA_{eco}-ecmₘₑₐ’ is understood to mean an expression vector which contains the nucleic acid sequence encoding the glyoxylate reductase from Escherichia coli K-12 MG1655 and the nucleic acid sequence encoding the ethylmalonyl-CoA mutase from M. extorquens TK 0001 DSM 1337.

In the context of the present invention, ‘ecmᵣₛₕ’ is understood to mean the nucleic acid sequence encoding the ethylmalonyl-CoA mutase from Rhodobacter sphaeroides ATCC 17029. This nucleic acid sequence may be the native or a codon-optimised nucleic acid sequence.

In the context of the present invention, ‘pTE1887-ghrA_{eco}-ecmᵣₛₕ’ is understood to mean an expression vector that contains the nucleic acid sequence encoding the glyoxylate reductase from Escherichia coli K-12 MG1655 and the nucleic acid sequence encoding the ethylmalonyl-CoA mutase from Rhodobacter sphaeroides ATCC 17029.

In the context of the present invention, a ‘functional nucleic acid sequence equivalent’ is understood to mean a nucleic acid sequence equivalent of a nucleic acid sequence encoding a glyoxylate reductase or an ethylmalonyl-CoA mutase, respectively, wherein the nucleic acid equivalent has at least one difference at at least one nucleotide position from the nucleic acid sequence, this means, has at least one further nucleotide, thus an inserted nucleotide, or at least one missing nucleotide, thus a deleted nucleotide, or has at least one substituted nucleotide, and wherein the nucleic acid equivalent encodes an amino acid sequence with the enzymatic activity of a glyoxylate reductase or an ethylmalonyl-CoA mutase. In the context of the present invention, ‘codon-optimised’ is understood to mean that the nucleic acid sequence of a gene of a wild type, which is to be integrated as an exogenous nucleic acid sequence into a Methylobacteriaceae host cell, in particular from E. coli, before integration is optimised by a genetically engineered exchange of codons for a expression, thus transcription and translation in the host cell, and in particular of those codons which in the exogenous nucleic acid sequence are usually not or not optimally used by the translation system of the host cell, thus the Methylobacteriaceae cell, in particular Methylorubrum extorquens-, in particular Methylorubrum extorquens AM1, Methylorubrum extorquens TK 0001, in particular Methylorubrum extorquens PA1 cell. For example, by means of in vitro mutagenesis, the corresponding methylobacteriaceae-preferred codons are incorporated instead, without the amino acid sequence encoded by the nucleic acid sequence being changed. In the context of the present invention, a codon-optimised nucleic acid sequence is thus a nucleic acid sequence optimised for expression in a Methylobacteriaceae cell. Codon optimisation may also be carried out if the exogenous nucleic acid sequence originates from the same bacterial species as the host cell, but an improvement in expression is nevertheless sought. Preferably, codon optimisation can be carried out according to the following overview (Table 1): In the context of the present invention, ‘functional nucleic acid sequence equivalent of a codon-optimised nucleic acid’ is also understood to mean, but not sole, the native, naturally occurring nucleic acid.

TABLE 1
Codon optimisation

| | Codon 1 | Codon 2 | Codon 3 | Codon 4 | Codon 5 | Codon 6 | Total |
|---|---|---|---|---|---|---|---|
| Amino acid | (proportion) | (proportion) | (proportion) | (proportion) | (proportion) | (proportion) | (proportion) |
| Methionine | AUG (1.00) | — | — | — | — | — | 1.00 |
| Alanine | GCC (0.55) | GCG (0.39) | GCU (0.03) | GCA (0.03) | — | — | 1.00 |
| Arginine | CGC (0.64) | CGG (0.26) | CGU (0.06) | CGA (0.02) | AGG (0.02) | AGA (0.01) | 1.01 |
| Asparagine | AAC (0.86) | AAU (0.14) | — | — | — | — | 1.00 |
| Aspartate | GAC (0.72) | GAU (0.28) | — | — | — | — | 1.00 |
| Cysteine | UGC (0.96) | UGU (0.04) | — | — | — | — | 1.00 |
| Glutamate | GAG (0.79) | GAA (0.21) | — | — | — | — | 1.00 |
| Glutamine | CAG (0.91) | CAA (0.09) | — | — | — | — | 1.00 |
| Glycine | GGC (0.76) | GGG (0.13) | GGU (0.07) | GGA (0.04) | — | — | 1.00 |
| Histidine | CAC (0.74) | CAU (0.26) | — | — | — | — | 1.00 |
| Isoleucine | AUC (0.95) | AUU (0.04) | AUA (0.00) | — | — | — | 0.99 |
| Leucine | CUC (0.48) | CUG (0.43) | CUU (0.04) | UUG (0.04) | CUA (0.01) | UUA (0.00) | 1.00 |
| Lysine | AAG (0.94) | AAA (0.06) | — | — | — | — | 1.00 |
| Phenylalanine | UUC (0.94) | UUU (0.06) | — | — | — | — | 1.00 |
| Proline | CCG (0.62) | CCC (0.34) | CCU (0.03) | CCA (0.01) | — | — | 1.00 |
| Serine | UCG (0.42) | AGC (0.27) | UCC (0.26) | AGU (0.02) | UCU (0.02) | UCA (0.02) | 1.01 |
| Threonine | ACC (0.64) | ACG (0.33) | ACU (0.02) | ACA (0.02) | — | — | 1.01 |
| Tryptophan | UGG (1.00) | — | — | — | — | — | 1.00 |
| Tyrosine | UAC (0.76) | UAU (0.24) | — | — | — | — | 1.00 |
| Valine | GUC (0.50) | GUG (0.46) | GUU (0.03) | GUA (0.01) | — | — | 1.00 |
| Stop | UGA (0.70) | UAA (0.16) | UAG (0.15) | — | — | — | 1.01 |

In the context of the present invention, a ‘functional amino acid sequence equivalent’ is understood to mean an amino acid sequence equivalent of an amino acid sequence of a glyoxylate reductase or an ethylmalonyl-CoA mutase, wherein the amino acid equivalent has at least one difference at at least one amino acid position from the amino acid sequence, that means has at least one further amino acid, thus an inserted amino acid, or at least one missing amino acid, thus a deleted amino acid, or has at least one substituted amino acid, and wherein the amino acid equivalent has the enzymatic activity of a glyoxylate reductase or an ethylmalonyl-CoA mutase.

In the context of the present invention, the ‘identity of nucleic acid or amino acid sequences’ is understood to mean a degree of identity in % determined by a sequence comparison. This sequence comparison is generally based on the BLAST algorithm established and commonly used in the state of the art (see, for example, Altschul et al. (1990) ‘Basic local alignment search tool’, J. Mol. Biol. 215:403-410, and Altschul et al. (1997): ‘Gapped BLAST and PSI-BLAST: a new generation of protein database search programs’, Nucleic Acids Res. 25:3389-3402) and is done by assigning similar sequences of nucleotides or amino acids in the nucleic acid or amino acid sequences to each other. A tabular assignment of the respective positions is referred to as an alignment. A further algorithm available in the prior art is the FASTA algorithm. Sequence comparisons (alignments), in particular multiple sequence comparisons, are carried out using computer programs. For example, the Clustal series is frequently used (see, for example, Chenna et al. (2003) ‘Multiple sequence alignment with the Clustal series of programs’, Nucleic Acids Res. 31:3497-3500), T-Coffee (see, for example Notredame et al. (2000) ‘T-Coffee: A novel method for multiple sequence alignments’, J. Mol. Biol. 302:205-217) or programmes based on these programmes or algorithms. Furthermore, sequence comparisons (alignments) are possible using the computer programme Vector NTI® Suite 10.3 (Invitrogen Corporation, 1600 Faraday Avenue, Carlsbad, California, USA) with the default standard parameters, whose AlignX module for sequence comparisons is based on ClustalW. Unless otherwise stated, the sequence identity given herein is determined using the NCBI Constraint-based Multiple Alignment tool (COBALT) (https://www.ncbi.nlm.nih.gov/, as of 26 Jan. 2022), wherein SEQ ID NO: 1 to 8 are each used as a reference for determining the percentage sequence differences.

Such a comparison also allows a statement to be made about the similarity of the compared sequences to each other. In the present case, it is given as a percentage of ‘identity’, thus the proportion of identical nucleotides or amino acid residues at the same or corresponding positions in an alignment. Statements on identity can be provided for entire polypeptides or genes or only for individual regions. Identical regions of different nucleic acid or amino acid sequences are therefore defined by matches in the sequences. Such regions often have identical functions. They can be small and comprise only a few nucleotides or amino acids. Unless otherwise stated, statements of identity in the present teaching refer to the entire length of the nucleic acid or amino acid sequence given each.

In the context of the present invention, an ‘amino acid sequence’ is understood to mean a sequence of linearly connected amino acids, in particular a protein, in particular a polypeptide.

In the context of the present invention, a ‘nucleic acid sequence’ is understood to mean a sequence of linearly connected nucleotides, in particular a nucleic acid molecule, in particular a gene, in particular a protein-encoding region of a gene. In a particularly preferred embodiment, the nucleic acid sequence is a DNA sequence.

In the context of the present invention, ‘ethylmalonyl-CoA mutase’ is understood to mean a coenzyme B12-dependent enzyme with intramolecular isomerase activity that is responsible for converting ethylmalonyl-CoA to methylsuccinyl-CoA in the ethylmalonyl-CoA metabolism, which preferably has the EC classification EC 5.4.99.63.

In the context of the present invention, a ‘Cx compound’ is understood to be a chemical compound containing carbon (C), hydrogen (H) and oxygen (O) that contains x carbon atoms, wherein x is preferably a natural number. According to the invention, x=1, 2 or 4, in particular 1. Preferably, the Cx compound has only C, H and O atoms and accordingly no other atoms.

In the context of the present invention, formic acid is also understood to mean formate, acetic acid is also understood to mean acetate and succinic acid is also understood to mean succinate. In the context of the present invention, ‘integration of an exogenous nucleic acid sequence into a Methylobacteriaceae cell’ or ‘presence of an exogenous nucleic acid sequence in a Methylobacteriaceae cell’ is understood to mean that each nucleic acid sequence referred to is present in the genome of the cell, chromosomally or extrachromosomally, preferably chromosomally.

In a preferred embodiment, the exogenous nucleic acid sequence is present stably integrated, wherein a stable integration of a nucleic acid is one that can be detected and is able to be expressed in the microorganism for at least 2, 3, 5, 10, 20 or 50 generations of the microorganism.

In the context of the present invention, ‘maximum growth rate’ (μₘₐₓ) is understood to mean the rate of cell division, thus microbial growth, of the Methylobacteriaceae cell according to the invention in a reaction medium, in particular a liquid culture medium. The calculation of μₘₐₓ is based on the measured values of the optical density of the culture medium at a wavelength of 600 nm, measured in a photometer (OD₆₀₀) over the time course of the process step. The calculation of μₘₐₓ can be carried out with equation 1, taking into account the measured values of OD₆₀₀ in the growth interval of the fastest observed growth.

$${μ}_{max} = ( ln \frac{O {D}_{600} ( {t}_{y} )}{\frac{O {D}_{600} ( {t}_{x} )}{( {t}_{y} - {t}_{x} )}} ) [ \frac{1}{h} ] \qquad ( Equation 1 )$$

Equation 1 with t_{y}−tₓ as the time interval of the growth interval of the fastest observed growth and t_{y}>tₓ. The time interval is typically given in hours (h). In the context of the present invention, the ‘dry-biomass-substrate-yield’ (Y_{X/S}) is understood to mean the mass of microbial dry biomass (biomass completely dried to a constant weight) in the reaction medium, in particular liquid culture medium, given in a unit of weight such as grams (X, g_{X}), which can be formed by the specific microbial strain from one gram of the Cx compound (S, g_{Cx}). The calculation is carried out graphically with linear regression of the changes in the measured values of the dry biomass (ΔX(t) as a function of the mass of the Cx compound (ΔCx(t)) over time in the process step according to equation 2. According to equation 2, Y_{X/S} is thus the slope of the linearly correlated change over time of the dry biomass X as a function of the change in the mass of the Cx compound. The unit of Y_{X/S} is typically given in g_{X} per g_{Cx}.

$${Y}_{X / S} = \frac{Δ X ( t )}{Δ Cx ( t ) )} [ \frac{{g}_{X}}{{g}_{Cx}} ] \qquad ( Equation 2 )$$

In the context of the present invention, ‘product-substrate-yield’ (Y_{P/S}) is understood to mean the mass of product, given in a unit of weight such as grams (P, g_{P}), that can be formed by the specific microbial strain from one gram of the Cx compound (S, g_{Cx}). The calculation is carried out graphically with linear regression of the changes in the measured values of the product mass (ΔP(t) as a function of the mass of the Cx compound (ΔCx(t)) over time in the process step according to equation 3. According to equation 3, Y_{P/S} is thus the slope of the linearly correlated change over time of the product mass P as a function of the change in the mass of the Cx compound. The unit of Y_{P/S} is typically given in g_{P} per g_{Cx}.

$${Y}_{P / S} = \frac{Δ P ( t )}{Δ Cx ( t )} [ \frac{{g}_{P}}{{g}_{Cx}} ] \qquad ( Equation 3 )$$

In the context of the present invention, ‘product-dry-biomass-yield’ (Y_{P/X}) is understood to mean the mass of product, given in a unit of weight such as grams (P, g_{P}), which can be formed by the specific microbial strain per gram of dry biomass (X, g_{X}) during microbial growth. The calculation is carried out graphically with linear regression of the changes in the measured values of the product mass (ΔP(t) as a function of the dry biomass (ΔX(t)) over time in the process step according to equation 4. According to equation 4, Y_{P/X} is thus the slope of the linearly correlated change over time of the product mass P as a function of the change in the dry biomass formed. The unit of Y_{P/X} is typically given in g_{P} per g_{X}.

$${Y}_{P / X} = \frac{Δ P ( t )}{Δ X ( t )} [ \frac{{g}_{P}}{{g}_{X}} ] \qquad ( Equation 4 )$$

In the context of the present invention, ‘dry biomass’ is understood to mean the mass X(t) of microbial dry biomass (biomass completely dried to a constant weight) in the reaction medium, in particular liquid culture medium with the volume v(t) at time t, given in a unit of weight such as grams (X, g_{X}). The dry biomass X can be determined from the measured values of the OD₆₀₀ using the correlation factor z according to equation 5, wherein z=0.305 g_{X} per 1 OD₆₀₀ is defined.

$$X ( t ) = 0 , 305 ⋆ {OD}_{600} ( t ) ⋆ v [ gx ] \qquad ( Equation 5 )$$

In the context of the present invention, the abbreviation ‘NAD’ is understood to mean nicotinamide adenine dinucleotide. In the context of the present invention, ‘NADH’ is understood to mean the reduced form of NAD. In the context of the present invention, the abbreviation ‘NADP’ is understood to mean nicotinamide adenine dinucleotide phosphate. In the context of the present invention, ‘NADPH’ is understood to mean the reduced form of NADP. In the context of the present invention, ‘NADH/NADPH analogue’ is understood to mean a chemical compound such as thionicotinamide adenine dinucleotide (S-NAD), nicotinamide adenine dinucleotide (O-NAD), nicotinamide-hypoxanthine dinucleotide (NHD), nicotinamide-guanine dinucleotide, or further compounds that have a similar, preferably the same activity as NADH and/or NADPH.

In the context of the present invention, a ‘reactant’ is understood to mean a starting material, in particular at least one Cx compound, in particular one Cx compound or two or more or many Cx compounds, in particular a composition of Cx compounds.

In the context of the present invention, a ‘product’ is understood to mean at least glycolic acid, in particular glycolic acid alone, preferably glycolic acid and lactic acid, in particular a composition of compounds containing glycolic acid, in particular consisting of the compounds glycolic acid and lactic acid.

In the context of the present invention, ‘converting’ is understood to mean a chemical reaction, in particular a catalysed chemical reaction, in particular an enzymatically catalysed reaction.

In the context of the present invention, a ‘reaction medium’ is understood to mean a liquid medium, in particular a liquid aqueous medium, in which a conversion, in particular an enzymatically catalysed conversion, can take place, in particular a conversion effected by microorganisms or components of microorganisms, in particular a culture medium, in particular a minimal medium.

In the context of the present invention, the term ‘obtaining a product’ is understood to mean that the product recovered in a preceding process step by converting the reactant, thus a starting material, is made available from the reaction medium, in particular a culture medium or solvent, respectively, and in particular is isolated from this. In particular, obtaining a product is therefore to be understood as concentrating, in particular isolating, the product. The processes used for this purpose may be physical, chemical and/or biological processes.

In the context of the present invention, a ‘compound’ is understood to mean a molecule or several identical molecules.

In the context of the present invention, a composition containing glycolic acid is the product of a conversion according to the invention in process step b).

If, in the context of the present invention, quantitative statements, in particular percentages, are given for components of a product or a composition, these add up, unless explicitly stated otherwise or are apparent for person skilled in the art, together with the other components of the composition or product that are explicitly stated or are apparent for a person skilled in the art to 100% of the composition and/or product.

In the context of the present invention, the term ‘at least one’ is understood to mean a quantity of 1 or 2 or 3 or 4 or 5 or 6 or 7 or 8 or 9 or 10 or more. In a particularly preferred embodiment, the term ‘at least one’ can represent exactly the number 1. In a further preferred embodiment, the term ‘at least one’ can also mean 2 or 3 or 4 or 5 or 6 or 7.

Insofar in the context of the present invention a ‘presence’, a ‘containing’, a ‘having’ or a ‘content’ of a component is explicitly mentioned or implied, this means that the respective component is present, in particular in a measurable amount.

If, in the context of the present invention, a ‘presence’, ‘containing’ or ‘having’ of a component in a quantity of 0 [unit], in particular mg/kg, μg/kg or wt. %, is explicitly mentioned or implied, this means that the respective components are not present in a measurable amount, in particular are not present.

The number of decimal places given corresponds to the precision of the measurement method used in each case.

If, in the context of the present invention, the first and second decimal place or the second decimal place are not given for a number, these are to be set as zero.

In the context of the present invention, the term ‘and/or’ is understood to mean that all members of a group which are connected by the term ‘and/or’ are disclosed both alternatively to each other and cumulatively in any combination. For the term ‘A, B and/or C’, this means that the following disclosure content is to be understood: a) A or B or C, or b) (A and B), or c) (A and C), or d) (B and C), or e) (A and B and C).

In the context of the present invention, the terms ‘comprising’ and ‘having’ are understood to mean that, in addition to the elements explicitly covered by these terms, further elements not explicitly mentioned may also be included. In the context of the present invention, these terms are also understood to mean that only the explicitly mentioned elements are covered and that no further elements are present. In this particular embodiment, the meaning of the terms ‘comprising’ and ‘having’ is equivalent to the term ‘consisting of’. In addition, the terms ‘comprising’ and ‘having’ also include compositions that, in addition to the explicitly mentioned elements, also contain further not mentioned elements, which, however, are of a functionally and qualitatively subordinate nature.

In this embodiment, the terms ‘comprising’ and ‘having’ are equivalent to the term ‘essentially consisting of’.

The designation ‘DSMZ, German Collection of Microorganisms and Cell Cultures, Braunschweig, Germany’ stands for ‘Leibniz-Institut DSMZ-German Collection of Microorganisms and Cell Cultures GmbH, Inhoffenstraße 7B, 38124 Braunschweig’.

Further preferred embodiments of the present invention are set forth in the subclaims.

The invention is described in more detail below, without limitation of the general inventive concept, by way of examples and associated figures.

The sequence listing shows:

SEQ ID NO: 1 represents the native nucleic acid sequence encoding a glyoxylate reductase from the bacterium Escherichia coli (K-12 MG1655), in particular also referred to as, ghrA_{eco}-native, thus a functional nucleic acid sequence equivalent of the nucleic acid sequence according to SEQ ID NO: 3.

SEQ ID NO: 2 is the amino acid sequence encoded by SEQ ID Nos. 1 and 3.

SEQ ID NO: 3 represents a Methylobacteriaceae-codon-optimised nucleic acid sequence (ghrA_{eco}-c-optimised) of the native nucleic acid sequence according to SEQ ID NO: 1 encoding a glyoxylate reductase from the bacterium Escherichia coli (K-12 MG1655).

SEQ ID NO: 4 represents the native nucleic acid sequence encoding an ethylmalonyl-CoA mutase from the bacterium Methylorubrum extorquens (TK 0001 DSM 1 337), in particular also referred to as ecmₘₑₐ, thus a functional nucleic acid sequence equivalent of the codon-optimised nucleic acid sequence according to SEQ ID NO: 13.

SEQ ID NO: 5 is the amino acid sequence encoded by SEQ ID NO: 4 and 13.

SEQ ID NO: 6 represents the native nucleic acid sequence encoding an ethylmalonyl-CoA mutase from the bacterium Rhodobacter sphaeroides (ATCC 17029), in particular also referred to as ecmᵣₛₕ, thus a functional nucleic acid sequence equivalent of the codon-optimised nucleic acid sequence according to SEQ ID NO: 8.

SEQ ID NO: 7 is the amino acid sequence encoded by SEQ ID Nos. 6 and 8.

SEQ ID NO: 8 represents a Methylobacteriaceae-codon-optimised nucleic acid sequence of the native nucleic acid sequence according to SEQ ID NO: 6 encoding an ethylmalonyl-CoA mutase from the bacterium Rhodobacter sphaeroides (ATCC 17029).

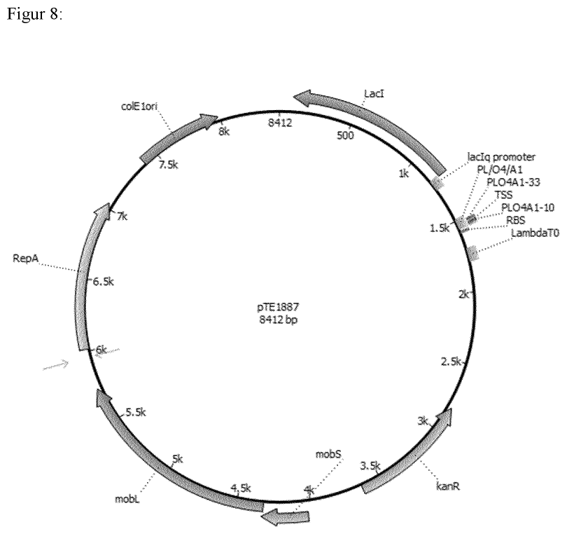

SEQ ID NO: 9 represents the nucleic acid sequence of the expression vector pTE1887, wherein the associated plasmid map is shown in FIG. 8.

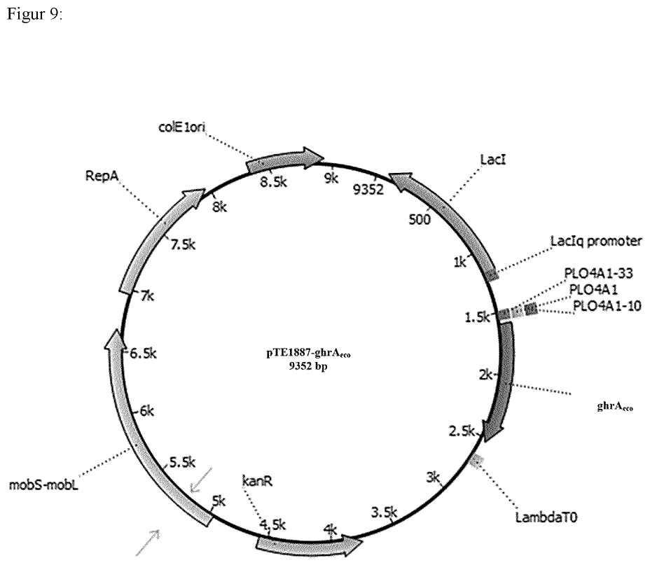

SEQ ID NO: 10 represents the nucleic acid sequence of the expression vector pTE1887-ghrA_{eco}, wherein the associated plasmid map is shown in FIG. 9.

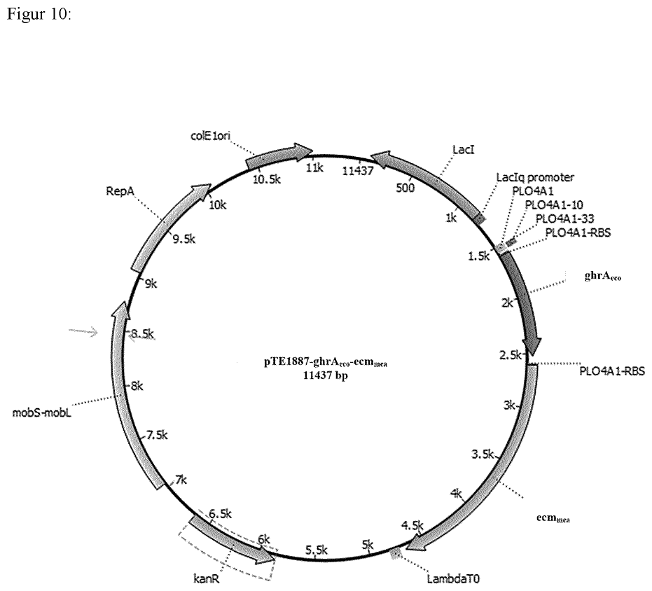

SEQ ID NO: 11 represents the nucleic acid sequence of the expression vector pTE1887-ghrA_{eco}. ecmₘₑₐ, wherein the associated plasmid map is shown in FIG. 10.

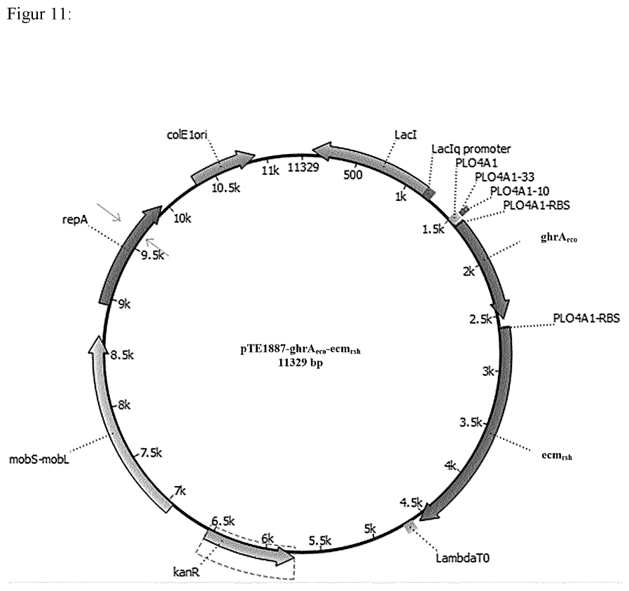

SEQ ID NO: 12 represents the nucleic acid sequence of the expression vector pTE1887-EcoGoxRed_1-ecmᵣₛₕ, wherein the associated plasmid map is shown in FIG. 11.

SEQ ID NO: 13 represents a Methylobacteriaceae codon-optimised nucleic acid sequence of the native nucleic acid sequence according to SEQ ID NO: 4 encoding an ethylmalonyl-CoA mutase from the bacterium Methylorubrum extorquens (TK 0001 DSM 1337).

The figures show:

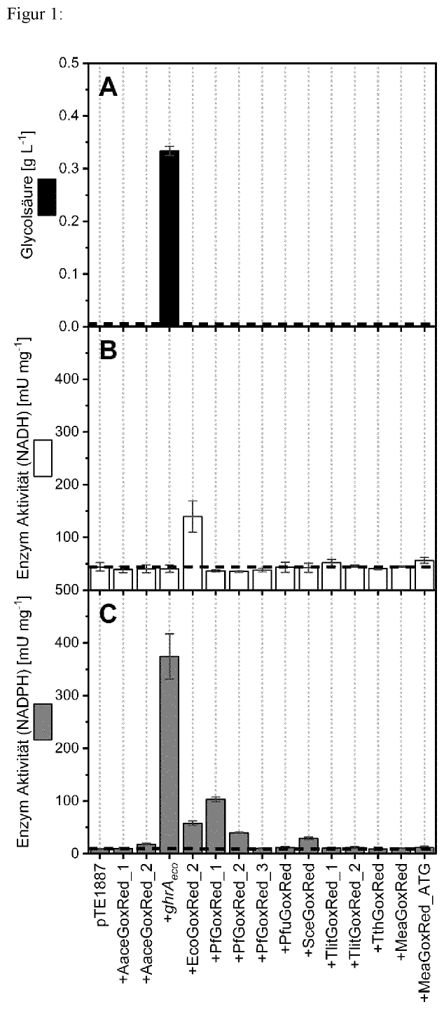

FIG. 1: the screening result for glycolic acid production in recombinant, thus genetically modified, M. extorquens TK 0001 strains that have and express codon-optimised genes of the glyoxylate reductases (A), screening results according to 1A in (B and C), wherein enzyme activities of the glyoxylate reductases from the biomass used according to 1(A) are expressed with the expression vector pTE1887 in the strain background M. extorquens TK 0001 with NADH (B) and NADPH (C) as cofactor are represented,

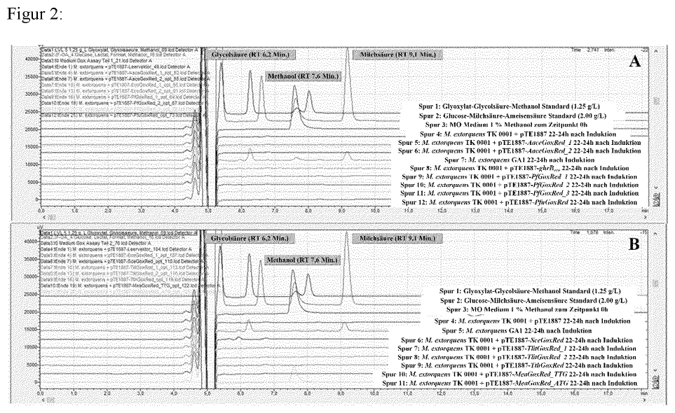

FIG. 2: an HPLC-chromatogram comparison of the cultivation samples (22 to 24 h after induction) of the genetically modified Methylobacteriaceae cells M. extorquens TK 0001 glyoxylate reductase strains, which have and express codon-optimised genes of the glyoxylate reductases,

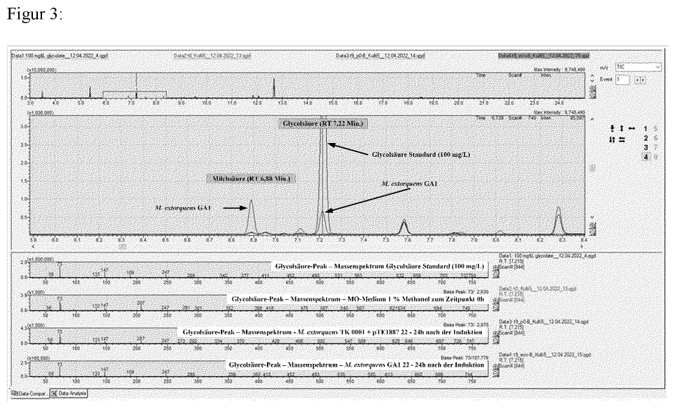

FIG. 3: a GC-MS chromatogram and mass spectra of the glycolic acid peak (retention time: 7.22 min) of a 100 mg L⁻¹ glycolic acid standard, a sample of the reaction medium at t=0 h, a sample of M. extorquens TK 0001+pTE1887 empty vector cultivation after induction, and a sample according to the invention of +pTE1887-ghrA_{eco}-c-optimised (M. extorquens GA1) cultivation after induction,

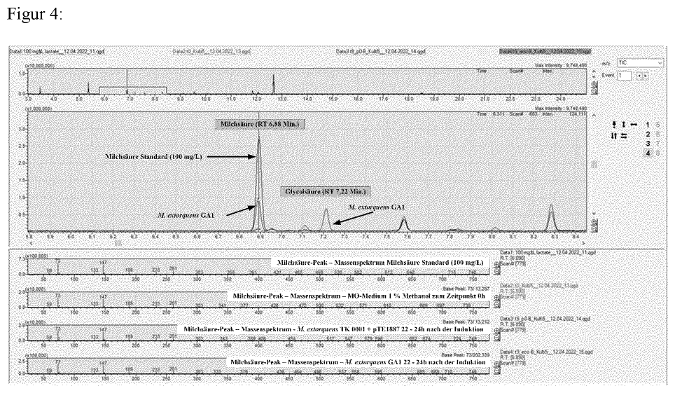

FIG. 4: a GC-MS chromatogram and mass spectra of the lactic acid peak (retention time: 6.88 min) of a 100 mg L⁻¹ lactic acid standard, a sample of the reaction medium at t=0 h, a sample of M. extorquens TK 0001+pTE1887 empty vector cultivation 22-24 h after induction, and a sample of M. extorquens TK 0001+pTE1887-ghrA_{eco}-c-optimised (M. extorquens GA1) cultivation 22-24 h after induction,

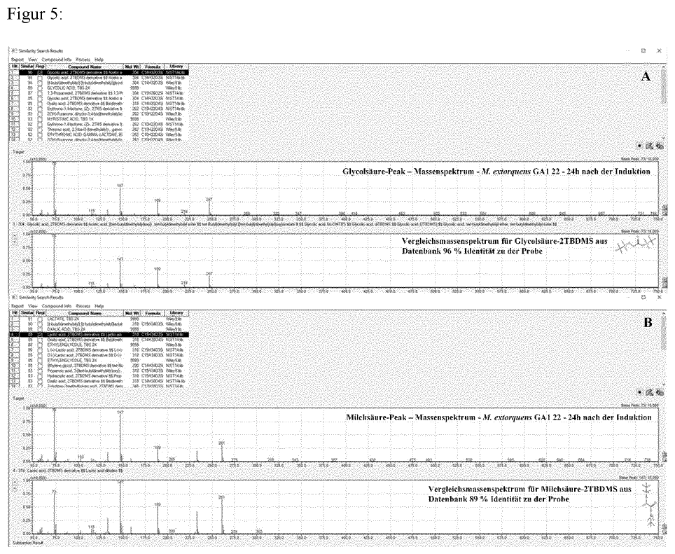

FIG. 5: a detailed view of the mass spectra of the glycolic acid peak (A) and the lactic acid peak (B) of a sample of M. extorquens GA1 cultivation 22 to 24 h after induction and database verification of the glycolic acid identity (A) and lactic acid identity (B) in the M. extorquens GA1 sample,

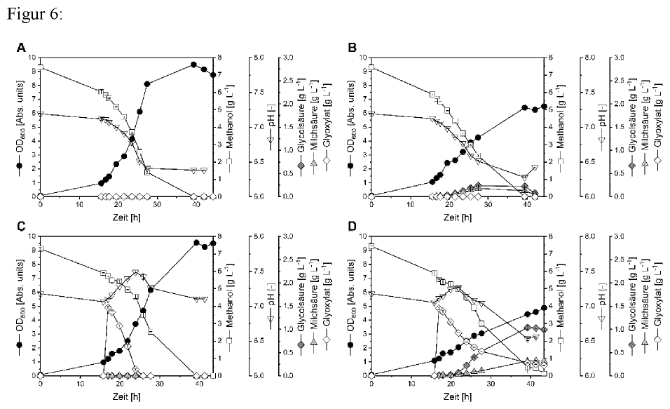

FIG. 6: the growth curve (OD600), the pH value and the methanol, glyoxylate, glycolic acid and lactic acid concentrations of M. extorquens TK 0001+pTE1887 (A+C) and M. extorquens TK 0001+pTE1887-ghrA_{eco}-c-optimised (M. extorquens GA1) according to the invention (B+D) in the reaction medium, namely minimal medium, wherein as carbon source 8 g L⁻¹ methanol (A+B) or 9 g L⁻¹ methanol+1.5 g L⁻¹ glyoxylic acid (C+D) was added,

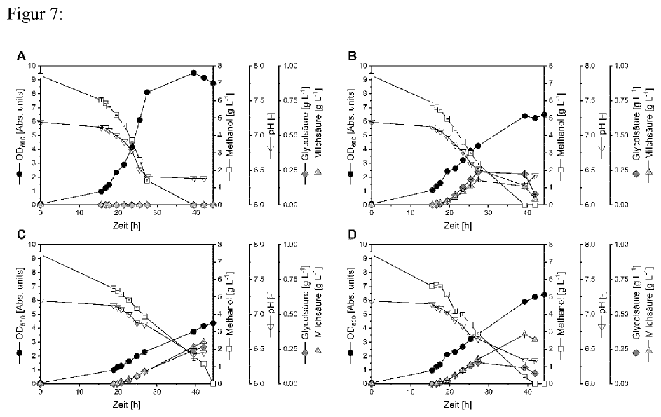

FIG. 7: the growth (OD600), pH value, methanol and the glycolic and lactic acid concentrations of M. extorquens TK 0001+pTE1887 (A), M. extorquens TK 0001+pTE1887-ghrA_{eco}-c-optimised (M. extorquens GA1) according to the invention (B), M. extorquens TK 0001+pTE1887-ghrA_{eco}-c-optimised-ecmₘₑₐ (M. extorquens GA 2) according to the invention (C) and M. extorquens TK 0001+pTE1887-ghrA_{eco}-c-optimised-ecmᵣₛₕ (M. extorquens GA3) according to the invention (D) in a reaction medium with 9 g L⁻¹ methanol as sole substrate,

FIG. 8: the plasmid map of the expression vector pTE1887,

FIG. 9: the plasmid map of the expression vector pTE1887-ghrA_{eco}-c-optimised,

FIG. 10: the plasmid map of the expression vector pTE1887-ghrA_{eco}-c-optimised-ecmₘₑₐ,

FIG. 11: the plasmid map of the expression vector pTE1887-ghrA_{eco}-c-optimised-ecmᵣₛₕ,

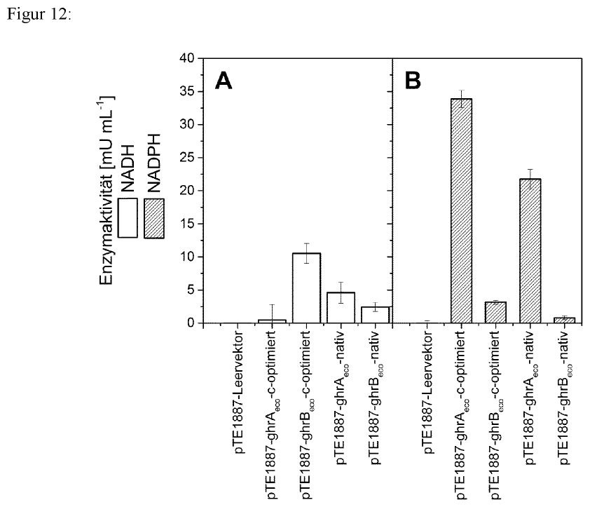

FIG. 12: the results of the glyoxylate reductase enzyme activity tests of ghrA_{eco} and ghrB_{eco} in native and codon-optimised DNA sequence expressed with the expression vector pTE1887 in the strain background M. extorquens TK 0001,

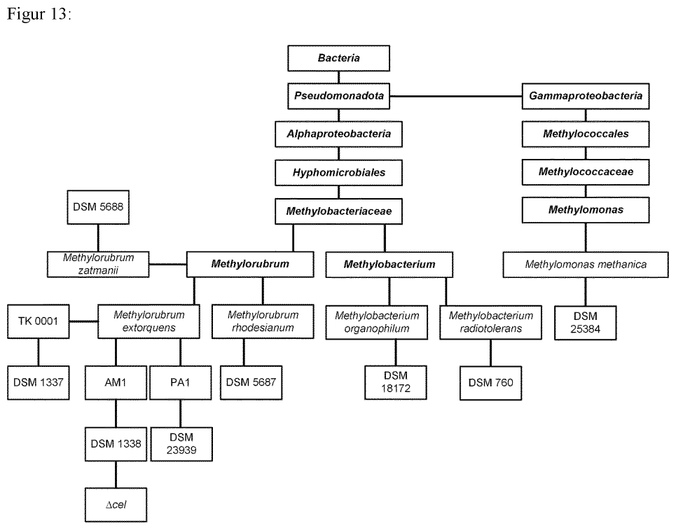

FIG. 13: the taxonomic classification of the methylotrophic microorganisms tested with the expression vectors according to the invention,

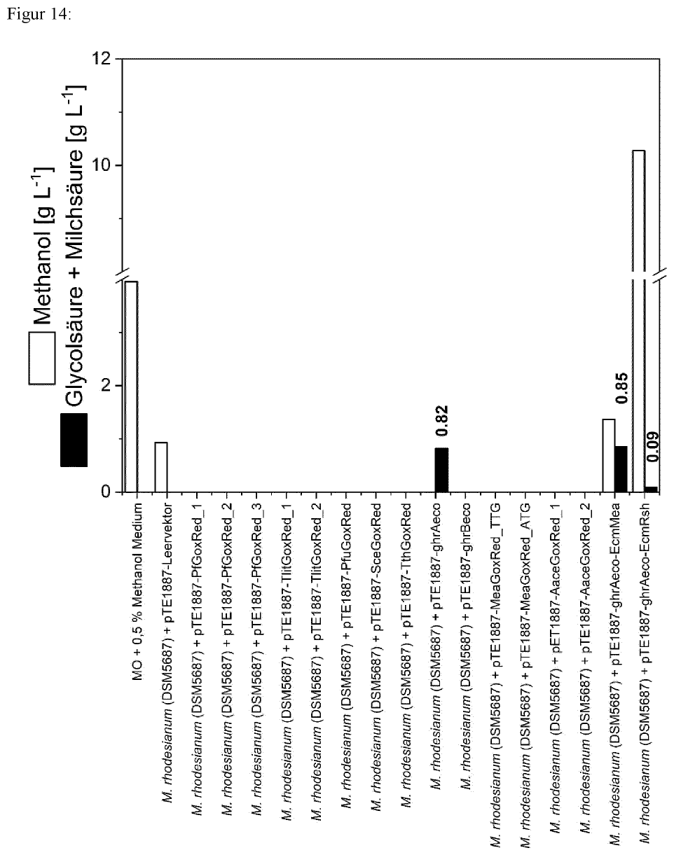

FIG. 14: the screening result for glycolic acid and lactic acid production 22 h to 28 h after induction of gene expression in recombinant, thus genetically modified, M. rhodesianum DSM 5687 strains that have and express codon-optimised genes of the glyoxylate reductases and, in some strains, additionally ethylmalonyl-CoA mutases,

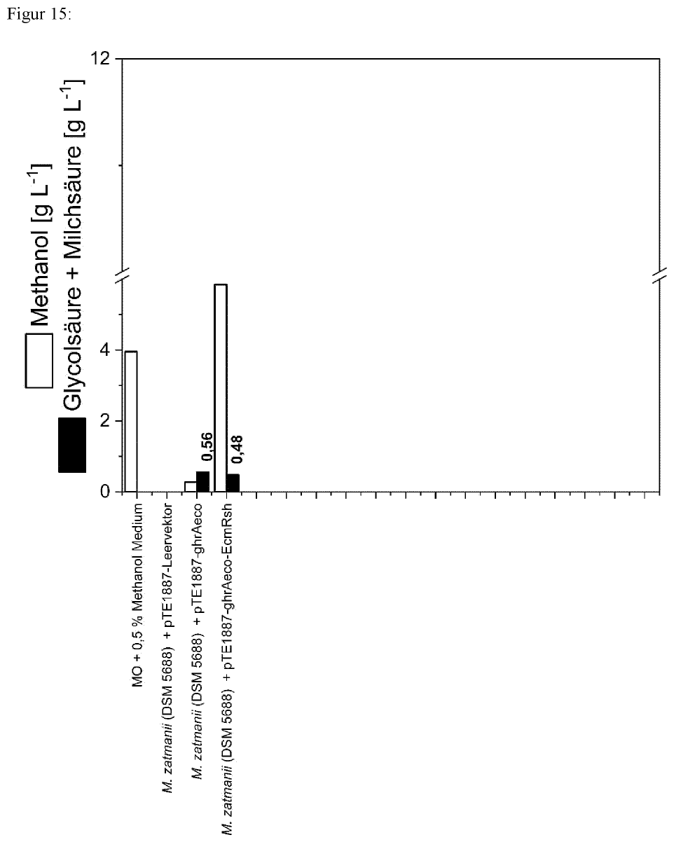

FIG. 15: the screening result for glycolic acid and lactic acid production 22 h to 28 h after induction of gene expression in recombinant, thus genetically modified, M. zatmanii DSM 5688 strains that have and express the codon-optimised gene of glyoxylate reductase from Escherichia according to the invention and in one strain additionally a codon-optimised gene of ethylmalonyl-CoA mutase from Rhodobacter sphaeroides ATCC 17029,

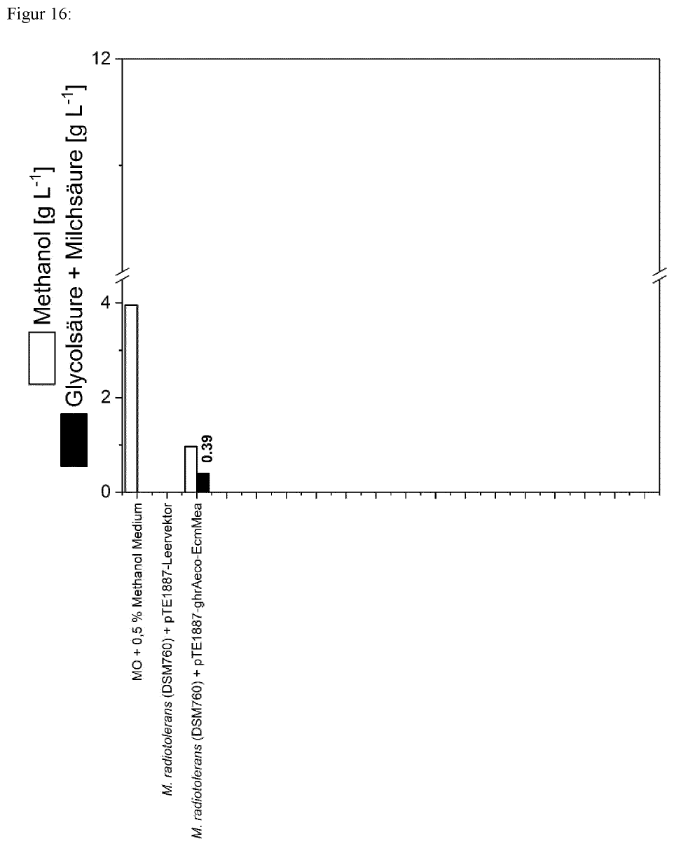

FIG. 16: the screening result for glycolic acid and lactic acid production 22 h to 28 h after induction of gene expression in a recombinant, thus genetically modified, M. radiotolerans DSM 760 strain that has and expresses the combination according to the invention of the codon-optimised gene of glyoxylate reductase from Escherichia and additionally a native gene of the ethylmalonyl-CoA mutase from M. extorquens TK 0001 DSM 1337, and

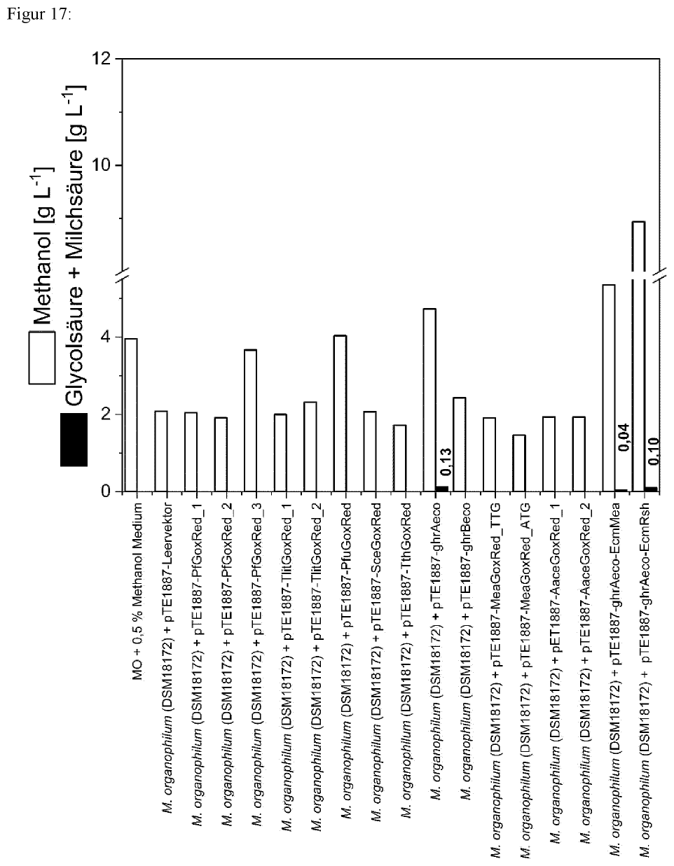

FIG. 17: the screening result for glycolic acid and lactic acid production 22 h to 28 h after induction of gene expression in recombinant, thus genetically modified, M. organophilum DSM 18172 strains that have and express codon-optimised genes of the glyoxylate reductases and in some strains additionally ethylmalonyl-CoA mutases,

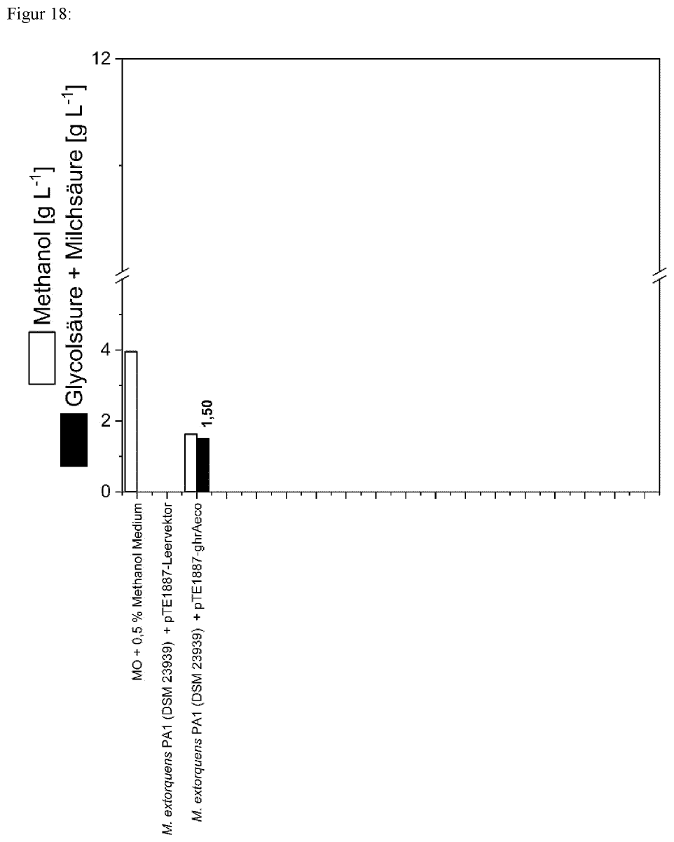

FIG. 18: the screening result for glycolic acid and lactic acid production 22 h to 28 h after induction of the gene expression of a recombinant, thus genetically modified, M. extorquens PA1 DSM 23939 strain, that has and expresses according to the invention the codon-optimised gene of the glyoxylate reductase from Escherichia coli K12 1655,

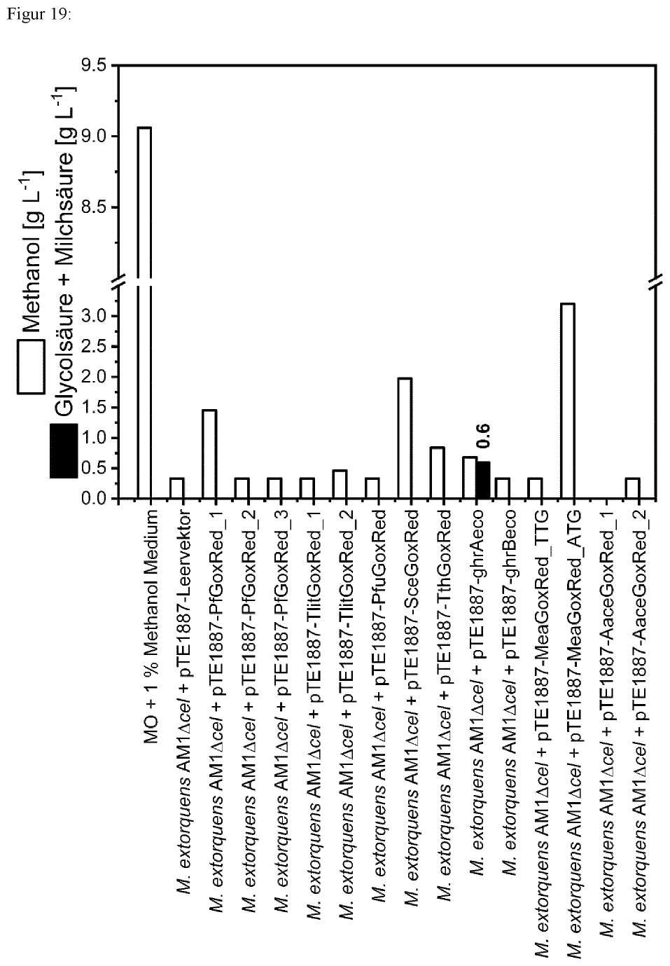

FIG. 19: the screening result for glycolic acid and lactic acid production 22 h to 28 h after induction of gene expression in recombinant, thus genetically modified, M. extorquens AM1Δcel (based on the strain DSM 1338) strains that have and express codon-optimised genes of the glyoxylate reductases.

### EXAMPLES

### Example 1: Production of Genetically Modified Methylobacteriaceae Cells

12 different exogenous glyoxylate reductases were identified and the associated DNA and amino acid sequences were extracted using bioinformatics methods using the KEGG database (www.genome.jp/kegg/) and the Brenda Enzymes database (https://www.brenda-enzymes.org/). Only glyoxylate reductases that occur in prokaryotes or Saccharomyces cerevisiae were considered. The native glyoxylate reductase from M. extorquens TK 0001 was also selected. An overview of the 13 selected glyoxylate reductases is summarised in Table 2. In particular, the glyoxylate reductase from Thermococcus litoralis was identified as an NADH-dependent enzyme (Ohshima, et al., European Journal of Biochemistry, 2001, 268(17): p. 4740-4747). The influence of the specific redox equivalent on glycolic acid production can be substantial, depending on the availability of the specific redox equivalent in the cytosol and the adaptation of the metabolic network to the intervention (overexpression of glyoxylate reductase).

TABLE 2
Summary of the tested enzymes

| | | | | Length of | |
|---|---|---|---|---|---|
| | | | | DNA | Length of amino |
| | | KEGG- | | sequence | acid sequence |
| Name | Enzyme | entry | Origin | [base pairs] | [amino acids] |
| PfGoxRed_1 | 2-Ketogluconate | Pfl01_0936 | Pseudomonas | 981 | 326 |
| | reductase | | fluorescens Pf0-1 | | |
| PfGoxRed_2 | Putative D-isomer specific | Pf101_2771 | Pseudomonas | 966 | 321 |
| | 2-hydroxyacid | | fluorescens Pf0-1 | | |
| | dehydrogenase family | | | | |
| | protein | | | | |
| PfGoxRed_3 | Putative 2-hydroxyacid | Pf101_3899 | Pseudomonas | 930 | 309 |
| | dehydrogenase | | fluorescens Pf0-1 | | |
| TlitGoxRed_1 | Glyoxylate reductase | OCC_02245 | Thermococcus litoralis | 996 | 331 |
| TlitGoxRed_2 | 2-Hydroxyacid dehydrogenase | OCC_08355 | Thermococcus litoralis | 1002 | 333 |
| PfuGoxRed | Putative | PF0319 | Pyrococcus furiosus | 1011 | 336 |
| | phosphoglycerate | | DSM 3638 | | |
| | dehydrogenase | | | | |
| SceGoxRed | Glyoxylate reductase | YNL274C | Saccharomyces cerevisiae | 1053 | 350 |
| TthGoxRed | Glycerate dehydrogenase/ | TT_C0431 | Thermus | 1017 | 338 |
| | glyoxylate reductase | | thermophilus HB27 | | |
| ghrA_{eco} | Glyoxylate/reductase | b1033 | Escherichia coli K- | 939 | 312 |
| (invention) | | | 12 MG1655 | | |
| ghrB_{eco} | Hydro-xypyruvate | b3553 | Escherichia coli K- | 975 | 324 |
| | reductase | | 12 MG1655 | | |
| MeaGoxRed | Putative 2-hydroxyacid | — | Methylorubrum | 996 | 331 |
| | dehydrogenase | | extorquens TK 0001 | | |
| AaceGoxRed_1 | Glyoxylate reductase | AOU92_03200 | Acetobacter aceti | 987 | 328 |
| | (NADP(+)) | | | | |
| AaceGoxRed_2 | Glyoxylate/hydroxypyruvate | AOU92_11415 | Acetobacter aceti | 942 | 313 |
| | reductase A | | | | |

The heterologous enzymes from Pseudomonas fluorescens Pf0-1, Thermococcus litoralis, Pyrococcus furiosus DSM 3638, Saccharomyces cerevisiae, Thermus thermophilus HB27, Escherichia coli K-12 MG1655 and Acetobacter aceti were encoded by synthetic genes in a codon-optimised form for Methylobacteriaceae (BioCat GmbH, Heidelberg, Germany, Table 1) to support the best possible gene expression. Since the homologous gene from M. extorquens (SEQ ID NO: 1) had the start codon ‘TTG’, the start codon was changed to ‘ATG’ by PCR (Kozak, M., Gene, 1999, 234(2): p. 187-208). In further steps, both gene variants of SEQ ID NO: 1 and 3 were tested. This results in 14 variants of the tested glyoxylate reductases.

The synthetic genes were cloned in a codon-optimised form using Gibson assembly on the episomal expression vector pTE1887 (Carrillo, M. et al., ACS Synthetic Biology, 2019, 8(11): p. 2451-2456) under the control of the P_{L/O4/A1} promoter (IPTG-inducible) (FIG. 8). FIG. 8 shows the vector with the following elements: lacI gene, lacI promoter, PL/O4/A1 promoter,→−33 region→−10 region→transcription start, PL/O4/A1 promoter ribosomal binding site (RBS), lambda T0 terminator, kanamycin resistance, mobilisation genes mobS and mobL, regulatory protein RepA, origin of replication colE1.

For this cloning, the expression vector was cut with the restriction enzyme NcoI. The sequence identity and correctness of the constructs were ensured by sequencing. Subsequently, the produced constructs and a wild-type strain Methylobacteriaceae cell, in particular M. extorquens TK 0001 cells and in particular M. extorquens PA1, were according to process step a) provided and according to process step b) transformed into each of the Methylobacteriaceae cells by means of electroporation and a genetically modified Methylobacteriaceae cell is obtained according to process step c). Clones of the Methylobacteriaceae cells, thus genetically modified Methylobacteriaceae cells, which carry the individually produced constructs containing the synthetic genes in codon-optimised form, were selected on minimal medium agar plates with kanamycin as a selection marker. The presence of the expression vectors and the respective expected sequence size of the PCR product which represents the cloned gene were verified by colony PCR in the individual clones obtained. The verified strains were stored at −80° C. as cryocultures.

To test the ability of the genetically modified Methylobacteriaceae strains to produce glycolic acid, the strains, a minimal medium as a reaction medium, in particular also referred to as a culture medium, and a Cx compound with x=1, namely methanol (Cui, L.-Y. et al., Biochemical Engineering Journal, 2017, 119: p. 67-73) were provided (process step x) according to the invention), cultivated in baffled shake flasks (250 mL flask volume, 50 mL culture volume) at 30° C., 150 RPM (revolutions per minute) and in a water vapour-saturated atmosphere (process step y) according to the invention) (New Brunswick™ Innova 44, Eppendorf AG, Hamburg, Germany) and a product containing glycolic acid is obtained in the reaction medium (process step z)). Similarly, cultivation with formic acid, among other things, is also possible which can likewise be used as a reactant for glycolic acid production.

The inoculation of the main cultures was carried out from precultures grown under the same conditions (final OD₆₀₀ between 3 and 5) to a starting OD₆₀₀ of 0.05. After the cultures had reached an OD₆₀₀ of 1.0, the gene expression of the codon-optimised glyoxylate reductase genes was induced with 1 mM IPTG (final concentration in the culture volume). In order to verify the production of glycolic acid, a sample volume of 1 mL of the minimal medium was withdrawn from the culture volume before inoculation and a sample volume of 1 mL respectively from all cultures was withdrawn before induction, directly after induction and about 20 hours after induction. After the biomass was separated from the reaction medium by centrifugation, the samples were analysed using high-performance liquid chromatography (HPLC) and refractive index detection (RID) with regard to the contained concentrations of methanol, formic acid, glyoxylate, glycolic acid and lactic acid. The HPLC measurement was carried out using a Synergi™ 4 μm Hydro-RP 80A, LC column 250×4.6 mm (Phenomenex Inc., Torrance, CA, USA) and 20 mM K₂HPO₄ (pH 1.5) as the eluent at 30° C. and a flow rate of 0.5 mL min⁻¹ for 20 minutes per sample. The identification and quantification of the analytes were carried out using external standards of known concentration.

Glycolic acid was clearly detected in the culture samples by gas chromatography coupled with mass spectrometry (GC-MS) using a glycolic acid standard (100 mg L⁻¹). For this purpose, the —OH and —NH groups contained in the culture samples and standard were converted into the corresponding tert-butyldimethylsilyl ethers (TBDMS) by derivatisation. For this purpose, a volume of 50 μL of standard or 50 μL of sample was lyophilised and subsequently resuspended in 50 μL DMF+0.1% (v/v) pyridine. Derivatisation was carried out with 50 μL N-methyl-N-tert-butyldimethylsilyltrifluoracetamide (MBDSTFA, Macherey-Nagel) and incubation at 80° C. for 30 minutes. Any precipitates that formed were removed by centrifugation and the samples were subsequently analysed using GC-MS. The GC method was set with a carrier gas flow of 1.7 mL min⁻¹, an inlet temperature of 250° C., an interface temperature of 230° C. and a quadrupole temperature of 150° C. The separation of the analytes was carried out using a temperature gradient: 120° C. (2 min), ramp 8° C. min⁻¹ to 200° C. and 10° C. min⁻¹ to 325° C. The analytes were identified in scan mode (m/z 50 to 750) using MS.

Genetically modified Methylobacteriaceae cells comprising an exogenous, codon-optimised nucleic acid sequence (SEQ ID NO: 3) encoding a glyoxylate reductase from the bacterium Escherichia coli K-12 MG1655 of the strain Methylorubrum extorquens Mea-GA1 were deposited on 10 Jun. 2022 at the DSMZ, German Collection of Microorganisms and Cell Cultures, Braunschweig, Germany under the deposit number DSM 34286.

### Example 2: Screening of Functional Glyoxylate Reductases in M. extorquens TK 0001

The glyoxylate reductase-encoding nucleic acid sequences in codon-optimised form listed in Table 2 were cloned into the pTE1887 expression vector as described in example 1 and the corresponding genetically modified Methylobacteriaceae strains were constructed. M. extorquens TK 0001 strain, which contains the pTE1887 vector, was used as a reference strain, which does not carry a recombinant plasmid, but the pTE1887 empty vector.

In a first experimental step, these initially constructed strains, as described in example 1, were examined for their ability to produce glycolic acid. The results are summarised in FIG. 1.

FIG. 1A shows a bar chart, wherein the genetically modified Methylobacteriaceae cells are represented on the x-axis and the y-axis shows the concentration of glycolic acid (black filled bar) in g L⁻¹ in the reaction medium. All sample collection times are 22-24 hours after induction of gene expression with 1 mM IPTG. To determine the amount of methanol consumed, the reaction medium was measured at time t=0 h. All concentrations are given in g L⁻¹, determined by HPLC, refractive index detection and external standards.

FIG. 1A shows the screening result of glycolic acid production in recombinant M. extorquens TK 0001 strains expressing glyoxylate reductases based on the corresponding codon-optimised genes. pTE1887 was used as expression vector, which also serves as a negative control in the form of the empty vector in the reference strain M. extorquens TK 0001+pTE1887 (first entry from the left on the x-axis).

Surprisingly, neither the reference strain M. extorquens TK 0001+pTE1887 (first entry from the left) nor the genetically modified Methylobacteriaceae cells showed any glycolic acid production (entries from the left: 2 and 3 and 5 to 15), with the exception of the genetically modified Methylobacteriaceae cell according to the invention, comprising M. extorquens TK 0001+pTE1887-ghrA_{eco} (in codon-optimised nucleic acid form according to SEQ ID NO: 3), thus a genetically modified Methylobacteriaceae cell according to the invention comprising at least one exogenous nucleic acid sequence encoding a glyoxylate reductase from the bacterium Escherichia (entry from the left: 4, is the only entry to show a black bar). FIG. 9 shows the map of the vector used to generate these Methylorubrum cells with the following elements: lacI gene, lacI promoter, PL/O4/A1 promoter,→−33 region→−10 region→transcription start, PL/O4/A1 promoter ribosomal binding site (RBS), ghrA_{eco}-c-optimised, Lambda T0 terminator, kanamycin resistance, mobilisation genes mobS and mob, regulatory protein RepA. Origin of replication colE1.

FIG. 1B shows a bar chart, wherein the genetically modified Methylobacteriaceae cells are represented on the x-axis and the y-axis represents the enzyme activity in mU mg⁻¹ (white, unfilled bar: NADH as cofactor).

FIG. 1C shows a bar chart wherein the genetically modified Methylobacteriaceae cells are represented on the x-axis and the y-axis represents the enzyme activity in mU mg⁻¹ (grey bar: NADPH as cofactor).

FIGS. 1B and 1C show the screening result of an enzyme assay with recombinant M. extorquens TK 0001 strains that express glyoxylate reductases, based on the corresponding codon-optimised genes. The enzyme assay was carried out in the same way as in example 5. The biomass used was that used in 1A. In the case of 1B, the enzyme assay was carried out with NADH as redox cofactor. In the case of 1C, the enzyme assay was carried out with NADPH as redox cofactor.

In the case of 1B, all tested Methylobacteriaceae cells containing recombinant glyoxylate reductases show no measurable glyoxylate reductase enzyme activity with NADH as cofactor, with the exception of the Methylobacteriaceae cell containing the glyoxylate reductase ghrB_{eco} not according to the invention (entry from the left: 5).

In FIG. 1C, the reference strain M. extorquens TK 0001+pTE1887 (first entry from the left), as well as several tested Methylobacteriaceae cells containing recombinant glyoxylate reductases (entries from the left: 2, 8 and 9, 11 to 15) showed no measurable glyoxylate reductase enzyme activity with NADPH as cofactor. Only the genetically modified Methylobacteriaceae cells (entries from the left: 3 to 7 and 10) showed increased glyoxylate reductase enzyme activity, wherein the genetically modified Methylobacteriaceae cells of M. extorquens TK 0001+pTE1887-ghrA_{eco} (in codon-optimised nucleic acid form according to SEQ ID NO: 3), thus a genetically modified Methylobacteriaceae cell comprising at least one exogenous nucleic acid sequence encoding a glyoxylate reductase from the bacterium Escherichia, had a high glyoxylate reductase enzyme activity (entry from the left: 4).

No enzyme activity of the glyoxylate reductase Tlit could be measured and neither was it associated with a glycolic acid production. Only the enzyme activity of ghrA_{eco}, thus the glyoxylate reductase from E. coli according to the invention, is connected with a glycolic acid production.

Accordingly, the genetically modified Methylobacteriaceae cell comprising M. extorquens TK 0001+pTE1887-ghrA_{eco}-c-optimised according to the invention has NADPH dependence but not NADH dependence.

FIG. 2 shows HPLC chromatograms of the cultivation samples according to FIG. 1 (22 to 24 hours after induction) of the genetically modified Methylobacteriaceae cells M. extorquens TK 0001 glyoxylate reductase strains. It can be clearly seen that only the genetically modified Methylobacteriaceae cell M. extorquens TK 0001+pTE1887-ghrA_{eco} according to the invention (referred to in FIG. 2 as M. extorquens GA1, containing the codon-optimised form of the ghrA_{eco} gene) is the only strain to produce the mixture of glycolic acid and lactic (glycolic acid retention time=6.20 min and lactic acid retention time=9.1 min) (comparison of standards—lane 1 and lane 2—with M. extorquens TK 0001+pTE1887-ghrA_{eco}, M. extorquens GA1—lane 7 in 1A and lane 5 in 1B). The lack of glycolic acid (and lactic acid production when using glyoxylate reductases not according to the invention) indicates a lack of functionality. These enzymes could be hydroxypyruvate reductases, which reduce the hydroxypyruvate that also accumulates in the serine cycle, depending on NAD(P)H, to D-glycerate. In this case, as shown in FIG. 1 and FIG. 2, no accumulation of glycolic acid would be observed.

This sample was analysed by GC-MS in comparison with external standards to confirm the presence of glycolic acid and lactic acid in the sample and thus verify the production of glycolic acid and the surprising production of lactic acid by expression of the ghrA_{eco} enzyme. Further peaks: glyoxylate (retention time=5.40 min), methanol (retention time=7.6 min), peaks not described in further detail here (retention time=6.50 min and 8.00 min).

In the case of the genetically modified Methylobacteriaceae cell according to the invention, about 0.6 g L⁻¹ of the mixture of glycolic acid and lactic acid could be detected in the HPLC measurement (Y_{P/S}˜77 mg_{glycolic acid+lactic acid g methanol}⁻¹).

In most cultivations, the initial methanol concentration of 8 g L⁻¹ was depleted after approximately 22 to 24 h after induction. Only the strain M. extorquens GA1 showed a clearly measurable methanol concentration at the time of sample collection (FIG. 1A, lane 7 and FIG. 1B, lane 5). This may indicate an imbalance in the metabolism caused by the gene expression of the glyoxylate reductase. On the one hand, the enzyme expression itself can reduce the growth of the strains. But increased enzyme activity of a glyoxylate reductase can also cause a reduction of glyoxylate from the serine cycle towards glycolic acid. As a result, the microorganism lacks glyoxylate to build biomass. This deficiency can slow growth and lead to that the carbon source is not completely used up.

To confirm the presence of glycolic acid and lactic acid in the ghrA_{eco} sample according to the invention in comparison to external standards (each 100 mg L⁻¹ lactic acid and glycolic acid), this sample was examined as described in example 1, with a GC-MS measurement (FIG. 3 to FIG. 5).

FIG. 3 shows a GC-MS chromatogram and mass spectra of a 100 mg L⁻¹ glycolic acid standard, a sample of the medium at t=0 h, a sample of M. extorquens TK 0001+pTE1887 empty vector cultivation 22-24 h after induction and a sample according to the invention of M. extorquens TK 0001+pTE1887-ghrA_{eco} (referred to in FIG. 3 as M. extorquens GA1, containing the codon-optimised form of the ghrA_{eco} gene) cultivated 22 to 24 h after induction. FIG. 4 shows the same samples, except for the standard, which was replaced by a 100 mg L⁻¹ lactic acid standard. The measurements verify that glycolic acid was clearly formed in the cultivation sample of M. extorquens TK 0001+pTE1887-ghrA_{eco} (M. extorquens GA1) according to the invention compared to the glycolic acid standard (retention time=7.22 min). The mass spectrum of the obtained peak in the M. extorquens TK 0001+pTE1887-ghrA_{eco} sample according to the invention clearly matches the mass spectrum of the glycolic acid standard (FIG. 5A). This proves the existence of glycolic acid in the M. extorquens TK 0001+pTE1887-ghrA_{eco} sample according to the invention and thus the production of glycolic acid by this strain. In comparison, no glycolic acid can be detected in the sample of the M. extorquens TK 0001+pTE1887 empty vector strain. Surprisingly, the formation of lactic acid (retention time=6.88 min) was also detected exclusively in the sample of the M. extorquens TK 0001+pTE1887-ghrA_{eco} cultivation according to the invention. Here, too, the mass spectrum matches that of the lactic acid standard (FIG. 5B).

This approach clearly showed that glycolic acid was produced by the genetically modified Methylorubrum cell M. extorquens TK 0001+pTE1887-ghrA_{eco} according to the invention. Surprisingly, it was also shown that this strain produces a mixture of glycolic acid and lactic acid (see FIG. 5). FIG. 5 shows detailed mass spectra of an identical sample of the M. extorquens TK 0001+pTE1887-ghrA_{eco} according to the invention (referred to in FIG. 5 as M. extorquens GA1, containing the codon-optimised form of the ghrA_{eco} gene) Cultivation 22 to 24 h after induction with database evidence of the glycolic acid identity in the M. extorquens GA1 sample (A) and the lactic acid identity in the M. extorquens GA1 sample (B).

Surprisingly, in the cultivation of M. extorquens TK 0001+pTE1887-ghrA_{eco} it could be verified by GC-MS that both glycolic acid (retention time=7.22 min) and lactic acid (retention time=6.88 min) were produced (FIGS. 3 to 5). The peak with a retention time of 6.88 min in this sample was identified as lactic acid 2×TBDMS (derivative of lactic acid with MBDSTFA) with an 89-91% probability by comparison with an external standard and by database matching of the mass spectrum (FIG. 5B).

The control strain M. extorquens TK 0001+pTE1887 did not show this phenotype: neither glycolic acid nor lactic acid could be detected as products by GC-MS.

Without wanting to be bound by theory, the changes in the redox balance alter the metabolism of the genetically modified Methylobacteriaceae cell M. extorquens TK 0001+pTE1887-ghrA_{eco} such that lactic acid is synthesised as a possible by-product of glycolic acid production. An NADH-dependent lactate dehydrogenase (KEGG database: Mex_1p4794), which uses pyruvate as a substrate, could be responsible for this lactic acid formation. An alternative possibility is that the glyoxylate reductase has a non-specific substrate utilisation that enables the enzyme to use pyruvate as an acceptor. The course of the methylglyoxal metabolic pathway is also conceivable.

It can therefore be shown that the M. extorquens TK 0001+pTE1887-ghrA_{eco} cells according to the invention containing the codon-optimised form of the ghrA_{eco} gene, produce a mixture of glycolic acid and lactic acid, which can serve as a starting point for polymerisation to polyglycolic acid, polylactic acid or polylactide-co-glycolide.

### Example 3: Growth Experiments

Furthermore, growth experiments were carried out with M. extorquens TK 0001+pTE1887 and, according to the invention, with the strain M. extorquens TK 0001+pTE1887-ghrA_{eco} (M. extorquens GA1) in minimal medium (reaction medium) with 10 g L⁻¹ methanol as one reactant and a mixture of 10 g L⁻¹ methanol+1.5 g L⁻¹ glyoxylate as a further reactant (FIG. 6).

FIGS. 6 A to D show diagrams in which the y-axes represent the growth curve (OD₆₀₀, circles, black filled), the pH value (triangles, tip down) and the methanol (squares, unfilled), glyoxylate (diamonds, unfilled) and glycolic acid concentrations (diamonds, dark grey filled) as well as lactic acid (triangles, grey filled, tip up) of M. extorquens TK 0001+pTE1887 (A+C) and M. extorquens TK 0001+pTE1887-ghrA_{eco} according to the invention (codon-optimised) (B+D) in the minimal medium and the time is given on the x-axis. As carbon source (reactant), thus Cx compound, 10 g L⁻¹ methanol (A+B) or 10 g L⁻¹ methanol+1.5 g L⁻¹ glyoxylate (C+D) was added. The measurement of the methanol, glyoxylate and glycolic acid concentrations was carried out by HPLC, refractive index detection and external standards. All concentrations are given in g L⁻¹. The data represent three independent biological replicates.

The glyoxylate was added at the time of induction of gene expression and serves as a test to see if an in vivo increase in glyoxylate supply leads to an increase in glycolic acid production.

In FIG. 6A it can be recognized that the reference strain M. extorquens TK 0001+pTE1887 with 10 g L⁻¹ methanol as reactant did not produce any glycolic acid and had a uniform biomass formation up to a maximum OD₆₀₀ of approx. 9 after 40 h of cultivation. The significant decrease in the pH value to below 6.5 over the course of the fermentation is striking. In comparison, in a cultivation with M. extorquens TK 0001+pTE1887, the addition of glyoxylate led to slightly delayed growth and a slightly higher maximum OD₆₀₀ of approx. 10 after about 42 h. In this case, too, no glycolic acid was produced (FIG. 6C). However, the pH value in this cultivation could be maintained at the initial pH value of around 7.0, which is probably due to the glyoxylate feeding.

It was shown that the recombinant strain M. extorquens TK 0001+pTE1887-ghrA_{eco} containing the codon-optimised form of the ghrA_{eco} according to the invention, from 10 g L⁻¹ methanol, the products glycolic acid and lactic acid were formed in increased concentrations (˜0.35 g L⁻¹ and 0.25 g L⁻¹ respectively in 40 h). After the methanol has been degraded, the products are completely degraded in the further course of cultivation. The formation of glycolic acid and lactic acid is accompanied by a significant slowdown in biomass growth to a maximum OD₆₀₀ of 6.7 in 44 h. Furthermore, the pH value of the culture broth presumably drops in this case due to the additionally formed glycolic acid to 6.2 and rises due to the degradation of the glycolic acid to a value comparable to that of the reference strain, just under 6.5 (FIG. 6B).

In the experiment with the M. extorquens TK 0001+pTE1887-ghrA_{eco} containing the codon-optimised form of the ghrA_{eco} gene according to the invention, and with the additional feeding of glyoxylate, a significant increase in glycolic acid production of up to 1.0 g L⁻¹ in 44 h was achieved. The amount of lactic acid formed was comparable to that obtained in cultivation without glyoxylate supplementation (6B). This showed that glyoxylate plays an important role as a precursor in the formation of glycolic acid, and that increasing the in vivo concentration of glyoxylate results in improved glycolic acid production. In this experiment, too, the formed glycolic acid and lactic acid were metabolised after the methanol was consumed. The increased product formation in this experiment again led to a further reduction in biomass growth, wherein a maximum OD₆₀₀ of around 4.5 was achieved. In contrast to the reference strain, a significant decrease in pH value can be observed in this case despite the addition of glyoxylate, as glycolic acid and lactic acid were produced. However, an increase in pH value can also be observed during the degradation of the formed glycolic acid and lactic acid after the methanol has been consumed (FIG. 6D).

In summary, the strain-specific cultivation parameters derived from the data, such as (specific growth rate), Y_{X/S} (dry-biomass-substrate-yield), q_{S} (specific substrate uptake rate), Yₚ is (product-substrate-yield) and q_{P} (specific product formation rate) were summarized in Table 3 for the strains M. extorquens TK 0001+pTE1887 and the M. extorquens TK 0001+pTE1887-ghrA_{eco} containing the codon-optimised form of the ghrA_{eco} gene according to the invention. These data suggest that glycolic acid and lactic acid production is associated with a significant reduction in the biomass-substrate-yield (70% of the reference strain and 70% of the reference strain with glyoxylate feeding) and more carbon is converted into the product or has to be used to maintain the redox balance. It can also be seen that glyoxylate reduces the growth rate, indicating a potential toxic effect of the precursor. This toxicity of glyoxylate can be avoided by an optimal balancing of the in vivo glyoxylate pool.

TABLE 3
Summary of cultivation parameters of M. extorquens TK 0001 + pTE1887
and M. extorquens TK 0001 + pTE1887-ghr A_{eco} (M. extorquens GA1) containing
the codon-optimised form of the ghrA_{eco} gene according to the invention, in a
minimal medium with 10 g L⁻¹ methanol or additionally + 1.5 g L⁻¹ glyoxylate.

| | | Y_{X/S} | q_{S} | Y_{P/S} | q_{P} |
|---|---|---|---|---|---|
| | μ | [g_{DBM} | [g_{MeOH} | [g_{GA} | [g_{GA} |
| strain and condition | [1 h⁻¹] | g_{MeOH}⁻¹] | g_{DBM}⁻¹ h⁻¹] | g_{MeOH}⁻¹] | g_{DBM}⁻¹ h⁻¹] |
| M. extorquens TK 0001 + pTE1887 | 0.17 | 0.37 | 0.46 | 0.00 | 0.00 |
| M. extorquens TK 0001 + pTE1887- | 0.10 | 0.26 | 0.38 | 0.07 | 0.06 |
| ghrA_{eco} (according to the invention) | | | | | |
| M. extorquens TK 0001 + pTE1887 + | 0.16 | 0.23 | 0.70 | 0.00 | 0.00 |
| 2 g L⁻¹ glyoxylate¹ | | | | | |
| M. extorquens TK 0001 + pTE1887- | 0.09 | 0.16 | 0.56 | 0.17 | 0.22 |
| ghrA_{eco} + 2 g L⁻¹ glyoxylate¹ | | | | | |
| (according to the invention) | | | | | |

¹Yields are estimated from the cumulative substrate utilisation of methanol and glyoxylate.
Abbreviations: μ, specific growth rate MeOH, methanol; GA, glycolic acid; DBM, dry biomass.

Examples 1 to 3 show that according to the invention glycolic acid and lactic acid can be produced by M. extorquens GA1 from Cx compounds in a methylotrophic fermentation process according to the invention.

It should in particular be emphasised that glycolic acid production according to the invention in M. extorquens GA1 can be significantly increased by increasing the intracellular concentration of glyoxylate, as shown in Example 3. In this case, 185% more glycolic acid was produced compared to cultivation without glyoxylate feeding.

### Example 4: Experimental Data from the Fermentative Production of Glycolic Acid and Lactic Acid from Methanol

The experimental proceedings were carried out according to example 1. The strain used is the wild-type strain Methylorubrum extorquens TK 0001 DSM 1337.

The following expression vectors (1 to 4) were used:
1.) pTE1887 (expression vector, also named as empty vector; plasmid map: FIG. 8)
2) pTE1887-ghrA_{eco} (expression vector which codon-optimised encodes the glyoxylate reductase from Escherichia coli K-12 MG1655; with SEQ ID NO: 3, plasmid map: FIG. 9) (according to the invention)
3) pTE1887-ghrA_{eco}-ecmₘₑₐ (expression vector which encodes the glyoxylate reductase from Escherichia coli K-12 MG1655 (codon-optimised) and the ethylmalonyl-CoA mutase from M. extorquens TK 0001 DSM 1337 natively; plasmid map: FIG. 10) (according to the invention)
4) pTE1887-ghrA_{eco}-ecmᵣₛₕ (expression vector encoding the glyoxylate reductase from Escherichia coli K-12 MG1655 (codon-optimised) and the ethylmalonyl-CoA mutase from Rhodobacter sphaeroides ATCC 17029 codon-optimised; plasmid map: FIG. 11) (according to the invention).

Genetically modified Methylobacteriaceae cells were produced by means of the processes described in example 1.

FIG. 10 shows the map of the vector used to generate the Methylobacteriaceae cells expressing ghrA_{eco}-ecmₘₑₐ, with the following elements: lacI gene, lacI promoter, PL/O4/A1 promoter→−33 region→−10 region→transcription start, PL/O4/A1 promoter ribosomal binding site (RBS), ghrA_{eco} (codon-optimised), ecmₘₑₐ (native), lambda T0 terminator, kanamycin resistance, mobilisation genes mobS and mob, regulatory protein RepA. Origin of replication colE1.

FIG. 11 shows the map of the vector used to generate these Methylobacteriaceae cells, expressing ghrA_{eco}-ecmᵣₛₕ, with the following elements: lacI gene, lacI promoter, PL/O4/A1 promoter→−33 region→−10 region→transcription start, PL/O4/A1 promoter ribosomal binding site (RBS), ghrA_{eco} (codon-optimised), rsh-ecm (codon-optimised), lambda T0 terminator, kanamycin resistance, mobilisation genes mobS and mob, regulatory protein RepA. Origin of replication colE1.

Genetically modified Methylorubrum extorquens TK 0001-cells comprising an exogenous, codon-optimised nucleic acid (SEQ ID NO: 3) encoding a glyoxylate reductase from the bacterium Escherichia coli K-12 MG1655 and an exogenous, native nucleic acid sequence (SEQ ID NO: 4) encoding an ethylmalonyl-CoA mutase from the bacterium Methylorubrum extorquens TK 0001 DSM 1337, of the strain Methylorubrum extorquens Mea-GA2 were deposited on 10 Jun. 2022 at the DSMZ, German Collection of Microorganisms and Cell Cultures, Braunschweig, Germany under the deposit number DSM 34287.

Genetically modified Methylorubrum extorquens TK 0001-cells comprising an exogenous, codon-optimised nucleic acid sequence (SEQ ID NO: 3) encoding a glyoxylate reductase from the bacterium Escherichia coli K-12 MG1655 and an exogenous, codon-optimised nucleic acid sequence (SEQ ID NO: 8) encoding an ethylmalonyl-CoA mutase from the bacterium Rhodobacter sphaeroides ATCC 17029, of the strain Methylorubrum extorquens Mea-GA3 were deposited on 10 Jun. 2022 at the DSMZ, German Collection of Microorganisms and Cell Cultures, Braunschweig, Germany under the deposit number DSM 34288.

Fermentation experiments were carried out using culture medium as the reaction medium and methanol (reactant) as the sole carbon source.

FIG. 7 shows the time course of the biomass concentration (OD₆₀₀) and the medium pH value over the course of the cultivation. At the same time, culture supernatant samples were measured using high-performance chromatography to show the substrate and product concentrations and their changes over time.

FIG. 7 shows the time in hours on the x-axis and on the y-axes the growth curve (OD₆₀₀, circles, black filled), pH value (triangles, tip down), methanol (squares, unfilled) and glycolic (diamonds, dark grey filled) and lactic acid concentrations (triangles, grey filled, tip up) of M. extorquens TK 0001+pTE1887 (A), M. extorquens TK 0001+pTE1887-ghrA_{eco} (codon-optimised) according to the invention (M. extorquens GA1) (B), M. extorquens TK 0001+pTE1887-ghrA_{eco}-ecmₘₑₐ (ghrA_{eco}: codon-optimised; ecmₘₑₐ: native) according to the invention (M. extorquens GA2) (C) and M. extorquens TK 0001+pTE1887-ghrA_{eco}-ecmᵣₛₕ (both genes codon-optimised) according to the invention (M. extorquens GA3) (D) in a culture medium with 10 g L⁻¹ methanol as the sole reactant, thus as a Cx compound.

It was again shown that the enzyme ghrA_{eco}, allows a production of glycolic acid in the background strain M. extorquens TK 0001. Lactic acid is also produced here. In comparison, the wild-type strain, which contains the empty vector pTE1887, does not show production of glycolic acid or lactic acid. In these experiments, approx. 250 mg L⁻¹ glycolic acid and approx. 200 mg L⁻¹ lactic acid were produced with a product substrate yield of around 50 mg gₘₑₜₕₐₙₒₗ⁻¹ (glycolic acid+lactic acid). It can be observed that the initiated glycolic acid synthesis lowers the dry-biomass-substrate-yield (Y_{X/S}) of the strain M. extorquens TK 0001+pTE1887-ghrA_{eco} according to the invention (M. extorquens GA1) to 70% compared to the empty vector strain. The product yield based on the dry biomass (Y_{P/X}) is 0.27 g g_{dry biomass}⁻¹ (Table 4).

Surprisingly, the additionally implementation of the exogenous ethylmalonyl-CoA mutase leads to a significant improvement in glycolic acid production performance compared to the strain M. extorquens TK 0001+pTE1887-ghrA_{eco} according to the invention. The growth of the M. extorquens TK 0001+pTE1887-ghrA_{eco}-ecmₘₑₐ strain according to the invention, with a measured growth rate (μ) of 0.10 h⁻¹ was delayed compared to the empty vector strain (0.17 h⁻¹). The use of the ecm gene from M. extorquens TK 0001 DSM 1337 leads to an increase of the glycolic acid titer by 18% after 40 h of cultivation compared to the strain according to the invention M. extorquens TK 0001+pTE1887-ghrA_{eco} (0.26 g L⁻¹ versus 0.22 g L⁻¹, Table 4). Likewise, the dry-biomass-substrate-yield is reduced by 49% compared to the empty vector strain and by 27% compared to the strain according to the invention M. extorquens TK 0001+pTE1887-ghrA_{eco}.

A slight change can be observed in the product yield based on the dry biomass (Y_{P/X}). Compared to the strain according to the invention M. extorquens TK 0001+pTE1887-ghrA_{eco}, an 11% increase in this yield was achieved (Table 4).

The use of the exogenous codon-optimised ecmᵣₛₕ gene leads to the surprising deviations of the cultivation parameters in comparison to the strain according to the invention comprising the exogenous ecmₘₑₐ gene, as can be seen in Table 4. The highest measured lactic acid titer of 0.37 g L⁻¹ was observed here. A striking change concerns the dry-biomass-substrate-yield (Y_{X/S}), which is increased by 26% (0.24 g g_{dry biomass}⁻¹) compared to the strain according to the invention M. extorquens TK 0001+pTE1887-ghrA_{eco}-ecmₘₑₐ.

It was demonstrated that glycolic acid can be produced from methanol. The use of two exogenous ethylmalonyl-CoA mutase enzymes from two different prokaryotic strains increased the production performance of the production strains according to the invention compared to the strain according to the invention comprising ghrA_{eco} without an exogenous ethylmalonyl-CoA mutase. In particular, the use of the ethylmalonyl-Coa mutase ecmᵣₛₕ Surprisingly leads to a significantly increased and more selective lactic acid production.

TABLE 4
Summary of the cultivation parameters of M. extorquens TK 0001 + pTE1887 and M.
extorquens TK 0001 + pTE1887-ghrA_{eco} (codon-optimised) according to the invention
(M. extorquens GA1), M. extorquens TK 0001 + pTE1887-ghrA_{eco}-ecmₘₑₐ (ghrA_{eco}: codon-
optimised; ecmₘₑₐ: native) according to the invention (M. extorquens GA2) and M.
extorquens TK 0001 + pTE1887-ghrA_{eco}-ecmᵣₛₕ (both genes codon-optimised) according to
the invention (M. extorquens GA3) in culture medium with 10 g L⁻¹ methanol.

| | | Titer GA at | Y_{X/S} | Y_{P/S} | Y_{P/X} |
|---|---|---|---|---|---|
| | μ | approx. 40 | [g_{DBM} | [g_{GA} | [g_{GA} |
| strain and condition | [1 h⁻¹] | h [g L⁻¹] | g_{MeOH}⁻¹] | g_{MeOH}⁻¹] | g_{DBM}⁻¹] |
| M. extorquens AM1 + pTE1887 | 0.17 | 0.00 | 0.37 | 0.00 | 0.00 |
| +pTE1887- ghrA_{eco} (according to the invention) | 0.10 | 0.22 | 0.26 | 0.07 | 0.27 |
| +pTE1887-ghrA_{eco}-ecmₘₑₐ (according to the invention | 0.10 | 0.26 | 0.19 | 0.07 | 0.30 |
| +pTE1887-ghrA_{eco}-ecmᵣₛₕ (according to the invention) | 0.07 | 0.08 | 0.24 | 0.06 | 0.23 |

Abbreviations: μ, specific growth rate; MeOH, methanol; GA, glycolic acid; DBM, dry biomass.

### Example 5: Experimental Data of the Demonstration of the Enzyme Activity of the Glyoxylate Reductase (ghrA_{eco}) Expressed According to the Invention and of a Comparison Enzyme, Namely an E. coli Hydroxypyruvate Reductase (ghrB_{eco})

To provide experimental evidence for the presence of the enzyme activity of the expressed glyoxylate reductase ghrA_{eco} and the hydroxypyruvate reductase ghrB_{eco} (Nuñez, M. F., M. T. Pellicer, J. Badia, J. Aguilar, and L. Baldoma, Biochem J, 2001. 354 (Pt 3): p. 707-15, database entry for ghrA: https://biocyc.org/gene?orgid=ECOLI&id=G6539, database entry for ghrB: https://biocyc.org/gene?orgid=ECOLI&id=EG12272) in the strain background Methylorubrum extorquens TK 0001 enzyme assays were carried out. The native form of the DNA sequences (as found in Escherichia coli K-12 MG1655) and the synthetic DNA sequences (c-optimised) that are codon-optimised for expression in Methylobacteriaceae were tested to evaluate the influence of codon optimisation on gene expression and the resulting enzyme activity.

The procedure for carrying this out and the results are summarised below.

In order to obtain sufficient biomass of the genetically modified M. extorquens TK 0001 strains containing pTE1887-ghrA_{eco}-c-optimised (SEQ ID NO: 3), pTE1887-ghrB_{eco}-c-optimised, pTE1887-ghrA_{eco}-native (SEQ ID NO:1) and pTE1887-ghrB_{eco}-native for cell disruption, the following cultivation protocol was used. The strain M. extorquens TK 0001+pTE1887 containing the empty vector, was used as a negative control. All strains were cultivated, harvested and disrupted as three independent biological replicates.

The strains were cultivated for an initial three-day preculture (in minimal medium with methanol (see example 1) in baffled shake flasks (250 mL flask volume, 50 mL culture volume) at 30° C., 150 RPM and water vapour-saturated atmosphere (New Brunswick™ Innova 44, Eppendorf AG, Hamburg, Germany). Subsequently, a second preculture was inoculated from the first preculture in minimal medium with methanol in baffled shake flasks (250 mL flask volume, 50 mL culture volume). The initial biomass concentration used for the inoculation corresponded to an optical density at 600 nm (OD₆₀₀) of 0.1. The subsequent cultivation was carried out at 30° C., 150 rpm and in a water vapour-saturated atmosphere. On the next day, Tuesday, the main cultures were inoculated with the overgrown second precultures (50 mL minimal medium with methanol in 250 mL baffled shake flasks, initial OD₆₀₀=0.05) and incubated at 30° C., 150 rpm and in a water vapour-saturated atmosphere.

After the cultures had reached an OD₆₀₀ of 0.9-1.0, the gene expression of the glyoxylate reductases was induced with 1 mM IPTG (final concentration in the culture volume). Subsequently, the biomass was grown to a final OD600 of approx. 4-7.

For the actual biomass harvest, 50 mL conical centrifugation tubes were weighed empty, then each was filled with the 50 mL main culture and then centrifuged at 4,200 rpm for 15 min at 4° C. After centrifugation, the supernatant was discarded and the biomass pellets obtained were each washed with 20 mL of 50 mM Tris-HCl (pH 7.5) buffer. After that, centrifugation was repeated under the previous conditions, followed by careful removal of the supernatant with a pipette. The obtained biomass pellets were weighed and each was resuspended in 50 mM MOPS buffer (pH 6.6). For this, a buffer volume of 7 mL was used per 1 g wet pellet.

Cell disruption to obtain crude protein extracts containing the expressed glyoxylate reductases or hydroxypyruvate reductases was carried out in 2.0 mL reaction vessels. For this purpose, 1.5 mL of the cell suspension was transferred to each of these reaction vessels and subsequently disrupted six times for 30 seconds each at an amplitude of 60 by ultrasonication in an ice water bath. Between each of the six disruption cycles, the samples were cooled on ice for 1 minute. Finally, to obtain the raw protein extract, a centrifugation step at 21,500 rpm for 15 minutes at 4° C. was carried out. The protein-containing supernatant obtained was transferred to 1.5 mL reaction vessels. To ensure comparability of the enzyme assay results, the protein concentration of each raw protein extract was determined using a NanoDrop™. The raw extract with the lowest measured concentration was used as the target concentration for diluting the other raw extracts with 50 mM MOPS buffer (pH 6.6). This ensured that all crude protein extracts contained the same total protein concentration in the enzyme assay. Furthermore, these prediluted crude protein extracts were diluted a further time (1:5) with 50 mM MOPS buffer (pH 6.6) and subsequently used in the enzyme assay.

The enzyme assay was carried out in 96-well microtiter plates. For this, 160 μL of the diluted crude protein extracts were mixed with 20 μL of 50 mM glyoxylate as a substrate and 20 μL of a 2 mM cofactor stock solution (NADH or NADPH, final concentration in the assay 0.2 mM). The experimental approaches were carried out in three technical replicates. The enzyme activity was measured as the change in absorbance of NADH at 340 nm at 37° C. for up to 30 min. For the evaluation, the maximum change in absorbance over time was determined in the linear region of the reaction and multiplied by the dilution factor of five before calculating the enzyme activity in U mL⁻¹.

The enzyme activity was calculated using equation 6 and the given coefficients.

$$\frac{U}{{V}_{P roteinrohextrakt - A s s a y}} = S * \frac{{V}_{A s s a y}}{{ε}_{λ} * d * {V}_{P r oteinrohextrakt}} [ \frac{U}{mL} ] \qquad ( Equation 6 )$$

With enzyme activity: Measured in mol_{substrate} min⁻¹, V_{protein crude extract assay}: volume of protein crude extract used in the assay (0.00016 L), S: change in absorbance at 340 nm over time in the linear region of the reaction, corrected for the dilution factor of five (Abs.₃₄₀ min.⁻¹), V_{assay}: total volume of the assay (0.0002 L), ελ: extinction coefficient of NADH/NADPH at 340 nm (6220 L mol⁻¹ cm⁻¹), d: layer thickness of the absorbing reaction mixture (0.53 cm).

To convert the enzyme activity from mol_{substrate} min⁻¹ into the conventional unit for enzyme activity mU mL⁻¹ (1 U=1 μmol_{substrate} min⁻¹), the calculated result is multiplied by a factor of 10⁶.

The obtained enzyme activities were assigned to the respective expression strains and the cofactors NADH or NADPH used for a graphical comparison.

The data collected for the enzyme activities of ghrA_{eco}-c-optimised, ghrB_{eco}-c-optimised, ghrA_{eco}-native, ghrB_{eco}-native and the negative control (pTE1887 empty vector) are summarised in FIG. 12. The measurements and the standard deviation displayed are based on three biological replicates, each with three technical replicates of the assay.

As expected, the empty vector shows only a slight background activity. This was subtracted from all further measurements in order to correct for the background reaction that took place.

The enzyme activities with regard to the conversion of glyoxylate into glycolic acid shown in FIGS. 12A and 12B indicate that only the two enzymes used according to the invention, ghrA_{eco}-c-optimised and ghrA_{eco}-native (thus ghrA_{eco}), exhibit sufficient activity, particularly for large-scale production. Furthermore, significant differences in enzyme activity depending on the cofactor used can be demonstrated. The assay shows that there is a clear cofactor dependency of ghrA_{eco} and ghrB_{eco}. Using NADH as a cofactor (FIG. 12 A), the highest enzyme activity is achieved with ghrB_{eco}-c-optimised (10.53±1.50 mU mL⁻¹). The enzyme activity caused by the ghrA_{eco}-c-optimised gene with 0.49±2.34 mU mL⁻¹ is significantly reduced compared to ghrB_{eco}-c-optimised. A reduction in enzyme activity of around 95% was measured here. The enzyme activity of the NADH assays with the native genes is in a similar region: 4.61±1.61 mU mL⁻¹ versus 2.45±0.67 mU mL⁻¹ for ghrA_{eco}-native and ghrB_{eco}-native. Codon optimisation of ghrB_{eco} leaded to an increase in activity of 329%. In summary, a clear dependence of the ghrB_{eco} enzyme on NADH as a cofactor can be recognized.

In contrast, a different picture emerged when using NADPH as a cofactor (FIG. 12 B).

Here, ghrA_{eco}-c-optimised and ghrA_{eco}-native (33.86±1.29 mU mL⁻¹ and 21.76±1.49 mU mL⁻¹) achieve by far the highest enzyme activities measured in the tests that were present. The increase due to codon optimisation is 55% (21.76±1.49 versus 33.86±1.29 mU mL⁻¹). It can also be clearly verified that the ghrA_{eco} enzyme has an NADPH dependency. This is underlined by the low measured activity of ghrB_{eco}. In this case, for both the use of the condon-optimised variant of the gene (ghrB_{eco}-c-optimised) and the native variant of the gene (ghrB_{eco}-native) only a low enzyme activity of 3.17±0.29 and 0.81±0. 0.30 mU mL⁻¹ (FIG. 12 B) was measured, which shows that ghrB_{eco} can be clearly distinguished from ghrA not only with regard to the observed very low NADPH dependence, but primarily also with regard to the low enzyme activity in the conversion of glyoxylate into glycolic acid.

The increased enzyme activity with NADPH as cofactor triggered by the expression of ghrA_{eco}-c-optimised shows that the production of glycolic acid by M. extorquens is possible through the expression of this enzyme. The significantly reduced enzyme activity measured in the context of ghrB_{eco}-c-optimised with both NADPH and NADH is not sufficient to enable glycolic acid production in vivo in M. extorquens.

The glycolic acid production observed with M. extorquens TK 0001+pTE1887-ghrA_{eco}-c-optimised appears to be dependent on the availability of the cofactor NADPH. These results confirm the results from example 2.

Surprisingly, introducing the DNA sequence of the ghrA_{eco} enzyme, in particular the codon-optimised DNA sequence, leads to a glycolic acid production as well as the surprising production of lactic acid.

### Example 6

Expression of exogenous nucleic acid sequence encoding a glyoxylate reductase from the bacterium Escherichia in cells of further Methylobacteriaceae (invention) and other microorganisms (comparison)

Further genera of the family Methylobacteriaceae (Alphaproteobacteria) were genetically modified according to Example 1. FIG. 13 shows the microorganisms examined as examples. In particular, the following were examined as representatives of the Methylobacteriaceae: Methylorubrum, in particular M. zatmanii DSM 5688, in particular M. extorquens TK 0001 DSM 1337 (examples 2 to 5), in particular M. extorquens PA1 DSM 23939, in particular M. rhodesianum DSM 5687, a derivative of M. extorquens AM1 DSM 1338 with a deletion of a cellulase gene (M. extorquens AM1Δcel: https://doi.org/10.1371/journal.pone.0062957), and Methylobacterium cells, in particular M. organophilum DSM 18172, in particular M. radiotolerans DSM 760.

In addition, Methylomonas methanica DSM 25384 (Gammaproteobacteria), Methylophilus methylotrophus DSM 6330 (Betaproteobacteria) and Bacillus methanolicus DSM 16454 (Firmicutes) were examined as negative examples not belonging to the family Methylobacteriaceae. The aforementioned microorganisms are also able to metabolise methanol and were tested for the production of glycolic acid and/or lactic acid according to the invention.

The following expression vectors (1 to 4) were used:
1.) pTE1887 (expression vector, also named as empty vector; plasmid map: FIG. 8)
2) pTE1887-ghrA_{eco} (expression vector which codon-optimised encodes the glyoxylate reductase from Escherichia coli K-12 MG1655; with SEQ ID NO: 3, plasmid map: FIG. 9) (according to the invention)
3) pTE1887-ghrA_{eco}-ecmₘₑₐ (expression vector which encodes the glyoxylate reductase from Escherichia coli K-12 MG1655 (codon-optimised) and the ethylmalonyl-CoA mutase from M. extorquens TK 0001 DSM 1337 natively; plasmid map: FIG. 10) (according to the invention)
4) pTE1887-ghrA_{eco}-ecmᵣₛₕ (expression vector encoding the glyoxylate reductase from Escherichia coli K-12 MG1655 (codon-optimised) and the ethylmalonyl-CoA mutase from Rhodobacter sphaeroides ATCC 17029 codon-optimised; plasmid map: FIG. 11) (according to the invention).

Genetically modified Methylobacteriaceae cells comprising an exogenous, codon-optimised nucleic acid sequence (SEQ ID NO: 3) encoding a glyoxylate reductase from the bacterium Escherichia coli K-12 MG1655 of the strain Methylorubrum zatmanii Mza-GA14 (M. zatmanii DSM 5688+pTE1887-ghrA_{eco}) were deposited on 19 Jul. 2023 at the DSMZ, German Collection of Microorganisms and Cell Cultures, Braunschweig, Germany under the deposit number DSM 34701.

Genetically modified Methylobacteriaceae cells comprising an exogenous, a glyoxylate reductase from the bacterium Escherichia coli K-12 MG1655 encoding codon-optimised nucleic acid sequence (SEQ ID NO: 3) of the strain Methylorubrum extorquens Mea-GA17 (M. extorquens PA1 DSM 23939+pTE1887-ghrA_{eco}) were deposited on 19 Jul. 2023 at the DSMZ, German Collection of Microorganisms and Cell Cultures, Braunschweig, Germany under the deposit number DSM 34702.

Genetically modified Methylobacteriaceae cells comprising an exogenous, codon-optimised nucleic acid sequence (SEQ ID NO: 3) encoding a glyoxylate reductase from the bacterium Escherichia coli K-12 MG1655 of the strain Methylorubrum rhodesianum Mrh-GA4 (M. rhodesianum DSM 5687+pTE1887-ghrA_{eco}) were deposited on 19 Jul. 2023 at DSMZ, German Collection of Microorganisms and Cell Cultures, Braunschweig, Germany under deposit number DSM 34697.

Genetically modified Methylobacteriaceae cells comprising an exogenous, codon-optimised nucleic acid sequence (SEQ ID NO: 3) encoding a glyoxylate reductase from the bacterium Escherichia coli K-12 MG1655 and an exogenous, native nucleic acid sequence (SEQ ID NO: 4) encoding an ethylmalonyl-CoA mutase from the bacterium Methylorubrum extorquens TK 0001 DSM 1337 of the strain Methylorubrum rhodesianum Mrh-GA5 (M. rhodesianum hodesianum DSM 5687+pTE1887-ghrA_{eco}-ecmₘₑₐ) were deposited on 19 Jul. 2023 at the DSMZ, German Collection of Microorganisms and Cell Cultures, Braunschweig, Germany under the deposit number DSM 34698.

Genetically modified Methylobacteriaceae cells comprising an exogenous, codon-optimised nucleic acid sequence (SEQ ID NO: 3) encoding a glyoxylate reductase from the bacterium Escherichia coli K-12 MG1655 of the strain Methylobacterium organophilum Mor-GA 8 (M. organophilum DSM 18172+pTE1887-ghrA_{eco}-ecmₘₑₐ) were deposited on 19 Jul. 2023 at the DSMZ, German Collection of Microorganisms and Cell Cultures, Braunschweig, Germany under the deposit number DSM 34699.

Genetically modified Methylobacteriaceae cells comprising an exogenous, codon-optimised nucleic acid sequence (SEQ ID NO: 3) encoding a glyoxylate reductase from the bacterium Escherichia coli K-12 MG1655 and an exogenous, native nucleic acid sequence (SEQ ID NO: 4) encoding a ethylmalonyl-CoA mutase from the bacterium Methylorubrum extorquens TK 0001 DSM 1337 of the strain Methylobacterium radiotolerans Mra-GA12 (M. radiotolerans DSM 760+pTE1887-ghrA_{eco}-ecmₘₑₐ) were deposited on 19 Jul. 2023 at the DSMZ, German Collection of Microorganisms and Cell Cultures, Braunschweig, Germany under the deposit number DSM 34700.

To study the invention with the aforementioned strains, the process was carried out according to example 2. In contrast to example 2, the cultivations with the Methylobacteriaceae cells M. rhodesianum (FIG. 14) DSM 5687, M. zatmanii DSM 5688 (FIG. 15), M. radiotolerans DSM 760 (FIG. 16), M. organophilum DSM 18172 (FIG. 17), M. extorquens PA1 DSM 23939 (FIG. 18) were started with a reduced amount of reactant (Cx compound, 4 g L⁻¹ methanol) and additionally reactant was fed between ten and twelve hours after induction (fed-batch, cumulatively up to 15 g L⁻¹). Furthermore, the samples were withdrawn to determine the concentrations of glycolic acid, lactic acid and methanol after 22-28 h after induction of gene expression with 1 mM IPTG.

FIGS. 14 to 19 show the genetically modified Methylobacteriaceae cells on the x-axis and the concentration of methanol (white, unfilled bar) or the concentration of the mixture of formed glycolic acid and lactic acid (black, filled bar) in g L⁻¹ in the reaction medium on the y-axis. All sample taking times are 22 to 28 hours after induction of gene expression with 1 mM IPTG. All concentrations are given in g L⁻¹, determined by HPLC, refractive index detection and external standards.

FIG. 14 shows the screening result of glycolic acid and lactic acid production with recombinant M. rhodesianum DSM 5687 strains expressing glyoxylate reductases based on the corresponding codon-optimised genes. The first entry from the left shows the methanol concentration in the minimal medium at the beginning of cultivation. pTE1887 was used as the expression vector, which also serves as a negative control in the form of the empty vector in the reference strain M. rhodesianum DSM 5687+pTE1887 (second entry from the left on the x-axis).

Surprisingly, both the reference strain M. rhodesianum DSM 5687+pTE1887 (second entry from the left) and the genetically modified Methylobacteriaceae cells showed no glycolic acid or lactic acid production (entries from the left: 3 to 10 and 12 to 16), with the exception (black, filled bars in FIG. 14) of the genetically modified cells of M. rhodesianum DSM 5687+pTE1887-ghrA_{eco} (in codon-optimised nucleic acid form according to SEQ ID NO: 3) according to the invention, thus a genetically modified Methylobacteriaceae cell according to the invention comprising at least one exogenous nucleic acid sequence encoding a glyoxylate reductase from the bacterium Escherichia (entry from the left: 11) and the genetically modified cells M. rhodesianum DSM 5687+pTE1887-ghrA_{eco}-ecmₘₑₐ comprising an exogenous, codon-optimised nucleic acid sequence (SEQ ID NO: 3) encoding a glyoxylate reductase from the bacterium Escherichia coli K-12 MG1655 and an exogenous native nucleic acid sequence (SEQ ID NO: 4) encoding an ethylmalonyl-CoA mutase from the bacterium Methylorubrum extorquens TK 0001 DSM 1337, thus a genetically modified Methylobacteriaceae cell according to the invention comprising at least one exogenous nucleic acid sequence encoding a glyoxylate reductase from the bacterium Escherichia (entry from the left: 17) and the genetically modified cells M. rhodesianum DSM 5687+pTE1887-ghrA_{eco}-ecmᵣₛₕ, comprising an exogenous codon-optimised nucleic acid sequence (SEQ ID NO: 3) encoding a glyoxylate reductase from the bacterium Escherichia coli K-12 MG1655 and an exogenous codon-optimised nucleic acid sequence (SEQ ID NO: 8) encoding an ethylmalonyl-CoA mutase from the bacterium Rhodobacter sphaeroides ATCC 17029, thus a genetically modified Methylobacteriaceae cell according to the invention comprising at least one exogenous nucleic acid sequence encoding a glyoxylate reductase from the bacterium Escherichia (entry from the left: 18).

FIG. 9 shows the map of the vector used to generate these Methylobacteriaceae cells, with the following elements: lacI gene, lacI promoter, PL/O4/A1 promoter→−33 region→−10 region→transcription start, PL/O4/A1 promoter ribosomal binding site (RBS), ghrA_{eco}-c optimised, Lambda T0 terminator, kanamycin resistance, mobilisation genes mobS and mob, regulatory protein RepA. Origin of replication colE1.

FIG. 10 shows the map of the vector used to generate these Methylobacteriaceae cells expressing ghrA_{eco}-ecmₘₑₐ with the following elements: lacI gene, lacI promoter, PL/O4/A1 promoter→−33 region→−10 region→transcription start, PL/O4/A1 promoter ribosomal binding site (RBS), ghrA_{eco} (codon-optimised), ecmₘₑₐ (native), lambda T0 terminator, kanamycin resistance, mobilisation genes mobS and mob, regulatory protein RepA. Origin of replication colE1.

FIG. 11 shows the map of the vector used to generate these Methylobacteriaceae cells, expressing ghrA_{eco}-ecmᵣₛₕ, with the following elements: lacI gene, lacI promoter, PL/O4/A1 promoter→−33 region→−10 region→transcription start, PL/O4/A1 promoter ribosomal binding site (RBS), ghrA_{eco} (codon-optimised), rsh-ecm (codon-optimised), lambda T0 terminator, kanamycin resistance, mobilisation genes mobS and mob, regulatory protein RepA. Origin of replication colE1.

The cells of M. rhodesianum DSM 5687 genetically modified according to the invention were able to produce mixtures of glycolic acid and lactic acid containing a total concentration of glycolic acid plus lactic acid of up to 0.85 g L⁻¹ (M. rhodesianum DSM5687+pTE1887-ghrA_{eco}-ecmₘₑₐ), at least 0.82 g L⁻¹ (M. rhodesianum DSM5687+pTE1887-ghrA_{eco}), at least 0.09 g L⁻¹ (M. rhodesianum DSM5687+pTE1887-ghrA_{eco}-ecmᵣₛₕ).

These experimental data demonstrate that glycolic acid and lactic acid production according to the invention is possible within the family Methylobacteriaceae.

FIG. 15 shows the screening result of glycolic acid and lactic acid production with recombinant M. zatmanii DSM 5688 strains which, according to the invention, express the gene for glyoxylate reductase from Escherichia and, in one case, additionally the gene of an ethylmalonyl-CoA mutase from Rhodobacter sphaeroides ATCC 17029, based on the corresponding codon-optimised genes. The first entry from the left shows the methanol concentration in the minimal medium at the beginning of cultivation. pTE1887 was used as the expression vector, which also serves as a negative control in the form of the empty vector in the reference strain M. zatmanii DSM 5688+pTE1887 (second entry from the left on the x-axis).

Surprisingly, the reference strain M. zatmanii DSM 5688+pTE1887 (second entry from the left) showed no glycolic acid and lactic acid production, in contrast to (black, filled bars in FIG. 15) the genetically modified cells of M. zatmanii DSM 5688+pTE1887-ghrA_{eco} according to the invention (in codon-optimised nucleic acid form according to SEQ ID NO: 3), thus a genetically modified Methylobacteriaceae cell according to the invention comprising at least one exogenous nucleic acid sequence encoding a glyoxylate reductase from the bacterium Escherichia (entry from the left: 3) and the genetically modified cells M. zatmanii DSM 5688+pTE1887-ghrA_{eco}-ecmᵣₛₕ comprising an exogenous, codon-optimised nucleic acid sequence (SEQ ID NO: 3) encoding a glyoxylate reductase from the bacterium Escherichia coli K-12 MG1655 and an exogenous, codon-optimised nucleic acid sequence (SEQ ID NO: 8) encoding an ethylmalonyl-CoA mutase from the bacterium Rhodobacter sphaeroides ATCC 17029, thus a genetically modified Methylobacteriaceae cell according to the invention comprising at least one exogenous nucleic acid sequence encoding a glyoxylate reductase from the bacterium Escherichia (entry from the left: 4) FIG. 9 shows the map of the vector used to generate these Methylobacteriaceae cells, with the following elements: lacI gene, lacI promoter, PL/O4/A1 promoter→−33 region→−10 region 4 transcription start, PL/O4/A1 promoter ribosomal binding site (RBS), ghrA_{eco}-c optimised, Lambda T0 terminator, kanamycin resistance, mobilisation genes mobS and mob, regulatory protein RepA. Origin of replication colE1.

FIG. 11 shows the map of the vector used to generate these Methylobacteriaceae cells, expressing ghrA_{eco}-ecmᵣₛₕ, with the following elements: lacI gene, lacI promoter, PL/O4/A1 promoter→−33 region→−10 region→transcription start, PL/O4/A1 promoter ribosomal binding site (RBS), ghrA_{eco} (codon-optimised), rsh-ecm (codon-optimised), lambda T0 terminator, kanamycin resistance, mobilisation genes mobS and mob, regulatory protein RepA. Origin of replication colE1.

Mixtures of glycolic acid and lactic acid could be produced from the genetically modified cells of M. zatmanii DSM 5688 according to the invention, containing a total concentration of glycolic acid plus lactic acid of up to 0.56 g L⁻¹ (M. zatmanii DSM 5688+pTE1887-ghrA_{eco}), at least 0.48 g L⁻¹ (M. zatmanii DSM 5688+pTE1887-ghrA_{eco}-ecmᵣₛₕ).

These experimental data demonstrate that glycolic acid and lactic acid production according to the invention is possible within the family of Methylobacteriaceae.

FIG. 16 shows the screening result of glycolic acid and lactic acid production with a recombinant M. radiotolerans DSM 760 strain. The first entry from the left shows the methanol concentration in the minimal medium at the beginning of cultivation. pTE1887 was used as expression vector, which also serves as a negative control in the form of the empty vector in the reference strain M. radiotolerans DSM 760+pTE1887 (second entry from the left on the x-axis).

Surprisingly, the reference strain M. radiotolerans DSM 760+pTE1887 (second entry from the left) showed no glycolic acid or lactic acid production, in contrast to the genetically modified cells of M. radiotolerans DSM 760+pTE1887-ghrA_{eco}-ecmₘₑₐ according to the invention comprising an exogenous, codon-optimised nucleic acid sequence (SEQ ID NO: 3) encoding a glyoxylate reductase from the bacterium Escherichia coli K-12 MG1655 and an exogenous, native nucleic acid sequence (SEQ ID NO: 4) encoding an ethylmalonyl-CoA mutase from the bacterium Methylorubrum extorquens TK 0001 DSM 1337, thus a genetically modified Methylobacteriaceae cell according to the invention comprising at least one exogenous nucleic acid sequence encoding a glyoxylate reductase from the bacterium Escherichia (entry from the left: 3) (black filled bar in FIG. 16).

The combination of ghrA_{eco} and ecmₘₑₐ (an ethylmalonyl-CoA mutase from M. extorquens TK 0001 DSM 1337 according to the invention) leaded to the production of glycolic acid and lactic acid according to the invention.

FIG. 10 shows the map of the vector used to generate these Methylobacteriaceae cells expressing ghrA_{eco}-ecmₘₑₐ with the following elements: lacI gene, lacI promoter, PL/O4/A1 promoter→−33 region→−10 region→transcription start, PL/O4/A1 promoter ribosomal binding site (RBS), ghrA_{eco} (codon-optimised), ecmₘₑₐ (native), lambda T0 terminator, kanamycin resistance, mobilisation genes mobS and mob, regulatory protein RepA. Origin of replication colE1. Mixtures of glycolic acid and lactic acid could be produced with the genetically modified cells of M. radiotolerans DSM 760 according to the invention containing a total concentration of glycolic acid plus lactic acid of up to 0.39 g L⁻¹ (M. radiotolerans DSM 760+pTE1887-ghrA_{eco}-ecmₘₑₐ).

These experimental data demonstrate that glycolic acid and lactic acid production according to the invention is possible within the family of Methylobacteriaceae.

FIG. 17 shows the screening result of glycolic acid and lactic acid production with recombinant M. organophilum DSM 18172 strains expressing glyoxylate reductases based on the corresponding codon-optimised genes. The first entry from the left shows the methanol concentration in the minimal medium at the beginning of cultivation. pTE1887 was used as expression vector, which also serves as negative control in form of the empty vector form in the reference strain M. organophilum DSM 18172+pTE1887 (second entry from the left on the x-axis).

Surprisingly, neither the reference strain M. organophilum DSM 18172+pTE1887 (second entry from the left) nor the genetically modified Methylobacteriaceae cells showed any glycolic acid or lactic acid production (entries from the left: 3 to 10 and 12 to 18), with the exception (black, filled bars in FIG. 17) of the genetically modified cells of M. organophilum DSM 18172+pTE1887-ghrA_{eco} (in codon-optimised nucleic acid form according to SEQ ID NO: 3) according to the invention, thus a genetically modified Methylobacteriaceae cell according to the invention comprising at least one exogenous nucleic acid sequence encoding a glyoxylate reductase from the bacterium Escherichia (entry from the left: 11) and the genetically modified cells M. organophilum DSM 18172+pTE1887-ghrA_{eco}-ecmₘₑₐ comprising an exogenous codon-optimised nucleic acid sequence (SEQ ID NO: 3) encoding a glyoxylate reductase from the bacterium Escherichia coli K-12 MG1655 and an exogenous native nucleic acid sequence (SEQ ID NO: 4) encoding an ethylmalonyl-CoA mutase from the bacterium Methylorubrum extorquens TK 0001 DSM 1337, thus a genetically modified Methylobacteriaceae cell according to the invention comprising at least one exogenous nucleic acid sequence encoding a glyoxylate reductase from the bacterium Escherichia (entry from left: 17) and the genetically modified cells M. organophilum DSM 18172+pTE1887-ghrA_{eco}-ecmᵣₛₕ comprising an exogenous codon-optimised nucleic acid sequence (SEQ ID NO: 3) encoding a glyoxylate reductase from the bacterium Escherichia coli K-12 MG1655 and an exogenous codon-optimised nucleic acid sequence (SEQ ID NO: 8) encoding an ethylmalonyl-CoA mutase from the bacterium Rhodobacter sphaeroides ATCC 17029, thus a genetically modified Methylobacteriaceae cell according to the invention comprising at least one exogenous nucleic acid sequence encoding a glyoxylate reductase from the bacterium Escherichia (entry from the left: 18).

FIG. 9 shows the map of the vector used to generate these Methylobacteriaceae cells, with the following elements: lacI gene, lacI promoter, PL/O4/A1 promoter→−33 region→−10 region 4 transcription start, PL/O4/A1 promoter ribosomal binding site (RBS), ghrA_{eco}-c optimised, Lambda T0 terminator, kanamycin resistance, mobilisation genes mobS and mob, regulatory protein RepA. Origin of replication colE1.

FIG. 10 shows the map of the vector used to generate these Methylobacteriaceae cells expressing ghrA_{eco}-ecmₘₑₐ with the following elements: lacI gene, lacI promoter, PL/O4/A1 promoter→−33 region→−10 region→transcription start, PL/O4/A1 promoter ribosomal binding site (RBS), ghrA_{eco} (codon-optimised), ecmₘₑₐ (native), lambda T0 terminator, kanamycin resistance, mobilisation genes mobS and mob, regulatory protein RepA. Origin of replication colE1.

FIG. 11 shows the map of the vector used to generate these Methylobacteriaceae cells, expressing ghrA_{eco}-ecmᵣₛₕ, with the following elements: lacI gene, lacI promoter, PL/O4/A1 promoter→−33 region→−10 region→transcription start, PL/O4/A1 promoter ribosomal binding site (RBS), ghrA_{eco} (codon-optimised), rsh-ecm (codon-optimised), lambda T0 terminator, kanamycin resistance, mobilisation genes mobS and mob, regulatory protein RepA. Origin of replication colE1.

Mixtures of glycolic acid and lactic acid could be produced from the genetically modified cells of M. organophilum DSM 18172 according to the invention, containing a total concentration of glycolic acid plus lactic acid of up to 0.13 g L⁻¹ (M. organophilum DSM 18172+pTE1887-ghrA_{eco}), at least 0.10 g L⁻¹ (M. organophilum DSM 18172+pTE1887-ghrA_{eco}-ecmₘₑₐ), at least 0.04 g L⁻¹ (M. organophilum DSM 18172+pTE1887-ghrA_{eco}-ecmᵣₛₕ).

These experimental data demonstrate that glycolic acid and lactic acid production according to the invention is possible within the family of Methylobacteriaceae.

FIG. 18 shows the screening result of glycolic acid and lactic acid production with recombinant M. extorquens PA1 DSM 23939 strains. The first entry from the left shows the methanol concentration in the minimal medium at the beginning of cultivation. pTE1887 was used as expression vector, which also serves as a negative control in the form of the empty vector in the reference strain M. extorquens PA1 DSM 23939+pTE1887 (second entry from the left on the x-axis).

Surprisingly, the reference strain M. extorquens PA1 DSM 23939+pTE1887 (second entry from the left) showed no glycolic acid or lactic acid production, in contrast to the genetically modified cells of M. extorquens PA1 DSM 23939+pTE1887-ghrA_{eco} (in codon-optimised nucleic acid form according to SEQ ID NO: 3) according to the invention, thus a genetically modified Methylobacteriaceae cell according to the invention comprising at least one exogenous nucleic acid sequence encoding a glyoxylate reductase from the bacterium Escherichia (black filled bar in FIG. 18) (entry from the left: 3).

FIG. 9 shows the map of the vector used to generate these Methylobacteriaceae cells, with the following elements: lacI gene, lacI promoter, PL/O4/A1 promoter→−33 region→−10 region 4 transcription start, PL/O4/A1 promoter ribosomal binding site (RBS), ghrA_{eco}-c optimised, Lambda T0 terminator, kanamycin resistance, mobilisation genes mobS and mob, regulatory protein RepA. Origin of replication colE1.

Mixtures of glycolic acid and lactic acid could be produced from the genetically modified cells M. extorquens PA1 DSM 23939 according to the invention, containing a total concentration of glycolic acid plus lactic acid up to 1.50 g L⁻¹ (M. extorquens PA1 DSM 23939+pTE1887-ghrA_{eco}) (M. extorquens GA17).

These experimental data demonstrate that the production of glycolic acid and lactic acid according to the invention is possible within the family Methylobacteriaceae.

FIG. 19 shows the screening results of glycolic acid and lactic acid production with recombinant M. extorquens AM1Δcel strains which express glyoxylate reductases based on the corresponding codon-optimised genes. The first entry from the left shows the methanol concentration in the minimal medium at the beginning of cultivation. pTE1887 was used as expression vector, which also serves as a negative control in the form of the empty vector in the reference strain M. extorquens AM1Δcel+pTE1887 (second entry from the left on the x-axis).

Surprisingly, the reference strain M. extorquens AM1Δcel+pTE1887 (second entry from the left) showed no glycolic acid or lactic acid production, with the exception (black filled bar in FIG. 19) of the genetically modified M. extorquens AM1Δ cel+pTE1887-ghrA_{eco} (in codon-optimised nucleic acid form according to SEQ ID NO: 3) according to the invention, thus a genetically modified Methylobacteriaceae cell according to the invention comprising at least one exogenous nucleic acid sequence encoding a glyoxylate reductase from the bacterium Escherichia (entry from the left: 11).

FIG. 9 shows the map of the vector used to generate these Methylobacteriaceae cells, with the following elements: lacI gene, lacI promoter, PL/O4/A1 promoter→−33 region→−10 region 4 transcription start, PL/O4/A1 promoter ribosomal binding site (RBS), ghrA_{eco}-c optimised, Lambda T0 terminator, kanamycin resistance, mobilisation genes mobS and mob, regulatory protein RepA. Origin of replication colE1.

Mixtures of glycolic acid and lactic acid could be produced from the genetically modified M. extorquens AM1Δcel cells according to the invention, containing a total concentration of glycolic acid plus lactic acid of up to 0.60 g L⁻¹ (M. extorquens AM1Δcel+pTE1887-ghrA_{eco}).

These experimental data demonstrate that glycolic acid and lactic acid production according to the invention is possible within the family of Methylobacteriaceae. Deletion of the cellulase gene (Δcel) does not affect glyoxylate-glycolic acid-lactic acid-metabolism.

The results of these studies are summarised in Table 5 (below).

Further studies were carried out in methylotrophic microorganisms not belonging to the family Methylobacteriaceae. For this purpose, the strain construction procedures according to example 1 were carried out to generate genetically modified strains of Methylomonas methanica DSM 25384 (Gammaproteobacteria), Methylophilus methylotrophus DSM 6330 (Betaproteobacteria) and Bacillus methanolicus DSM 16454 (Firmicutes). This was not possible in any of the cases with the strains used. The strains studied showed no growth during the strain construction procedure described in example 1 for all vectors pTE1887, pTE1887-ghrA_{eco}, pTE1887-ghrA_{eco}-ecmₘₑₐ and pTE1887-ghrA_{eco}-ecmᵣₛₕ (Table 5).

TABLE 5
Summary of the achieved glycolic acid and lactic acid titers of tested
strains of the family Methylobacteriaceae and comparative examples (microorganisms
not belonging to the family of the Methylobacteriaceae).

| | Strain growth on MO | | Titer Ga + | |
|---|---|---|---|---|
| | medium with 0.5% | Growth after | LA after | |
| | methanol, Kanamycin | introducing | 22 h to 28 | |
| strain | 30 (μg/mL), 30° C.^{[1]} | plasmids | h [g L⁻¹] | Deposited at DSMZ |
| Methylorubrum rhodesianum | yes | yes | 0.82 | M. rhodesianum |
| DSM 5687 + pTE1887- | | | | Mrh-GA4 (DSM |
| ghrA_{eco} | | | | 34697) |
| Methylorubrum rhodesianum | yes | yes | 0.85 | M. rhodesianum |
| DSM 5687 + pTE1887- | | | | Mrh-GA5 (DSM |
| ghrAeco-ecmₘₑₐ | | | | 34698) |
| Methylorubrum rhodesianum | yes | yes | 0.09 | no |
| DSM 5687 + pTE1887- | | | | |
| ghrA_{eco}-ecmᵣₛₕ | | | | |
| Methylorubrum zatmanii | yes | yes | 0.56 | M. zatmanii Mza- |
| DSM 5688 + pTE1887- | | | | GA14 (DSM 34701) |
| ghrA_{eco} | | | | |
| Methylorubrum zatmanii | yes | yes | 0.48 | no |
| DSM 5688 + pTE1887- | | | | |
| ghrA_{eco}-ecmᵣₛₕ | | | | |
| Methylobacterium | yes | yes | 0.39 | M. radiotolerans |
| radiotolerans DSM 760 + | | | | Mra-GA12 (DSM |
| pTE1887-ghrA_{eco}-ecmₘₑₐ | | | | 34700) |
| Methylobacterium | yes | yes | 0.13 | M. organophilum |
| organophilum DSM 18172 + | | | | Mor-GA8 (DSM |
| pTE1887-ghrA_{eco} | | | | 34699) |
| Methylobacterium | yes | yes | 0.04 | no |
| organophilum DSM 18172 + | | | | |
| pTE1887-ghrA_{eco}-ecmₘₑₐ | | | | |
| Methylobacterium | yes | yes | 0.10 | no |
| organophilum DSM 18172 + | | | | |
| pTE1887-ghrA_{eco}-ecmᵣₛₕ | | | | |
| Methylorubrum extorquens | yes | yes | 1.50 | M. extorquens Mea- |
| PA1 DSM 23939 + | | | | GA17 (DSM 34702) |
| pTE1887-ghrA_{eco} | | | | |
| Methylorubrum extorquens | yes | yes | 0.60 | no |
| AM1Δcel + pTE1887- | | | | |
| ghrA_{eco} | | | | |
| Methylomonas methanica | no | — | — | — |
| DSM 25384 | | | | |
| Methylophilus | yes^{[1]} | no | — | — |
| methylotrophus DSM 6330 | | | | |
| Bacillus methanolicus | no^{[1]} | — | — | — |
| DSM 16454 | | | | |

^{[1]}Temperatures used in growth tests: Methylophilus methylotrophus DSM 6330 (37° C.), Bacillus methanolicus DSM 16454 (45° C.).
Abbreviations: GA, glycolic acid; LA, lactic acid.

1. A genetically modified Methylobacteriaceae cell comprising at least one exogenous nucleic acid sequence encoding a glyoxylate reductase from the bacterium Escherichia.

2. The genetically modified cell of claim 1, wherein the Methylobacteriaceae cell is a Methylorubrum cell, in particular a cell of Methylorubrum extorquens, in particular Methylorubrum extorquens AM1, Methylorubrum extorquens TK 0001, Methylorubrum extorquens PA1, Methylorubrum rhodesianum or Methylorubrum zatmanii, or a Methylobacterium cell, in particular a cell of Methylobacterium organophilum or Methylobacterium radiotolerans.

3. The genetically modified Methylobacteriaceae cell of claim 1, wherein the bacterium is Escherichia coli, in particular E. coli K-12 MG1655.

4. The genetically modified Methylobacteriaceae cell of claim 1, wherein the glyoxylate reductase from the bacterium Escherichia is encoded by a nucleic acid sequence according to SEQ ID No. 3 or a functional equivalent thereof, wherein the functional nucleic acid sequence equivalent has a nucleic acid sequence identity of 30.0 to 99.9% to the nucleic acid sequence according to SEQ ID No. 3, or wherein the glyoxylate reductase has an amino acid sequence according to SEQ ID No. 2 or a functional amino acid sequence equivalent thereof, wherein the functional amino acid sequence equivalent has an amino acid sequence identity of 30.0 to 99.9% to the amino acid sequence of SEQ ID No. 2.

5. The genetically modified Methylobacteriaceae cell of claim 1, comprising at least one exogenous nucleic acid sequence that encodes an ethylmalonyl-CoA mutase, in particular from at least one bacterium selected from the group consisting of Methylorubrum extorquens, in particular Methylorubrum extorquens TK 0001 DSM 1337, and Rhodobacter sphaeroides, in particular Rhodobacter sphaeroides ATCC 17029.

6. The genetically modified Methylobacteriaceae cell of claim 5, wherein the ethylmalonyl-CoA mutase is encoded by a nucleic acid sequence according to SEQ ID No. 8 or 13 or a functional equivalent thereof, wherein the functional nucleic acid sequence equivalent has a nucleic acid sequence identity of 30.0 to 99.9% to the nucleic acid sequence according to SEQ ID No. 8 or 13, or wherein the ethylmalonyl-CoA mutase has an amino acid sequence according to SEQ ID No. 5 or 7, or a functional equivalent thereof, wherein the functional amino acid sequence equivalent has an amino acid sequence identity of 30.0 to 99.9% to the amino acid sequence according to SEQ ID No. 5 or 7.

7. The genetically modified Methylobacteriaceae cell of claim 1, wherein the at least one exogenous nucleic acid sequence encoding the glyoxylate reductase and/or encoding the ethylmalonyl-CoA mutase is integrated into the chromosome of the Methylobacteriaceae cell or is present extrachromosomally, in particular is present in the cell integrated in an episomal expression vector.

8. The genetically modified Methylobacteriaceae cell of claim 1, wherein the genetically modified Methylobacteriaceae cell is a cell of the Methylorubrum strain Methylorubrum extorquens Mea-GA1, (DSM 34286), Methylorubrum extorquens Mea-GA2, (DSM 34287) or Methylorubrum extorquens Mea-GA3 (DSM 34288), each deposited on 10 Jun. 2022 at the DSMZ, German Collection of Microorganisms and Cell Cultures, Braunschweig, Germany, or a derivative thereof, or wherein the genetically modified Methylobacteriaceae cell is a cell of the Methylorubrum strain Methylorubrum rhodesianum Mrh-GA4 (DSM 34697), Methylorubrum rhodesianum Mrh-GA5 (DSM 34698), Methylorubrum zatmanii Mza-GA14 (DSM 34701), Methylorubrum extorquens Mea-GA17 (DSM 34702) or a cell of the Methylobacterium strain Methylobacterium radiotolerans Mra-GA12 (DSM 34700) or Methylobacterium organophilum Mor-GA8 (DSM 34699), each deposited on 19 Jul. 2023 at the DSMZ, German Collection of Microorganisms and Cell Cultures, Braunschweig, Germany, or a derivative thereof.

9. The genetically modified Methylobacteriaceae cell of o claim 1, wherein the at least one exogenous nucleic acid sequence encoding glyoxylate reductase and/or encoding ethylmalonyl-CoA mutase is functionally connected to additionally at least one regulatory unit by forming an expression cassette, in particular a promoter, in particular an inducible, derepressible or constitutive promoter, an enhancer, a ribosomal binding site and/or a terminator.

10. A process for producing a genetically modified Methylobacteriaceae cell of claim 1, comprising the process steps of:
a) providing a Methylobacteriaceae cell, in particular a wild-type cell, and an expression vector or a genome editing system comprising at least one exogenous nucleic acid sequence encoding a glyoxylate reductase from the bacterium Escherichia, in particular an expression cassette comprising this nucleic acid sequence,
b) transforming the Methylobacteriaceae cell with the expression vector or the genome editing system under conditions that enable the uptake and, optionally stable, integration of the at least one exogenous nucleic acid sequence into the Methylobacteriaceae cell, and
c) obtaining the genetically modified Methylobacteriaceae cell having at least one exogenous, glyoxylate reductase.

11. The process of claim 10, wherein in process step a) at least one exogenous nucleic acid sequence encoding an ethylmalonyl-CoA mutase, in particular from at least one bacterium selected from the group consisting of Methylorubrum extorquens, in particular Methylorubrum extorquens TK 0001 DSM 1337, and Rhodobacter sphaeroides, in particular Rhodobacter sphaeroides ATCC 17029, in particular an expression cassette comprising this nucleic acid sequence, is provided, in process step b) the Methylobacteriaceae cell is transformed with the exogenous nucleic acid sequence encoding an ethylmalonyl-CoA mutase, in particular the expression cassette comprising it, and in process step c) a genetically modified Methylobacteriaceae cell having at least one exogenous, glyoxylate reductase, which additionally has at least one exogenous nucleic acid sequence encoding an ethylmalonyl-CoA mutase, is obtained.

12. A genetically modified Methylobacteriaceae cell of claim 1, wherein the cell is present alive or dead or lyophilised or in the form of a cell lysate or cell extract recovered from a genetically modified Methylobacteriaceae cell.

13. A biocatalyst comprising a genetically modified Methylobacteriaceae cell of claim 1, wherein it is arranged on a carrier.

14. A bioreactor comprising a genetically modified Methylobacteriaceae cell of claim 1.

15. A process for producing a product containing glycolic acid from a reactant containing at least one Cx compound, comprising the process steps of:
x) providing a genetically modified Methylobacteriaceae cell of claim 1, a reaction medium and the reactant containing at least one Cx compound,
y) converting the reactant under conditions that enable the formation of glycolic acid from the Cx compound, and
z) obtaining the product, containing glycolic acid, from the reaction medium.

16. The process of claim 15, wherein the Cx compound is a Cx compound with x=1, 2 or 4, in particular formic acid, methanol, methane, methylamine, acetic acid or succinic acid.

17. The process of claim 15, wherein the product containing glycolic acid is a product containing glycolic acid and lactic acid.

18. The process of claim 15, wherein the Cx compound is produced from CO₂, in particular synthesis gas comprising a mixture of CO₂, CO and H₂, in particular by means of a heterogeneous catalytic chemical process.

19. The process of claim 18, wherein the CO₂, in particular synthesis gas, is produced by chemical conversion of organic compounds or materials, in particular of sewage sludge and other biogenic residual and waste materials.

20. A process for producing polyglycolic acid, polylactic acid or polylactide-co-glycolide, comprising carrying out a process of claim 15 and subsequently polymerising the glycolic acid, lactic acid or mixture of glycolic acid and lactic acid obtained from these processes.


SEQUENCE LISTING (13 sequences)

SEQ ID NO: 1 - DNA, 939 bp, Escherichia coli
  source 1..939: mol_type = unassigned DNA
atggatatcatcttttatcacccaacgttcgatacccaatggtggattgaggcactgcgcaaagctattcctcaggcaagagtcagagcatggaaaagcggagataatgactctgctgattatgctttagtctggcatcctcctgttgaaatgctggcagggcgcgatcttaaagcggtgttcgcactcggggccggtgttgattctattttgagcaagctacaggcacaccctgaaatgctgaacccttctgttccactttttcgcctggaagataccggtatgggcgagcaaatgcaggaatatgctgtcagtcaggtgctgcattggtttcgacgttttgacgattatcgcatccagcaaaatagttcgcattggcaaccgctgcctgaatatcatcgggaagattttaccatcggcattttgggcgcaggcgtactgggcagtaaagttgctcagagtctgcaaacctggcgctttccgctgcgttgctggagtcgaacccgtaaatcgtggcctggcgtgcaaagctttgccggacgggaagaactgtctgcatttctgagccaatgtcgggtattgattaatttgttaccgaatacccctgaaaccgtcggcattattaatcaacaattactcgaaaaattaccggatggcgcgtatctcctcaacctggcgcgtggtgttcatgttgtggaagatgacctgctcgcggcgctggatagcggcaaagttaaaggcgcaatgttggatgtttttaatcgtgaacccttaccgcctgaaagtccgctctggcaacatccacgcgtgacgataacaccacatgtcgccgcgattacccgtcccgctgaagctgtggagtacatttctcgcaccattgcccagctcgaaaaaggggagagggtctgcgggcaagtcgaccgcgcacgcggctactaa

SEQ ID NO: 2 - protein, 312 aa, Escherichia coli
MDIIFYHPTFDTQWWIEALRKAIPQARVRAWKSGDNDSADYALVWHPPVEMLAGRDLKAVFALGAGVDSILSKLQAHPEMLNPSVPLFRLEDTGMGEQMQEYAVSQVLHWFRRFDDYRIQQNSSHWQPLPEYHREDFTIGILGAGVLGSKVAQSLQTWRFPLRCWSRTRKSWPGVQSFAGREELSAFLSQCRVLINLLPNTPETVGIINQQLLEKLPDGAYLLNLARGVHVVEDDLLAALDSGKVKGAMLDVFNREPLPPESPLWQHPRVTITPHVAAITRPAEAVEYISRTIAQLEKGERVCGQVDRARGY

SEQ ID NO: 3 - DNA, 939 bp, Escherichia coli
  source 1..939: mol_type = unassigned DNA
atggacatcatcttctaccacccgaccttcgacacccagtggtggatcgaggccctgcgcaaggccatcccgcaggcccgcgtccgcgcctggaagtcgggcgacaacgactcggccgactacgccctggtctggcacccgccggtcgagatgctcgccggccgcgacctcaaggccgtgttcgccctcggcgccggcgtcgactcgatcctctcgaagctgcaggcccacccggagatgctcaacccgtcggtcccgctgttccgcctggaggacaccggcatgggcgagcagatgcaggagtacgccgtgtcgcaggtcctccactggttccgccgcttcgacgactaccgcatccagcagaactcgtcgcactggcagccgctgccggagtaccaccgcgaggacttcaccatcggcatcctgggcgccggcgtgctcggctcgaaggtcgcccagtcgctccagacctggcgcttcccgctccgctgctggtcgcgcacccgcaagtcgtggccgggcgtccagtcgttcgccggccgcgaggagctctcggccttcctctcgcagtgccgcgtcctgatcaacctcctcccgaacaccccggagaccgtgggcatcatcaaccagcagctgctcgaaaagctgccggacggcgcctacctgctcaacctggcccgcggcgtccacgtggtggaggatgacctgctggccgccctcgactcgggcaaggtgaagggcgccatgctcgacgtgttcaaccgcgagccgctgccgccggagtcgccgctctggcagcatccgcgcgtgaccatcaccccgcacgtggccgccatcacccgcccggccgaggccgtcgagtacatctcgcgcaccatcgcccagctggagaagggcgagcgcgtctgcggccaggtcgaccgcgcccgcggctactga

SEQ ID NO: 4 - DNA, 2067 bp, synthetic construct
  source 1..2067: mol_type = other DNA
atgagcgcgcaagcgagcgtcgccgaggtcaagcgcgacaagccgtggatcatccgcacctatgcggggcattccacggcggcggagtccaacaagctctatcgcggcaacctggccaagggtcagaccggcctctcggtcgccttcgatctgccgacgcagaccggctacgacccggatcacgaactcgcccgcggcgaggtcggcaaggtcggcgtctcgatcgcgcatctcggcgacatgcgggccctgttcgaccagatcccgctggcacagatgaacacctcgatgacgatcaacgccacggcgccgtggctgttgtcgctctacctcgcggtggccgaggagcagggcgcaccgctcgcggcactccagggcaccacgcagaacgacatcatcaaggaatatctctcgcgcggcacctacgtgtttccgcccgcgccttcgcttcggctcaccaaggacgtgatcttgttcacgaccaagaacgtgccgaagtggaacccgatgaacgtctgctcctaccacctgcaggaggcgggggcgacgccggttcaggaactctcctacgcgctggccatcgccatcgccgtgctcgacacggtgcgcgacgaccccgatttcgacgaggccagcttctccgacgtcttcagccggatctcgttcttcgtgaacgccggcatgcggttcgtcaccgagatctgcaagatgcgggcgttcgccgagctatgggacgagatcgcccaggagcgctacggcatcaccgacgctaagaagcggattttccgctacggcgtgcaggtcaacagcctcgggctaacggagcagcaacccgagaacaacgtccaccgcatcctaatcgagatgctggcggtgacgctgtcgaaacgcgcccgcgcccgcgcggtgcagctcccggcctggaacgaggcgctcggcctgccgcgcccgtgggaccagcaatggtcgatgcggatgcagcagatcctcgccttcgagaccgacctgctggaatacgacgatatcttcgacggctcgacggtgatcgaggcccgcgtcgaggcgctcaaggagcagacccgggccgagctgacgcgcatcgctgagatcggcggggcggtcacggcggtcgaggcgggcgaactcaagcgggcgctggtcgagtcgaatgcccggcgcatctcggcgatcgagaagggcgagcagatcgtcgtcggcgtcaacaagtggcagcagggcgagccctcgccgctgacggccggagacggcgcgatcttcaccgtctcggagacggtggagatggaggccgagacccgcatccgcgagtggcgctccaagcgcgacgagcgcgcggtcggccaggcgctcgccgatctggaacaggccgcgcgctcgggcgccaacatcatgccgccctccatcgccgctgcgaaagcgggcgtgacgacgggcgaatggggccagcgcctgcgcgaggtgttcggcgagtaccgcgcgcccacgggtgtgacgctgcagaccgtcacttccggcgcggcggaggatgcccgcctgctcatcgccgacctcggcgaacggctcggcgagacgccccgcctcgtggtcggcaagccgggcctcgacggccattccaacggcgccgagcagatcgctttgcgcgcccgcgatgtcggcttcgatgtgacctatgacggcatccgccagacgccgaccgagatcgtcgccaaggcgaaggaacgcggcgcccacgtcatcggcctctcggtgctgtcgggcagccatgtccccctggtgcgcgaggtgaaggccaagctgcgcgaggccgggctcgatcacgtgccggtcgtcgtcggcggcattatctcgaccgaggacgagttggtgctcaagaatatgggcgtcaccgccgtctacacgccgaaggattacgagctcgacaagatcatggtcgggctcgccaaggtggtggagcgggcactcgacaagcgcgctgccgaccgggcggatacggaggccggcgtgccgggcgcgccgaagcggaacgagagcggggcgcaggtcttctga

SEQ ID NO: 5 - protein, 688 aa, synthetic construct
MSAQASVAEVKRDKPWIIRTYAGHSTAAESNKLYRGNLAKGQTGLSVAFDLPTQTGYDPDHELARGEVGKVGVSIAHLGDMRALFDQIPLAQMNTSMTINATAPWLLSLYLAVAEEQGAPLAALQGTTQNDIIKEYLSRGTYVFPPAPSLRLTKDVILFTTKNVPKWNPMNVCSYHLQEAGATPVQELSYALAIAIAVLDTVRDDPDFDEASFSDVFSRISFFVNAGMRFVTEICKMRAFAELWDEIAQERYGITDAKKRIFRYGVQVNSLGLTEQQPENNVHRILIEMLAVTLSKRARARAVQLPAWNEALGLPRPWDQQWSMRMQQILAFETDLLEYDDIFDGSTVIEARVEALKEQTRAELTRIAEIGGAVTAVEAGELKRALVESNARRISAIEKGEQIVVGVNKWQQGEPSPLTAGDGAIFTVSETVEMEAETRIREWRSKRDERAVGQALADLEQAARSGANIMPPSIAAAKAGVTTGEWGQRLREVFGEYRAPTGVTLQTVTSGAAEDARLLIADLGERLGETPRLVVGKPGLDGHSNGAEQIALRARDVGFDVTYDGIRQTPTEIVAKAKERGAHVIGLSVLSGSHVPLVREVKAKLREAGLDHVPVVVGGIISTEDELVLKNMGVTAVYTPKDYELDKIMVGLAKVVERALDKRAADRADTEAGVPGAPKRNESGAQVF

SEQ ID NO: 6 - DNA, 1959 bp, Rhodobacter sphaeroides
  source 1..1959: mol_type = unassigned DNA
atgacccagaaggatagcccctggctgttccgcacctatgccggccattccacggcgaaggcctcgaatgcgctctatcgcaccaacctcgccaagggacagaccgggctctctgtcgccttcgacctgccgacccagaccggctacgacagcgacgatgcgctggcgcggggcgaggtgggcaaggtcggcgtgccgatctgccatctgggcgacatgcggatgctgttcgaccagatcccgctcgagcaaatgaacacctcgatgacgatcaacgcgaccgctccctggctgctcgcgctctatatcgcggtggccgaggagcagggcgccgacatctcgaagctgcagggcacggtgcagaacgatctgatgaaggagtatctcagccgcggcacctacatctgcccgccgcggccctcgctgcgcatgatcaccgacgtggcggcctatacgcgggtccatctgccgaagtggaacccgatgaacgtctgctcctaccatctgcaggaggccggggccacgcccgagcaggagctggccttcgcgctcgcgaccggcatcgcggtgctcgacgacctgcgcactaaggtgccggccgagcatttcccggccatggtcgggcgcatctcgttcttcgtgaatgcgggcatccgcttcgtgaccgagatgtgcaagatgcgcgccttcgtcgatctctgggacgagatctgccgcgaccgctacggcatcgaggaagagaaataccgccgcttccgctacggggtgcaggtcaactcgctgggcctgaccgagcagcagcccgagaacaacgtctaccggatcctgatcgagatgctggccgtgacgctctcgaagaaggcccgcgcccgggcggtgcagctgcccgcctggaacgaggcgctggggctgccgcggccctgggaccagcaatggtcgctccggatgcagcagatcctggcctacgaatcggacctgctggaatatgaggatctgttcgacggcaaccccgccatcgagcgcaaggtcgaggcgctgaaggacggggcgcgggaagagctggcccatatcgaggcgatgggcggcgccatcgaggccatcgactacatgaaggcgcggctcgtcgagtcgaacgccgagcgcatcgcccgggtcgagaccggcgagacggtggtggtcggcgtgaaccgctggacctccggcgcgccctcgccgctcaccacgggcgacggcgcgatcatggtggccgaccccgaggccgagcgcgaccagatcgcccggctcgaagcctggcgcgcgggccgcgacggggcggccgtcgcagcagcgctggccgagctgcgccgtgccgccacctcgggcgagaatgtgatgccggcctcgatcgccgccgcgaaggccggggccacgaccggcgaatgggcggccgagctgcgccgcgcgttcggcgagttccgcggccccaccggagtggcgcgcgcgccctcgaaccgcaccgacgggctcgatccgatccgcgaggcggtgcaggcggtctcggcgcggctcgggcggccgctcaagttcgtcgtgggcaagcccggcctcgacggccattccaacggcgcggagcagatcgccgcccgcgcccgcgactgcggcatggacatcacctacgacgggatccgcctgaccccggccgagatcgtggccaaggccgccgacgagcgggcccatgtgctgggcctctcgatcctgtcgggctcgcacatgccgctcgtgaccgaggtgctggccgagatgcggcgcgcggggctcgacatgccgctgatcgtgggcggcatcatccccgaggaggatgccgccgagctgcgggcctcgggcgtggccgcggtctatacgcccaaggatttcgagctgaaccggatcatgatggatatcgtgggtctggtggaccgcaccgccctcgcggcggaatag

SEQ ID NO: 7 - protein, 652 aa, Rhodobacter sphaeroides
MTQKDSPWLFRTYAGHSTAKASNALYRTNLAKGQTGLSVAFDLPTQTGYDSDDALARGEVGKVGVPICHLGDMRMLFDQIPLEQMNTSMTINATAPWLLALYIAVAEEQGADISKLQGTVQNDLMKEYLSRGTYICPPRPSLRMITDVAAYTRVHLPKWNPMNVCSYHLQEAGATPEQELAFALATGIAVLDDLRTKVPAEHFPAMVGRISFFVNAGIRFVTEMCKMRAFVDLWDEICRDRYGIEEEKYRRFRYGVQVNSLGLTEQQPENNVYRILIEMLAVTLSKKARARAVQLPAWNEALGLPRPWDQQWSLRMQQILAYESDLLEYEDLFDGNPAIERKVEALKDGAREELAHIEAMGGAIEAIDYMKARLVESNAERIARVETGETVVVGVNRWTSGAPSPLTTGDGAIMVADPEAERDQIARLEAWRAGRDGAAVAAALAELRRAATSGENVMPASIAAAKAGATTGEWAAELRRAFGEFRGPTGVARAPSNRTDGLDPIREAVQAVSARLGRPLKFVVGKPGLDGHSNGAEQIAARARDCGMDITYDGIRLTPAEIVAKAADERAHVLGLSILSGSHMPLVTEVLAEMRRAGLDMPLIVGGIIPEEDAAELRASGVAAVYTPKDFELNRIMMDIVGLVDRTALAAE

SEQ ID NO: 8 - DNA, 1959 bp, Rhodobacter sphaeroides
  source 1..1959: mol_type = unassigned DNA
atgacccagaaggactcgccgtggctgttccgcacctacgccggccactcgaccgccaaggcctcgaacgccctctaccgcaccaacctcgccaagggccagaccggcctctcggtcgccttcgacctcccgacccagaccggctacgactcggacgacgccctggcccgcggcgaggtcggcaaggtcggcgtcccgatctgccacctcggcgacatgcgcatgctcttcgaccagatcccgctggagcagatgaacacctcgatgaccatcaacgccaccgccccgtggctcctggccctctacatcgccgtggccgaggagcagggcgccgatatctcgaagctgcagggcaccgtgcagaacgacctcatgaaggagtacctctcgcgcggcacctacatctgcccgccgcgcccgtcgctgcgcatgatcaccgacgtcgccgcctacacccgcgtccacctcccgaagtggaacccgatgaacgtctgctcgtaccacctccaggaggccggcgccaccccggagcaggagctcgccttcgccctggccaccggcatcgccgtcctggatgacctgcgcaccaaggtcccggccgagcacttcccggcgatggttggccgcatctcgttcttcgtgaacgccggcatccgcttcgtcaccgagatgtgcaagatgcgcgccttcgtggacctctgggacgagatctgccgcgaccgctacggcatcgaggaggagaagtaccgccgcttccgctacggcgtgcaggtcaactcgctgggcctcaccgagcagcagccggagaacaacgtgtaccgcatcctgatcgagatgctcgccgtcaccctgtcgaagaaggcccgcgcccgcgccgtccagctcccggcctggaacgaggccctcggcctcccgcgcccgtgggaccagcagtggtcgctccgcatgcagcagatcctcgcctacgagtcggacctcctcgaatacgaggacctcttcgacggcaacccggccatcgagcgcaaggtcgaggccctgaaggacggcgcccgcgaggagctcgcccatatcgaggcgatgggtggcgccatcgaggccatcgactacatgaaggcccgcctcgtggagtcgaacgccgagcgcatcgcccgcgtggagaccggcgagaccgtggtcgtcggcgtcaaccgctggacctcgggcgccccgtcgccgctcaccaccggcgacggcgccatcatggtcgccgacccggaggccgagcgcgatcagatcgcccgcctcgaagcctggcgcgccggccgcgacggcgccgccgtcgccgccgccctcgccgagctccgccgcgccgccacctcgggcgagaacgtcatgccggcctcgatcgccgccgccaaggccggcgccacgaccggcgagtgggccgccgagctccggcgcgccttcggcgagttccgcggcccgaccggcgtcgcccgcgccccgtcgaaccgcaccgacggcctcgatccgatccgcgaggccgtgcaggccgtctcggcccgcctcggccgcccgctcaagttcgtcgtcggcaagccgggcctggacggccattcgaacggcgccgagcagatcgccgcccgcgcccgggactgcggcatggacatcacctacgacggcatccgcctcaccccggccgagatcgtcgccaaggccgccgatgagcgcgcccatgtgctgggcctctcgatcctgtcgggctcgcacatgccgctggtcaccgaggtcctggccgagatgcgccgcgccggcctcgacatgccgctcatcgtcggcggcatcatcccggaggaggacgccgccgagctgcgcgcctcgggcgtcgccgccgtctacaccccgaaggacttcgagctgaaccgcatcatgatggacatcgtgggcctggtggaccgcaccgccctcgccgccgagtga

SEQ ID NO: 9 - DNA, 8412 bp, unidentified
  source 1..8412: mol_type = unassigned DNA
ggccgcgcgaattcgagctcggtactgacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcagttaccatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagaaaagataaattatctctggcggtgttgacatgtgagcggataacaatgatactgagcacatcgagagggacacggcgactctagaattaaagaggagaaattaaccatggcgagctggagccatccgcagttcgaaaagatcgaaggccgccatatgtcgggcggatcctgaactagtcctgcaggtaccgtaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctaggagcttgcggccctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcgaggctccgcgccctcgatccgaccgccccccacaaggctggggcggatcgggggcgcacggcctgaagggtgcaggggaaccctgccgcacgcaaactcgcagagtttgcatagtgtgcgccattcagataccttcatggcaggccccaccgtggattttgctgtcatggttcgattcatcacgacgcccgcaacgccgactgcacagcggcccgcaccacgaacgcctgcgcagcaggaagctgcggctgaactgaaggctgcgcgaactgccgaagcagcccgccgcctcgacacccggcgcaaggtggtgctcgggggcgcgctcctggcactggcagaacgtgatgcagcagcagccgcgatgctggcccggctgaagcagatgctgacccgcccagcagatcggaagctgttcgcggatctacccgatggctgacggtgcaatcttccacctcgatgtgcgcacggtcagccggtcagatggccgctcggctgtcgccgctgccgcctatcggtcggccagccgtctgcacgatgcacgcaccggcctgacccacgacttcgcgcgcaagcggggcgtcttgacaaccttcatcgccgccccggacggctgcgactggatcacagacagggacacgctctggaacgcggctgaggcggcagagaagcgcaagaacagcaccgtggcccgtgagtggctggtggccctccctgacgctctggatgctacacagcgggccgacctggcgcgggcgctggcggtcgaactggcgcagcggttcggggtggccgtggatgtggcaatccacgcagcttcgggcgagggcgaccagcggaaccatcacgcgcatttgctgaccacgacgcgcaccgcaggcccggacgggctgggcgacaagacccgcgttctggatgcagccaaaacggggggcgcagaaatctacgccatgcgggcttggtgggcgggcacggtcaacgatgcactcgcggccgcacaaagctcggcccgcgtcgatcaccgccgcatagccgtgatcgccgccgatgcgcgggccgaggccgaggcgctggagaaacaggccaaagccgtcgaaaccctgaacgccaagcccgccgacgccggggggctgtggaagggccttggcagcgccgcccgcgcaatatgggaaggcggcatgaccgccctcaaatcgaccgccgagacagccaacgagctgcgggagaaggccaacgagctgcgacagaaggcagcccgctacacccgcgccaccgaacctcaccacggccccgtcctgacggccttcttgcggcgcatggctcccgtctgggcggagcaggccaaggccgaggaagcccggagagccgcgcaggaggccgcacagcgcgcactgatggccgagagggctgaacaggcccggaaggccaaggaagcctctgaggccgctcgccgggccgctacggcagcgcgagaggccgaggaagcccgcgaacgctgcgcccgcgaggtgttgccgctcatccgccgcgcccggcacgacccgatcaccgccgaagtgatcaagcgggagggcatcgacctcgaccgccccgaccaacaggtggcccgcgacccgatctggctcatggccggacgccaagatcaggcgcacccggtctggctgatcgtccacgcctacaccgccgcgcgcgacaggcacgcgaacctgctggcgagaatcgaagccaaaaccgcagcccagatcgccgctgccgccaaaccaaagcccaagcctgccagtcccgccccgaccgaacccgaacccgatccagacccaccgccccggcgggattggggcgggccgtctggaccctgatccaccccccattctcagcaactccgagccagccagaggcgctggctcggggcatctggacccccttcctcaccccaaattttcacgaaaaaggggcgcagcccttcttgttcaataagttctataagttcaggcgaaaatcgcctttcattttcaatggcttgcgcgcctatcggggagaaatggggccgcaaaagggagaaatggggccgcaaaagggagaaatggggccgatagcggctgttgacagggggatattttgccccctacatttctccccatgacaaagaccacgcaaatcgccgctgatcgagcctatgacgaggccaaaaccgtgctgcccgcagaggtggcgcggggggtctacatgcgcaacgcccccagcctcgccgccctcaagctgatgcacctgctgatcggtaaggcgggcgggcgtatggccgaacccattcggcacgaaatccgcctctcagacatccgcaagatcgacgggatgaagaaccatgacagggccagcctcaccccccttttcgcggaattgtcggcggctgtcctgacccatgatgaccccgaaaaaatggtcgtgaccattggcggcttgctggacttcgcccgcatcgactaccgccacgaagtcagcaacgatctgctggtgtcgtggtattttggcggggccttccgcgagatggccgagaagtcctgccactgggcgatcctcgaccggcagaccgtttttgccctgtccagcaagtattcgatcctgctgttccagcacatcgccagcctcgtgaacctcgatcacgtcgccagcaagaccttcaccgtggccgaactgcgggccgtgctaggggtgccgcacggaaagatggagcggtggaacgacgtgaaccgctttgcgctgacgcccgcgattgccgagatcaaccagacctcgcgcctgatcctgaccgcgaccccgaacaagatcgggcggacggtcgccagcgtgacaatcacgtgggcggagaagcctcccgagggcaagcaggaagccaagcgcgaactggaccgccccaaggtgggccgagaggcgcggcgcaacggctcagccgagaccgtggcgcgggccttcccgagctcgggcagcatcgagttcgatgaccactggaaggccctcaagcgggcggcgggctgcaacatggacaacaccatgattgccgagaagttccgggcgtggtgcgccagcaaggggctttccctcgacggccgaaacatcgagcaggcgttcagcaacttctgcgccacagtgggcaaggtctgaggccgcgcccgctgccagtgacccgccgccgccgaccgtgagcgggccgcagcccttccagcgccgcccaagagggcggcaagccccggtctgccccctcgtcgcccgcaaagcgggcagggggcagaccggggcgctggaacctttgggctgctttgacggccagacagcccccgccgcgcaagcgcgccattcccccacccgtggccgggcgggggaatggtaaagcatgtgggggcagtccagccgtcaatccgcgctccgcaccacggcgcctcgagcaacaccttcttcacgaggcagacctctcgacggagttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagctcgcacattcagcagcgtttttcagcgcgttttcgatcaacgtttcaatgttggtatcaacaccaggtttaactttgaacttatcggcactgacggttactgattttgaacttttgctttgccacggaacggtctgcgttgtcgggaagatgcgtgatctgatccttcaactcagcaaaagttcgccaatacgcaaaccgcctctccccgcgcgttggccgattcattaatgcag

SEQ ID NO: 10 - DNA, 9352 bp, unidentified
  source 1..9352: mol_type = unassigned DNA
ggccgcgcgaattcgagctcggtactgacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcagttaccatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagaaaagataaattatctctggcggtgttgacatgtgagcggataacaatgatactgagcacatcgagagggacacggcgactctagaattaaagaggagaaattaaccatggacatcatcttctaccacccgaccttcgacacccagtggtggatcgaggccctgcgcaaggccatcccgcaggcccgcgtccgcgcctggaagtcgggcgacaacgactcggccgactacgccctggtctggcacccgccggtcgagatgctcgccggccgcgacctcaaggccgtgttcgccctcggcgccggcgtcgactcgatcctctcgaagctgcaggcccacccggagatgctcaacccgtcggtcccgctgttccgcctggaggacaccggcatgggcgagcagatgcaggagtacgccgtgtcgcaggtcctccactggttccgccgcttcgacgactaccgcatccagcagaactcgtcgcactggcagccgctgccggagtaccaccgcgaggacttcaccatcggcatcctgggcgccggcgtgctcggctcgaaggtcgcccagtcgctccagacctggcgcttcccgctccgctgctggtcgcgcacccgcaagtcgtggccgggcgtccagtcgttcgccggccgcgaggagctctcggccttcctctcgcagtgccgcgtcctgatcaacctcctcccgaacaccccggagaccgtgggcatcatcaaccagcagctgctcgaaaagctgccggacggcgcctacctgctcaacctggcccgcggcgtccacgtggtggaggatgacctgctggccgccctcgactcgggcaaggtgaagggcgccatgctcgacgtgttcaaccgcgagccgctgccgccggagtcgccgctctggcagcatccgcgcgtgaccatcaccccgcacgtggccgccatcacccgcccggccgaggccgtcgagtacatctcgcgcaccatcgcccagctggagaagggcgagcgcgtctgcggccaggtcgaccgcgcccgcggctactgacatggcgagctggagccatccgcagttcgaaaagatcgaaggccgccatatgtcgggcggatcctgaactagtcctgcaggtaccgtaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctaggagcttgcggccctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcgaggctccgcgccctcgatccgaccgccccccacaaggctggggcggatcgggggcgcacggcctgaagggtgcaggggaaccctgccgcacgcaaactcgcagagtttgcatagtgtgcgccattcagataccttcatggcaggccccaccgtggattttgctgtcatggttcgattcatcacgacgcccgcaacgccgactgcacagcggcccgcaccacgaacgcctgcgcagcaggaagctgcggctgaactgaaggctgcgcgaactgccgaagcagcccgccgcctcgacacccggcgcaaggtggtgctcgggggcgcgctcctggcactggcagaacgtgatgcagcagcagccgcgatgctggcccggctgaagcagatgctgacccgcccagcagatcggaagctgttcgcggatctacccgatggctgacggtgcaatcttccacctcgatgtgcgcacggtcagccggtcagatggccgctcggctgtcgccgctgccgcctatcggtcggccagccgtctgcacgatgcacgcaccggcctgacccacgacttcgcgcgcaagcggggcgtcttgacaaccttcatcgccgccccggacggctgcgactggatcacagacagggacacgctctggaacgcggctgaggcggcagagaagcgcaagaacagcaccgtggcccgtgagtggctggtggccctccctgacgctctggatgctacacagcgggccgacctggcgcgggcgctggcggtcgaactggcgcagcggttcggggtggccgtggatgtggcaatccacgcagcttcgggcgagggcgaccagcggaaccatcacgcgcatttgctgaccacgacgcgcaccgcaggcccggacgggctgggcgacaagacccgcgttctggatgcagccaaaacggggggcgcagaaatctacgccatgcgggcttggtgggcgggcacggtcaacgatgcactcgcggccgcacaaagctcggcccgcgtcgatcaccgccgcatagccgtgatcgccgccgatgcgcgggccgaggccgaggcgctggagaaacaggccaaagccgtcgaaaccctgaacgccaagcccgccgacgccggggggctgtggaagggccttggcagcgccgcccgcgcaatatgggaaggcggcatgaccgccctcaaatcgaccgccgagacagccaacgagctgcgggagaaggccaacgagctgcgacagaaggcagcccgctacacccgcgccaccgaacctcaccacggccccgtcctgacggccttcttgcggcgcatggctcccgtctgggcggagcaggccaaggccgaggaagcccggagagccgcgcaggaggccgcacagcgcgcactgatggccgagagggctgaacaggcccggaaggccaaggaagcctctgaggccgctcgccgggccgctacggcagcgcgagaggccgaggaagcccgcgaacgctgcgcccgcgaggtgttgccgctcatccgccgcgcccggcacgacccgatcaccgccgaagtgatcaagcgggagggcatcgacctcgaccgccccgaccaacaggtggcccgcgacccgatctggctcatggccggacgccaagatcaggcgcacccggtctggctgatcgtccacgcctacaccgccgcgcgcgacaggcacgcgaacctgctggcgagaatcgaagccaaaaccgcagcccagatcgccgctgccgccaaaccaaagcccaagcctgccagtcccgccccgaccgaacccgaacccgatccagacccaccgccccggcgggattggggcgggccgtctggaccctgatccaccccccattctcagcaactccgagccagccagaggcgctggctcggggcatctggacccccttcctcaccccaaattttcacgaaaaaggggcgcagcccttcttgttcaataagttctataagttcaggcgaaaatcgcctttcattttcaatggcttgcgcgcctatcggggagaaatggggccgcaaaagggagaaatggggccgcaaaagggagaaatggggccgatagcggctgttgacagggggatattttgccccctacatttctccccatgacaaagaccacgcaaatcgccgctgatcgagcctatgacgaggccaaaaccgtgctgcccgcagaggtggcgcggggggtctacatgcgcaacgcccccagcctcgccgccctcaagctgatgcacctgctgatcggtaaggcgggcgggcgtatggccgaacccattcggcacgaaatccgcctctcagacatccgcaagatcgacgggatgaagaaccatgacagggccagcctcaccccccttttcgcggaattgtcggcggctgtcctgacccatgatgaccccgaaaaaatggtcgtgaccattggcggcttgctggacttcgcccgcatcgactaccgccacgaagtcagcaacgatctgctggtgtcgtggtattttggcggggccttccgcgagatggccgagaagtcctgccactgggcgatcctcgaccggcagaccgtttttgccctgtccagcaagtattcgatcctgctgttccagcacatcgccagcctcgtgaacctcgatcacgtcgccagcaagaccttcaccgtggccgaactgcgggccgtgctaggggtgccgcacggaaagatggagcggtggaacgacgtgaaccgctttgcgctgacgcccgcgattgccgagatcaaccagacctcgcgcctgatcctgaccgcgaccccgaacaagatcgggcggacggtcgccagcgtgacaatcacgtgggcggagaagcctcccgagggcaagcaggaagccaagcgcgaactggaccgccccaaggtgggccgagaggcgcggcgcaacggctcagccgagaccgtggcgcgggccttcccgagctcgggcagcatcgagttcgatgaccactggaaggccctcaagcgggcggcgggctgcaacatggacaacaccatgattgccgagaagttccgggcgtggtgcgccagcaaggggctttccctcgacggccgaaacatcgagcaggcgttcagcaacttctgcgccacagtgggcaaggtctgaggccgcgcccgctgccagtgacccgccgccgccgaccgtgagcgggccgcagcccttccagcgccgcccaagagggcggcaagccccggtctgccccctcgtcgcccgcaaagcgggcagggggcagaccggggcgctggaacctttgggctgctttgacggccagacagcccccgccgcgcaagcgcgccattcccccacccgtggccgggcgggggaatggtaaagcatgtgggggcagtccagccgtcaatccgcgctccgcaccacggcgcctcgagcaacaccttcttcacgaggcagacctctcgacggagttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagctcgcacattcagcagcgtttttcagcgcgttttcgatcaacgtttcaatgttggtatcaacaccaggtttaactttgaacttatcggcactgacggttactgattttgaacttttgctttgccacggaacggtctgcgttgtcgggaagatgcgtgatctgatccttcaactcagcaaaagttcgccaatacgcaaaccgcctctccccgcgcgttggccgattcattaatgcag

SEQ ID NO: 11 - DNA, 11437 bp, unidentified
  source 1..11437: mol_type = unassigned DNA
ggccgcgcgaattcgagctcggtactgacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcagttaccatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagaaaagataaattatctctggcggtgttgacatgtgagcggataacaatgatactgagcacatcgagagggacacggcgactctagaattaaagaggagaaattaaccatggacatcatcttctaccacccgaccttcgacacccagtggtggatcgaggccctgcgcaaggccatcccgcaggcccgcgtccgcgcctggaagtcgggcgacaacgactcggccgactacgccctggtctggcacccgccggtcgagatgctcgccggccgcgacctcaaggccgtgttcgccctcggcgccggcgtcgactcgatcctctcgaagctgcaggcccacccggagatgctcaacccgtcggtcccgctgttccgcctggaggacaccggcatgggcgagcagatgcaggagtacgccgtgtcgcaggtcctccactggttccgccgcttcgacgactaccgcatccagcagaactcgtcgcactggcagccgctgccggagtaccaccgcgaggacttcaccatcggcatcctgggcgccggcgtgctcggctcgaaggtcgcccagtcgctccagacctggcgcttcccgctccgctgctggtcgcgcacccgcaagtcgtggccgggcgtccagtcgttcgccggccgcgaggagctctcggccttcctctcgcagtgccgcgtcctgatcaacctcctcccgaacaccccggagaccgtgggcatcatcaaccagcagctgctcgaaaagctgccggacggcgcctacctgctcaacctggcccgcggcgtccacgtggtggaggatgacctgctggccgccctcgactcgggcaaggtgaagggcgccatgctcgacgtgttcaaccgcgagccgctgccgccggagtcgccgctctggcagcatccgcgcgtgaccatcaccccgcacgtggccgccatcacccgcccggccgaggccgtcgagtacatctcgcgcaccatcgcccagctggagaagggcgagcgcgtctgcggccaggtcgaccgcgcccgcggctactgacatggcgagctggagccatccgcagttcgaaaagatcgaaggccgccatatgtcgggcgaaagaggagaaattaaccatgagcgcgcaagcgagcgtcgccgaggtcaagcgcgacaagccgtggatcatccgcacctatgcggggcattccacggcggcggagtccaacaagctctatcgcggcaacctggccaagggtcagaccggcctctcggtcgccttcgatctgccgacgcagaccggctacgacccggatcacgaactcgcccgcggcgaggtcggcaaggtcggcgtctcgatcgcgcatctcggcgacatgcgggccctgttcgaccagatcccgctggcacagatgaacacctcgatgacgatcaacgccacggcgccgtggctgttgtcgctctacctcgcggtggccgaggagcagggcgcaccgctcgcggcactccagggcaccacgcagaacgacatcatcaaggaatatctctcgcgcggcacctacgtgtttccgcccgcgccttcgcttcggctcaccaaggacgtgatcttgttcacgaccaagaacgtgccgaagtggaacccgatgaacgtctgctcctaccacctgcaggaggcgggggcgacgccggttcaggaactctcctacgcgctggccatcgccatcgccgtgctcgacacggtgcgcgacgaccccgatttcgacgaggccagcttctccgacgtcttcagccggatctcgttcttcgtgaacgccggcatgcggttcgtcaccgagatctgcaagatgcgggcgttcgccgagctatgggacgagatcgcccaggagcgctacggcatcaccgacgctaagaagcggattttccgctacggcgtgcaggtcaacagcctcgggctaacggagcagcaacccgagaacaacgtccaccgcatcctaatcgagatgctggcggtgacgctgtcgaaacgcgcccgcgcccgcgcggtgcagctcccggcctggaacgaggcgctcggcctgccgcgcccgtgggaccagcaatggtcgatgcggatgcagcagatcctcgccttcgagaccgacctgctggaatacgacgatatcttcgacggctcgacggtgatcgaggcccgcgtcgaggcgctcaaggagcagacccgggccgagctgacgcgcatcgctgagatcggcggggcggtcacggcggtcgaggcgggcgaactcaagcgggcgctggtcgagtcgaatgcccggcgcatctcggcgatcgagaagggcgagcagatcgtcgtcggcgtcaacaagtggcagcagggcgagccctcgccgctgacggccggagacggcgcgatcttcaccgtctcggagacggtggagatggaggccgagacccgcatccgcgagtggcgctccaagcgcgacgagcgcgcggtcggccaggcgctcgccgatctggaacaggccgcgcgctcgggcgccaacatcatgccgccctccatcgccgctgcgaaagcgggcgtgacgacgggcgaatggggccagcgcctgcgcgaggtgttcggcgagtaccgcgcgcccacgggtgtgacgctgcagaccgtcacttccggcgcggcggaggatgcccgcctgctcatcgccgacctcggcgaacggctcggcgagacgccccgcctcgtggtcggcaagccgggcctcgacggccattccaacggcgccgagcagatcgctttgcgcgcccgcgatgtcggcttcgatgtgacctatgacggcatccgccagacgccgaccgagatcgtcgccaaggcgaaggaacgcggcgcccacgtcatcggcctctcggtgctgtcgggcagccatgtccccctggtgcgcgaggtgaaggccaagctgcgcgaggccgggctcgatcacgtgccggtcgtcgtcggcggcattatctcgaccgaggacgagttggtgctcaagaatatgggcgtcaccgccgtctacacgccgaaggattacgagctcgacaagatcatggtcgggctcgccaaggtggtggagcgggcactcgacaagcgcgctgccgaccgggcggatacggaggccggcgtgccgggcgcgccgaagcggaacgagagcggggcgcaggtcttctgagatcctgaactagtcctgcaggtaccgtaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctaggagcttgcggccctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcgaggctccgcgccctcgatccgaccgccccccacaaggctggggcggatcgggggcgcacggcctgaagggtgcaggggaaccctgccgcacgcaaactcgcagagtttgcatagtgtgcgccattcagataccttcatggcaggccccaccgtggattttgctgtcatggttcgattcatcacgacgcccgcaacgccgactgcacagcggcccgcaccacgaacgcctgcgcagcaggaagctgcggctgaactgaaggctgcgcgaactgccgaagcagcccgccgcctcgacacccggcgcaaggtggtgctcgggggcgcgctcctggcactggcagaacgtgatgcagcagcagccgcgatgctggcccggctgaagcagatgctgacccgcccagcagatcggaagctgttcgcggatctacccgatggctgacggtgcaatcttccacctcgatgtgcgcacggtcagccggtcagatggccgctcggctgtcgccgctgccgcctatcggtcggccagccgtctgcacgatgcacgcaccggcctgacccacgacttcgcgcgcaagcggggcgtcttgacaaccttcatcgccgccccggacggctgcgactggatcacagacagggacacgctctggaacgcggctgaggcggcagagaagcgcaagaacagcaccgtggcccgtgagtggctggtggccctccctgacgctctggatgctacacagcgggccgacctggcgcgggcgctggcggtcgaactggcgcagcggttcggggtggccgtggatgtggcaatccacgcagcttcgggcgagggcgaccagcggaaccatcacgcgcatttgctgaccacgacgcgcaccgcaggcccggacgggctgggcgacaagacccgcgttctggatgcagccaaaacggggggcgcagaaatctacgccatgcgggcttggtgggcgggcacggtcaacgatgcactcgcggccgcacaaagctcggcccgcgtcgatcaccgccgcatagccgtgatcgccgccgatgcgcgggccgaggccgaggcgctggagaaacaggccaaagccgtcgaaaccctgaacgccaagcccgccgacgccggggggctgtggaagggccttggcagcgccgcccgcgcaatatgggaaggcggcatgaccgccctcaaatcgaccgccgagacagccaacgagctgcgggagaaggccaacgagctgcgacagaaggcagcccgctacacccgcgccaccgaacctcaccacggccccgtcctgacggccttcttgcggcgcatggctcccgtctgggcggagcaggccaaggccgaggaagcccggagagccgcgcaggaggccgcacagcgcgcactgatggccgagagggctgaacaggcccggaaggccaaggaagcctctgaggccgctcgccgggccgctacggcagcgcgagaggccgaggaagcccgcgaacgctgcgcccgcgaggtgttgccgctcatccgccgcgcccggcacgacccgatcaccgccgaagtgatcaagcgggagggcatcgacctcgaccgccccgaccaacaggtggcccgcgacccgatctggctcatggccggacgccaagatcaggcgcacccggtctggctgatcgtccacgcctacaccgccgcgcgcgacaggcacgcgaacctgctggcgagaatcgaagccaaaaccgcagcccagatcgccgctgccgccaaaccaaagcccaagcctgccagtcccgccccgaccgaacccgaacccgatccagacccaccgccccggcgggattggggcgggccgtctggaccctgatccaccccccattctcagcaactccgagccagccagaggcgctggctcggggcatctggacccccttcctcaccccaaattttcacgaaaaaggggcgcagcccttcttgttcaataagttctataagttcaggcgaaaatcgcctttcattttcaatggcttgcgcgcctatcggggagaaatggggccgcaaaagggagaaatggggccgcaaaagggagaaatggggccgatagcggctgttgacagggggatattttgccccctacatttctccccatgacaaagaccacgcaaatcgccgctgatcgagcctatgacgaggccaaaaccgtgctgcccgcagaggtggcgcggggggtctacatgcgcaacgcccccagcctcgccgccctcaagctgatgcacctgctgatcggtaaggcgggcgggcgtatggccgaacccattcggcacgaaatccgcctctcagacatccgcaagatcgacgggatgaagaaccatgacagggccagcctcaccccccttttcgcggaattgtcggcggctgtcctgacccatgatgaccccgaaaaaatggtcgtgaccattggcggcttgctggacttcgcccgcatcgactaccgccacgaagtcagcaacgatctgctggtgtcgtggtattttggcggggccttccgcgagatggccgagaagtcctgccactgggcgatcctcgaccggcagaccgtttttgccctgtccagcaagtattcgatcctgctgttccagcacatcgccagcctcgtgaacctcgatcacgtcgccagcaagaccttcaccgtggccgaactgcgggccgtgctaggggtgccgcacggaaagatggagcggtggaacgacgtgaaccgctttgcgctgacgcccgcgattgccgagatcaaccagacctcgcgcctgatcctgaccgcgaccccgaacaagatcgggcggacggtcgccagcgtgacaatcacgtgggcggagaagcctcccgagggcaagcaggaagccaagcgcgaactggaccgccccaaggtgggccgagaggcgcggcgcaacggctcagccgagaccgtggcgcgggccttcccgagctcgggcagcatcgagttcgatgaccactggaaggccctcaagcgggcggcgggctgcaacatggacaacaccatgattgccgagaagttccgggcgtggtgcgccagcaaggggctttccctcgacggccgaaacatcgagcaggcgttcagcaacttctgcgccacagtgggcaaggtctgaggccgcgcccgctgccagtgacccgccgccgccgaccgtgagcgggccgcagcccttccagcgccgcccaagagggcggcaagccccggtctgccccctcgtcgcccgcaaagcgggcagggggcagaccggggcgctggaacctttgggctgctttgacggccagacagcccccgccgcgcaagcgcgccattcccccacccgtggccgggcgggggaatggtaaagcatgtgggggcagtccagccgtcaatccgcgctccgcaccacggcgcctcgagcaacaccttcttcacgaggcagacctctcgacggagttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagctcgcacattcagcagcgtttttcagcgcgttttcgatcaacgtttcaatgttggtatcaacaccaggtttaactttgaacttatcggcactgacggttactgattttgaacttttgctttgccacggaacggtctgcgttgtcgggaagatgcgtgatctgatccttcaactcagcaaaagttcgccaatacgcaaaccgcctctccccgcgcgttggccgattcattaatgcag

SEQ ID NO: 12 - DNA, 11329 bp, unidentified
  source 1..11329: mol_type = unassigned DNA
ggccgcgcgaattcgagctcggtactgacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcagttaccatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcacctcgagaaaagataaattatctctggcggtgttgacatgtgagcggataacaatgatactgagcacatcgagagggacacggcgactctagaattaaagaggagaaattaaccatggacatcatcttctaccacccgaccttcgacacccagtggtggatcgaggccctgcgcaaggccatcccgcaggcccgcgtccgcgcctggaagtcgggcgacaacgactcggccgactacgccctggtctggcacccgccggtcgagatgctcgccggccgcgacctcaaggccgtgttcgccctcggcgccggcgtcgactcgatcctctcgaagctgcaggcccacccggagatgctcaacccgtcggtcccgctgttccgcctggaggacaccggcatgggcgagcagatgcaggagtacgccgtgtcgcaggtcctccactggttccgccgcttcgacgactaccgcatccagcagaactcgtcgcactggcagccgctgccggagtaccaccgcgaggacttcaccatcggcatcctgggcgccggcgtgctcggctcgaaggtcgcccagtcgctccagacctggcgcttcccgctccgctgctggtcgcgcacccgcaagtcgtggccgggcgtccagtcgttcgccggccgcgaggagctctcggccttcctctcgcagtgccgcgtcctgatcaacctcctcccgaacaccccggagaccgtgggcatcatcaaccagcagctgctcgaaaagctgccggacggcgcctacctgctcaacctggcccgcggcgtccacgtggtggaggatgacctgctggccgccctcgactcgggcaaggtgaagggcgccatgctcgacgtgttcaaccgcgagccgctgccgccggagtcgccgctctggcagcatccgcgcgtgaccatcaccccgcacgtggccgccatcacccgcccggccgaggccgtcgagtacatctcgcgcaccatcgcccagctggagaagggcgagcgcgtctgcggccaggtcgaccgcgcccgcggctactgacatggcgagctggagccatccgcagttcgaaaagatcgaaggccgccatatgtcgggcgaaagaggagaaattaaccatgacccagaaggactcgccgtggctgttccgcacctacgccggccactcgaccgccaaggcctcgaacgccctctaccgcaccaacctcgccaagggccagaccggcctctcggtcgccttcgacctcccgacccagaccggctacgactcggacgacgccctggcccgcggcgaggtcggcaaggtcggcgtcccgatctgccacctcggcgacatgcgcatgctcttcgaccagatcccgctggagcagatgaacacctcgatgaccatcaacgccaccgccccgtggctcctggccctctacatcgccgtggccgaggagcagggcgccgatatctcgaagctgcagggcaccgtgcagaacgacctcatgaaggagtacctctcgcgcggcacctacatctgcccgccgcgcccgtcgctgcgcatgatcaccgacgtcgccgcctacacccgcgtccacctcccgaagtggaacccgatgaacgtctgctcgtaccacctccaggaggccggcgccaccccggagcaggagctcgccttcgccctggccaccggcatcgccgtcctggatgacctgcgcaccaaggtcccggccgagcacttcccggcgatggttggccgcatctcgttcttcgtgaacgccggcatccgcttcgtcaccgagatgtgcaagatgcgcgccttcgtggacctctgggacgagatctgccgcgaccgctacggcatcgaggaggagaagtaccgccgcttccgctacggcgtgcaggtcaactcgctgggcctcaccgagcagcagccggagaacaacgtgtaccgcatcctgatcgagatgctcgccgtcaccctgtcgaagaaggcccgcgcccgcgccgtccagctcccggcctggaacgaggccctcggcctcccgcgcccgtgggaccagcagtggtcgctccgcatgcagcagatcctcgcctacgagtcggacctcctcgaatacgaggacctcttcgacggcaacccggccatcgagcgcaaggtcgaggccctgaaggacggcgcccgcgaggagctcgcccatatcgaggcgatgggtggcgccatcgaggccatcgactacatgaaggcccgcctcgtggagtcgaacgccgagcgcatcgcccgcgtggagaccggcgagaccgtggtcgtcggcgtcaaccgctggacctcgggcgccccgtcgccgctcaccaccggcgacggcgccatcatggtcgccgacccggaggccgagcgcgatcagatcgcccgcctcgaagcctggcgcgccggccgcgacggcgccgccgtcgccgccgccctcgccgagctccgccgcgccgccacctcgggcgagaacgtcatgccggcctcgatcgccgccgccaaggccggcgccacgaccggcgagtgggccgccgagctccggcgcgccttcggcgagttccgcggcccgaccggcgtcgcccgcgccccgtcgaaccgcaccgacggcctcgatccgatccgcgaggccgtgcaggccgtctcggcccgcctcggccgcccgctcaagttcgtcgtcggcaagccgggcctggacggccattcgaacggcgccgagcagatcgccgcccgcgcccgggactgcggcatggacatcacctacgacggcatccgcctcaccccggccgagatcgtcgccaaggccgccgatgagcgcgcccatgtgctgggcctctcgatcctgtcgggctcgcacatgccgctggtcaccgaggtcctggccgagatgcgccgcgccggcctcgacatgccgctcatcgtcggcggcatcatcccggaggaggacgccgccgagctgcgcgcctcgggcgtcgccgccgtctacaccccgaaggacttcgagctgaaccgcatcatgatggacatcgtgggcctggtggaccgcaccgccctcgccgccgagtgagatcctgaactagtcctgcaggtaccgtaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctaggagcttgcggccctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcgaggctccgcgccctcgatccgaccgccccccacaaggctggggcggatcgggggcgcacggcctgaagggtgcaggggaaccctgccgcacgcaaactcgcagagtttgcatagtgtgcgccattcagataccttcatggcaggccccaccgtggattttgctgtcatggttcgattcatcacgacgcccgcaacgccgactgcacagcggcccgcaccacgaacgcctgcgcagcaggaagctgcggctgaactgaaggctgcgcgaactgccgaagcagcccgccgcctcgacacccggcgcaaggtggtgctcgggggcgcgctcctggcactggcagaacgtgatgcagcagcagccgcgatgctggcccggctgaagcagatgctgacccgcccagcagatcggaagctgttcgcggatctacccgatggctgacggtgcaatcttccacctcgatgtgcgcacggtcagccggtcagatggccgctcggctgtcgccgctgccgcctatcggtcggccagccgtctgcacgatgcacgcaccggcctgacccacgacttcgcgcgcaagcggggcgtcttgacaaccttcatcgccgccccggacggctgcgactggatcacagacagggacacgctctggaacgcggctgaggcggcagagaagcgcaagaacagcaccgtggcccgtgagtggctggtggccctccctgacgctctggatgctacacagcgggccgacctggcgcgggcgctggcggtcgaactggcgcagcggttcggggtggccgtggatgtggcaatccacgcagcttcgggcgagggcgaccagcggaaccatcacgcgcatttgctgaccacgacgcgcaccgcaggcccggacgggctgggcgacaagacccgcgttctggatgcagccaaaacggggggcgcagaaatctacgccatgcgggcttggtgggcgggcacggtcaacgatgcactcgcggccgcacaaagctcggcccgcgtcgatcaccgccgcatagccgtgatcgccgccgatgcgcgggccgaggccgaggcgctggagaaacaggccaaagccgtcgaaaccctgaacgccaagcccgccgacgccggggggctgtggaagggccttggcagcgccgcccgcgcaatatgggaaggcggcatgaccgccctcaaatcgaccgccgagacagccaacgagctgcgggagaaggccaacgagctgcgacagaaggcagcccgctacacccgcgccaccgaacctcaccacggccccgtcctgacggccttcttgcggcgcatggctcccgtctgggcggagcaggccaaggccgaggaagcccggagagccgcgcaggaggccgcacagcgcgcactgatggccgagagggctgaacaggcccggaaggccaaggaagcctctgaggccgctcgccgggccgctacggcagcgcgagaggccgaggaagcccgcgaacgctgcgcccgcgaggtgttgccgctcatccgccgcgcccggcacgacccgatcaccgccgaagtgatcaagcgggagggcatcgacctcgaccgccccgaccaacaggtggcccgcgacccgatctggctcatggccggacgccaagatcaggcgcacccggtctggctgatcgtccacgcctacaccgccgcgcgcgacaggcacgcgaacctgctggcgagaatcgaagccaaaaccgcagcccagatcgccgctgccgccaaaccaaagcccaagcctgccagtcccgccccgaccgaacccgaacccgatccagacccaccgccccggcgggattggggcgggccgtctggaccctgatccaccccccattctcagcaactccgagccagccagaggcgctggctcggggcatctggacccccttcctcaccccaaattttcacgaaaaaggggcgcagcccttcttgttcaataagttctataagttcaggcgaaaatcgcctttcattttcaatggcttgcgcgcctatcggggagaaatggggccgcaaaagggagaaatggggccgcaaaagggagaaatggggccgatagcggctgttgacagggggatattttgccccctacatttctccccatgacaaagaccacgcaaatcgccgctgatcgagcctatgacgaggccaaaaccgtgctgcccgcagaggtggcgcggggggtctacatgcgcaacgcccccagcctcgccgccctcaagctgatgcacctgctgatcggtaaggcgggcgggcgtatggccgaacccattcggcacgaaatccgcctctcagacatccgcaagatcgacgggatgaagaaccatgacagggccagcctcaccccccttttcgcggaattgtcggcggctgtcctgacccatgatgaccccgaaaaaatggtcgtgaccattggcggcttgctggacttcgcccgcatcgactaccgccacgaagtcagcaacgatctgctggtgtcgtggtattttggcggggccttccgcgagatggccgagaagtcctgccactgggcgatcctcgaccggcagaccgtttttgccctgtccagcaagtattcgatcctgctgttccagcacatcgccagcctcgtgaacctcgatcacgtcgccagcaagaccttcaccgtggccgaactgcgggccgtgctaggggtgccgcacggaaagatggagcggtggaacgacgtgaaccgctttgcgctgacgcccgcgattgccgagatcaaccagacctcgcgcctgatcctgaccgcgaccccgaacaagatcgggcggacggtcgccagcgtgacaatcacgtgggcggagaagcctcccgagggcaagcaggaagccaagcgcgaactggaccgccccaaggtgggccgagaggcgcggcgcaacggctcagccgagaccgtggcgcgggccttcccgagctcgggcagcatcgagttcgatgaccactggaaggccctcaagcgggcggcgggctgcaacatggacaacaccatgattgccgagaagttccgggcgtggtgcgccagcaaggggctttccctcgacggccgaaacatcgagcaggcgttcagcaacttctgcgccacagtgggcaaggtctgaggccgcgcccgctgccagtgacccgccgccgccgaccgtgagcgggccgcagcccttccagcgccgcccaagagggcggcaagccccggtctgccccctcgtcgcccgcaaagcgggcagggggcagaccggggcgctggaacctttgggctgctttgacggccagacagcccccgccgcgcaagcgcgccattcccccacccgtggccgggcgggggaatggtaaagcatgtgggggcagtccagccgtcaatccgcgctccgcaccacggcgcctcgagcaacaccttcttcacgaggcagacctctcgacggagttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagctcgcacattcagcagcgtttttcagcgcgttttcgatcaacgtttcaatgttggtatcaacaccaggtttaactttgaacttatcggcactgacggttactgattttgaacttttgctttgccacggaacggtctgcgttgtcgggaagatgcgtgatctgatccttcaactcagcaaaagttcgccaatacgcaaaccgcctctccccgcgcgttggccgattcattaatgcag

SEQ ID NO: 13 - DNA, 2067 bp, synthetic construct
  source 1..2067: mol_type = other DNA
atgtcggcccaggcctcggtcgccgaggtcaagcgcgataagccgtggatcatccgcacctacgccggccattcgaccgccgccgagtcgaacaagctctaccgcggcaacctggccaagggccagaccggcctctcggtcgccttcgatctcccgacccagaccggctacgatccggatcatgagctggcccgcggcgaggtcggcaaggtcggcgtctcgatcgcccatctcggcgatatgcgcgccctcttcgatcagatcccgctcgcccagatgaacacctcgatgaccatcaacgccaccgccccgtggctcctctcgctctacctcgccgtcgccgaggagcagggcgccccgctcgccgccctccagggcaccacccagaacgatatcatcaaggagtacctgtcgcgcggcacctacgtcttcccgccggccccgtcgctccgcctcaccaaggatgtcatcctgttcaccaccaagaacgtcccgaagtggaacccgatgaacgtctgctcgtaccacctccaggaggccggcgccaccccggtccaggagctctcgtacgccctcgccatcgccatcgccgtcctcgataccgtccgcgatgatccggatttcgacgaggcctcgttctcggatgtgttctcgcgcatctcgttcttcgtcaacgccggcatgcgcttcgtcaccgagatctgcaagatgcgcgccttcgccgagctctgggatgagatcgcccaggagcgctacggcatcaccgatgccaagaagcgcatcttccgctacggcgtccaggtcaactcgctcggcctcaccgagcagcagccggagaacaacgtccatcgcatcctgatcgagatgctcgccgtgaccctctcgaagcgcgcccgcgcccgcgcggtccagctcccggcctggaacgaggccctcggcctcccgcgcccgtgggatcagcagtggtcgatgcgcatgcagcagatcctcgccttcgagaccgacctcctcgaatacgacgacatcttcgacggctcgaccgtcatcgaggcccgcgtcgaggccctcaaggagcagacccgcgccgagctcacccgcatcgccgagatcggcggcgccgtcaccgccgtcgaggccggcgagctcaagcgcgccctcgtcgagtcgaacgcccgccgcatctcggccatcgagaagggcgagcagatcgtcgtcggcgtcaacaagtggcagcagggcgagccgtcgccgctcaccgccggcgatggcgccatcttcaccgtctcggagaccgtcgagatggaggccgagacccgcatccgcgagtggcgctcgaagcgcgatgagcgcgccgtcggccaggccctcgccgatctcgaacaggccgcccgctcgggcgccaacatcatgccgccgtcgatcgccgccgccaaggccggcgtcaccaccggcgagtggggccagcgcctccgcgaggtcttcggcgagtaccgcgccccgaccggcgtcaccctccagaccgtcacctcgggcgccgccgaggatgcccgcctcctcatcgccgatctcggcgagcgcctcggcgagaccccgcgcctcgtcgtcggcaagccgggcctcgatggccattcgaacggcgccgagcagatcgccctccgcgcccgcgatgtcggcttcgatgtcacctacgacggcatccgccagaccccgaccgagatcgtcgccaaggccaaggagcgcggcgcccatgtcatcggcctctcggtgctctcgggctcgcatgtcccgctcgtccgcgaggtcaaggccaagctccgcgaggccggcctcgatcatgtcccggtcgtcgtcggcggcatcatctcgaccgaggatgagctcgtcctgaagaacatgggcgtcaccgccgtgtacaccccgaaggattacgagctcgacaagatcatggtcggcctcgccaaggtggtcgagcgcgccctcgataagcgcgccgccgatcgcgccgataccgaggccggcgtcccgggcgccccgaagcgcaacgagtcgggcgcccaggtcttctga